(19) United States
(12) Patent Application Publication
(10) Pub. No.: US 2018/0171302 A1
(43) Pub. Date: Jun. 21, 2018

(54) Genetically Modified IPS Cells That Carry a Marker to Report Expression of Neurogenin3, TPH2, FOXO1 and/or Insulin Genes

(71) Applicant: Domenico Accili, New York, NY (US)

(72) Inventor: Domenico Accili, New York, NY (US)

(21) Appl. No.: 15/739,443

(22) PCT Filed: Jun. 27, 2016
(86) PCT No.: PCT/US16/39569

§ 371 Date: Dec. 22, 2017

Related U.S. Application Data
(60) Provisional application No. 62/185,555, filed on Jun. 26, 2015.

(51) Int. Cl. C12N 5/074; C12Q 1/6897

(52) U.S. Cl. CPC C12N 5/0696; C12Q 1/6897; C12N 2510/00; C12N 2310/20; C12Q 2600/158

(57) ABSTRACT

Provided herein are insulin-negative cells that have been genetically modified to report expression of one or more target genes. Exemplified are reporter cell lines that provide a readout of Ngn3, Foxo1 or Tph2 expression. Reporter cells are used to screen for agents that affect expression of one or more of these genes to identify agents capable of converting gut progenitor cells to insulin-positive cells.

### CROSS REFERENCE TO RELATED APPLICATIONS

This application claims the benefit of provisional application, 62/185,555 entitled “Genetically Modified IPS Cells That Carry A Fluorescent Marker In The Neurogenin3, Tph2, Foxo1 And Insulin Genes,” filed Jun. 26, 2015, the entire contents of which are incorporated herein.

### STATEMENT OF GOVERNMENTAL INTEREST

This invention was made with Government support under Contract No. DK58282 awarded by the National Institutes of Health. The Government has certain rights in the invention.

### BACKGROUND

Significant progress has been made toward the generation of pancreatic hormone-producing cells from either embryonic or induced pluripotent stem cells (iPSC) (2-4). However, cells thus generated are often polyhormonal, and are compromised by an indifferent response to glucose, unless transplanted into mice, where they acquire undetermined “maturation” factors (2, 3).

A continually renewed source of endocrine progenitors with molecular features similar to pancreatic endocrine progenitors is found in the intestine, the site of the body's largest endocrine system In mice, genetic inactivation of Foxo1 in intestinal endocrine progenitors results in their expansion and in the appearance of beta-like-cells that secrete insulin in response to physiologic and pharmacologic cues. In addition, these beta-like-cells can readily regenerate to alleviate diabetes caused by the b-cell toxin, streptozotocin (1). In contrast, little is known about whether human gut cells can be similarly reprogrammed to produce insulin-secreting beta-like-cells and whether they would be subject to autoimmune attack.

We have reported that knockout of the gene encoding the transcription factor Foxo1 in endocrine progenitor cells results in the appearance of insulin-producing cells in the gut of mice (1). These cells possess features of highly or fully differentiated b-like-cells and they are able to secrete functionally competent insulin in response to a variety of physiologic and pharmacologic secretagogues. We have also shown that, unlike pancreatic beta-cells, these gut-derived insulin-producing cells regenerate rapidly following ablation by the b-cell toxin, streptozotocin (1). The presence of these cells in a structurally organized physical context may contribute to their enhanced functional qualities (6).

The question raised by these exciting findings is whether there are cells present in human gut that can be converted into viable insulin producing cells that may compensate for impaired pancreatic function. Further, there is a need for in vitro cell system that allows for the study of cellular mechanisms involved in how gut ins− cells convert into ins+ cells. If a cell system could be developed, it could in turn be used to screen for possible agents that target gene expression or protein activity of intermediaries involved in the cellular mechanism directing the conversion of gut ins− cells into gut+ cells.

### BRIEF DESCRIPTION OF THE DRAWINGS

The following figures form part of the present specification and are included to further demonstrate certain embodiments of the present invention. The invention may be better understood by reference to one or more of these drawings in combination with the detailed description of specific embodiments presented herein.

FIG. 1 is a picture of a gel demonstrating successful cutting of the guides for Foxo1 and Insulin by Surveyor Assay for the CRISPR method. FOXO1 and insulin CRSPR mutagenesis. Lanes 1-3: 1) Foxo1 Control 293 DNA only. Expected product 505 bp 2) Foxo1 gRNA #1+Ctrl. Expected products are 419 bp and 85 bp. 3) Foxo1 gRNA #10+Ctrl. Expected products are 391 bp and 113 bp. Lanes 4-6: 4) Insulin Control 293 DNA only. Expected product 851 bp. 5) Insulin gRNA #1+Ctrl. Expected products 392/456 bp. 6) Insulin gRNA #10+Ctrl. Expected products 393/455 bp. Lanes 7-9: C control, G control, C+G Control.

FIG. 2 Insulin expression is associated with 5HT inhibition. A-D, IHC of Insulin (green), FOXO1 (red), and 5HT (white). Green arrowheads denote FOXO⁺ cells that underwent conversion to insulin⁺ cells. Note that they do NOT express 5HT (inset in C). Gray arrowheads denote FOXO⁺ cells that express 5HT. Please note that they DID NOT convert into insulin⁺ cells. The white arrowhead denotes the only 5HT^{±}/insulin⁺/FOXO⁺ cells identified in our experiments, also shown in the inset.

FIG. 3 Gut derivation from the Gfp/Cerulean line (Tph2-tracing). Following differentiation of iPS into gut organoids, we induced the formation of Ins+ cells using a dominant-negative (DN) Foxo1 construct. Green: Anti-GFP/Cerulean; Red: Anti-Insulin, Blue: DAPI.

FIG. 4A. Flow cytometry-based isolation of GFP reporter-labeled Tph2 intestinal cells.

FIG. 4B. The P5 population amounts to ˜3% of all sorted cells, consistent with published data on the frequency of 5HT-producing intestinal epithelial cells.

FIG. 4C shows a table represent the percentage of cells with noted expression profile.

FIG. 5A. qPCR analysis of the P5 population isolated by FACS for expression of Foxo1 and Tph2.

FIG. 5B. qPCR analysis of P5 population for expression of Foxo1 and insulin.

FIG. 6, shows histochemical images of primary gut organoids demonstrating that they contain relevant cell types: Mucin (green, top slide), Lysozyme (green, middle and bottom slides).

FIG. 7. Histochemical images of direct Foxo inhibition in primary organoids subjected to Foxo1 dominant-negative construct at a concentration of 1:2000. Appearance of green shows insulin production. Bottom right slide is merger of other slides.

FIG. 8 shows histochemical images of gut organoids using a much lower concentration of Foxo-A mutant (1:10,000) to avoid cell toxicity due to the adenovirus. At this dilution, the virus had almost no effect.

FIG. 9 shows a different cross-section of gut organoids with the lower concentration of FoxoA mutant referred to for FIG. 8.

FIG. 10 shows histochemical dose-response experiments in which lower adenovirus concentrations were used (1:2,000 top and middle slides; 1:5,000 bottom slide), with non-specific effects on cell survival (fragmented nuclei).

FIG. 11, shows a bar graph representing RNA analysis of the converted primary organoids treated with DN256. 2000×, 5000×, and 10000× denote dilution of the virus. Ryo-insulin indicates the qPCR primer used. These data show that DN resulted in induction of Insulin and Neurogenin, as expected.

FIG. 12 shows a diagram of a schematic involving different reporter cell lines.

FIG. 13 shows a diagram of a general CRISPR modification schematic.

FIG. 14 shows a diagram of a general CRISPR modification schematic.

FIG. 15 shows a diagram of a CRISPR modification of the Tph2 gene along with insertion cassette sequence.

FIG. 16 is a diagram of a schematic showing the arrangement of the PAM sequence for CRISPR-based modifications.

### 1. DEFINITIONS

The term “pluripotent cell” as used herein refers to a cell that has the potential to differentiate into any of the three germ layers: endoderm (interior stomach lining, gastrointestinal tract, the lungs, endocrine pancreas), mesoderm (muscle, bone, blood, urogenital), or ectoderm (epidermal tissues and nervous system). Pluripotent stem cells can give rise to any fetal or adult cell type. Induced pluripotent stem cells are a type of pluripotent stem cells.

The term “multipotent cell” as used herein refers to a cell that has potential to give rise to cells from multiple, but a limited number of lineages.

The term “stem cells” as used herein refers to undifferentiated cells that can self-renew for unlimited divisions and differentiate into multiple cell types. Stem cells can be obtained from embryonic, fetal, post-natal, juvenile or adult tissue.

The terms “iPS cells” or “induced pluripotent stem cells” or “inducible pluripotent stem cells” as used herein refer to stem cell(s) that are generated from a non-pluripotent cell, e.g., a multipotent cell (for example, mesenchymal stem cell, adult stem cell, hematopoietic cell), a somatic cell (for example, a differentiated somatic cell, e.g., fibroblast), and that have a higher potency than the non-pluripotent cell. iPS cells may also be capable of differentiation into progenitor cells that can produce progeny that are capable of differentiating into more than one cell type. In one example, iPS cells possess potency for differentiation into endoderm. iPS cells as used herein may refer to cells that are either pluripotent or multipotent. In one specific example, iPSC cells may be generated from fibroblasts such as according to the teachings of US Patent Publication 20110041857, or as further taught herein.

The term “Progenitor cells” or “Prog” in the gut or in the pancreas as used herein refers to cells descended from stem cells that are multipotent, but self-renewal property is limited. N3 Prog differentiate into pancreatic insulin-producing cells during fetal development, but it remains unclear whether there is pancreatic N3 Prog after birth or whether pancreatic N3 Prog can differentiate postnatally into pancreatic hormone-producing cells under normal or disordered conditions. It should be noted here that enteroendocrine (gut) and pancreas N3 prog have different features, even though they are commonly referred to as N3 cells.

The term “Pancreatic N3 Progenitors” and “Panc N3 Prog” as used herein refers to a subset of insulin-negative pancreatic progenitor cells.

The term “N3 Enteroendocrine Progenitors,” “Ngn3+ Prog” and “N3 Prog” as used herein refers to a subset of insulin-negative gut progenitor cells expressing neurogenin 3 that give rise to Ins-negative gut enteroendocrine cells. It has been discovered that N3 Prog in the gut, hereafter “Gut N3 Prog,” have the potential to differentiate into cells that make and secrete insulin (“Gut Ins⁺ Cells”), but this fate is restricted by Foxo1 during development. “Noninsulin-producing gut progenitor cells” or “Ins⁻ Gut Prog” broadly means any gut progenitor cell that is capable of differentiating into an insulin producing gut cell (Gut Ins⁺ cell), including stem cells and N3 Prog.

The terms “Noninsulin-producing Pancreatic progenitor cells” or “Ins⁻ Pancreatic Prog” as used herein refer to any pancreatic progenitor cell that is capable of differentiating into an insulin producing cell (Panc Ins⁺ cell), including stem cells and Ngn3+ Prog.

The term “Enteroendocrine cells” as used herein refers to specialized endocrine cells of the gastrointestinal tract, most of which are daughters of N3 Prog cells that no longer produce Neurogenin 3. Enteroendocrine cells are Insulin-negative cells (Gut Ins⁻); they produce various other hormones such as gastrin, ghrelin, neuropeptide Y, peptide YY₃₋₃₆ (PYY₃₋₃₆) serotonin, secretin, somatostatin, motilin, cholecystokinin, gastric inhibitory peptide, neurotensin, vasoactive intestinal peptide, glucose-dependent insulinotropic polypeptide (GIP) and glucagon-like peptide-1.

The terms “Gut Ins⁺ Cells” and “Insulin positive gut cells” as used herein refer to any enteroendocrine cells that make and secrete insulin descended from Ins⁻ Gut. The Gut Ins⁺ cells have the insulin-positive phenotype (Ins⁺ ) so that they express markers of mature beta-cells, and secrete insulin and C-peptide in response to glucose and sulfonylureas. Gut Ins⁺ Cells arise primarily from N3 Prog cells. These cells were unexpectedly discovered in NKO (Foxo1 knock out) mice. Unlike pancreatic beta-cells, gut Ins⁺ cells regenerate following ablation by the beta-cell toxin, streptozotocin, reversing hyperglycemia in mice.

The term “LGR5” or “leucine-rich repeat-containing G-protein coupled receptor 5” as used herein means a protein that in humans is encoded by the LGRS gene, and is a biomarker of adult stem cells.

The terms “CRISPR” or “CRSPR” are used interchangeably herein as an abbreviation for Clustered Regularly Interspaced Short Palendromic Repeat, a region in bacterial genomes used in pathogen defense.

The term “Cas” as used herein refers to an abbreviation for CRISPR Associated Protein; the Cas9 nuclease is the active enzyme for the Type II CRISPR system.

The term “CRISPRi” as used herein refers to an abbreviation for CRISPR Interference, using a dCas9+ gRNA to repress/decrease transcription of a gene by blocking RNA Pol II binding.

The term “crRNA” as used herein refers to an abbreviation for the endogenous bacterial RNA that confers target specificity, requires tracrRNA to bind to Cas9.

The term “Cut” in the context of CRSPR/CRISPR as used herein refers to a double strand break, the wild type function of Cas9.

The term “DSB” as used herein refers to an abbreviation for Double Strand Break, a break in both strands of DNA, Cut, 2 proximal, opposite strand nicks can be treated like a DSB.

The terms “Dual Nick(ase)/Double Nick/Double Nicking” as used herein refer to a method to decrease off-target effects by using a single Cas9 nickase and 2 different gRNAs, which bind in close proximity on opposite strands of the DNA, to create a DSB.

The term “gRNA” as used herein refers to a guide RNA, a fusion of the crRNA and tracrRNA, provides both targeting specificity and scaffolding/binding ability for Cas9 nuclease; it does not exist in nature.

The term “gRNA sequence” as used herein refers to the 20 nucleotides that precede the PAM sequence in the targeted genomic DNA. It is what gets put into a gRNA expression plasmid and it does NOT include the PAM sequence.

The term “HDR” as used herein refers to Homology Directed Repair, a DNA repair mechanism that uses a template to repair nicks or DSBs.

The term “InDel” as used herein refers to Insertion/Deletion, a type of mutation that can result in the disruption of a gene by shifting the ORF and/or creating premature stop codons.

The term “NHEJ” as used herein refers to Non-Homologous End-Joining, which is a DNA repair mechanism that often introduces InDels.

The term “Nick” as used herein refers to a break in only one strand of a double stranded DNA that is normally repaired by HDR.

The term “Nickase” as used herein refers to Cas9 that has one of the two nuclease domains inactivated. Examples include RuvC or HNH domain.

The term “Off-target effects” as used herein refers to gRNA binding to target sequences that does not match exactly, causing Cas9 to function in an unintended location. It can be minimized by double-nick.

The term “ORF” as used herein refers to Open Reading Frame, the codons that make up a gene.

The term “PAM” as used herein refers to Protospacer Adjacent Motif, which is a required sequence that must immediately follow the gRNA recognition sequence but is NOT in the gRNA.

The term “RGEN” as used herein refers to RNA Guided EndoNuclease, which is the use of Cas9 and a gRNA, CRISPR technology.

The term “sgRNA” as used herein refers to single guide RNA, the same as a gRNA, which is a single stranded RNA.

The terms “Fluorescent Reporter Gene” and “Reporter Gene” are used interchangeably herein to refer to the fluorescent marker to be inserted into the genome and fused to the target gene to be a readout of target gene expression. In the diagram below it is referred to as a “specific change.”

The term “Specific change,” as used herein refers to any change introduced into the genome. For example the introduction of a reporter gene.

The term “Target locus” as used herein refers to the locus in the genome where the target gene is found.

The term Expression Cassette” as used herein refers to the nucleotide cassette (in embodiments of the invention it is carried by the “repair template”) for incorporation into the genome at the Cas-9 DB cut site (hereafter “cut site”). It contains the reporter gene that is flanked by two homology arms to position insertion of the specific change (i.e. addition of the reporter gene) into the genome.

The term “Repair template” as used herein refers to the gRNA plus the Cas-9 gene and the expression cassette with the DNA template including the reporter gene to be inserted into the genome at the target locus.

The term DNA template as used herein refers to the sequence in the expression cassette comprising the two homology arms plus the specific change to be inserted into the genome at the target locusi.e. the reporter gene sequence in embodiments of the invention.

The term “Target sequence” as used herein refers to the 20 nucleotides in the genome near the cut site that are incorporated into the gRNA to direct the location of incorporation of the repair template (with the expression cassette carrying the reporter gene) to the cut site. The target sequence is in the genomic DNA and is typically part of the gene encoding the “target gene” (Ngn, foxo1, Tph1 and 2 and insulin).

The term “tracrRNA” as used herein refers to the endogenous bacterial RNA that links the crRNA to the Cas9 nuclease; it can bind any crRNA.

### 2. Detailed Description of the Embodiments

Gut endocrine cells are comprised of over twenty distinct and overlapping cell types, originating from Neurogenin3-expressing progenitor cells. As indicated above, we have demonstrated that, among the many different endocrine cell types, there is a single cell type that can be converted into an insulin-producing cell, the serotonin-producing cell. In human gut and gut organoids, FOXO1 expression is restricted to endocrine progenitor and serotonin (5HT)-producing cells. FOXO1 inhibition by a dominant-negative mutant or shRNA-mediated knockdown in these cells results in their conversion into β-like-cells that express all tested markers of mature pancreatic β-cells, produce insulin, and release it in response to secretagogues. Moreover, the conversion process is associated with decreased 5HT content.

It is useful to be able to monitor in real time the conversion of uncommitted insulin-negative gut progenitors “Gut N3 Prog” into insulin-producing cells “Gut Ins⁺ Cells” by monitoring the expression of four critical “target” genes in this process: Neurogenin3 (a marker of endocrine progenitor cells), Thp2 (the rate-limiting enzyme for the production of serotonin), Foxo1 (the driver of the conversion of insulin⁻ gut progenitors to insulin+ gut cells, and Insulin (the target of this process). This can be accomplished by fusing each target gene to a uniquely detectable fluorescent reporter gene marker that is quantitated as a visual and quantifiable readout of the activity of each modified gene. The fluorescent reporter gene may be inserted via a Clustered Regularly Short Palindromic Repeats (CRISPR), Zinc-finger nuclease or Talen process. Genetically modified human inducible pluripotent cell (iPS) lines were made using CRISPR as is described in detail in Examples 1 and 2 to introduce (knock-in) specific fluorescent reporter genes into the following genes: Neurogenin 3, Foxo1, Tph1 or Tph2, and insulin. Individual reporter cell lines with reporter genes inserted for each of these genes has been generated. It is noted that ifall or a combination of the genes are modified in the cell, then different fluorescent markers that fluoresce at distinct wavelengths are used for each target gene. Gene manipulation is not expected to result in gene dosage effects but, should they occur, it can be detected and CRISPR targeting strategy can be modified using routine experimentation to preserve the integrity of the endogenous allele.

Certain embodiments of the invention are directed to non-insulin-producing cells (insulin-negative/ins⁻ cells) wherein a genomic target gene selected from the group consisting of Neurogenin 3, Thp1, Tph2, Foxo1, and insulin, or combination thereof, has been genetically modified by fusion to a reporter gene (e.g. fluorescent reporter gene) such that expression of the reporter gene is a readout of expression of the target gene. In some embodiments the mRNA encoding the fused gene is in a single reading frame or it is in two reading frames. In some embodiments two or more genomic target genes are genetically modified, each with a different reporter gene. The genetically-modified cell can be a stem cell or progenitor cell, a Neurogenin 3 positive cell, a foxo1 positive cell, a Tph1 positive cell or a Tph2 positive cell. In more specific embodiments, the cell is a gut cell or pancreatic cell. In an even more specific embodiment, the reporter gene is placed immediately upstream (within 10 bp) of a protospacer adjacent motif sequence in the target gene. The reporter gene may be placed immediately adjacent to the 5′ end of PAM sequence.

Certain embodiments are directed to the modified cell in which the fluorescent reporter gene is introduced into the cells by homologous recombination at a double stranded DNA break, for example where the genetic modification is made using a Clustered Regularly Interspaced Short Palindromic Repeats (CRISPR)-associated protein method that implements a Cas protein, such as Cas9.

In an embodiment the CRISPR-associated method comprises introducing into the cell: (i) a first expression construct comprising a first promoter operably linked to a first nucleic acid sequence encoding a CRISPR-associated (Cas) protein, and (ii) a second expression construct comprising a second promoter operably linked to a second nucleic acid sequence encoding a genomic RNA (gRNA) sequence complementary to a first particular genomic target sequence. In an embodiment, the genomic target sequence in the modified cells is immediately flanked on the 3′ end by a Protospacer Adjacent Motif (PAM) sequence in the genome which is needed for Cas production of the double stranded cut. The gRNA used to modify the cells comprises a nucleic acid sequence encoding a Clustered Regularly Interspaced Short Palindromic Repeats (CRISPR) RNA (crRNA) and a trans-activating CRISPR RNA (tracrRNA). In a more specific embodiment, the CRISPR method further comprises (iii) introducing into the cell a large targeting vector (LTVEC), comprising a first gene encoding a first fluorescent reporter targeted to a first target gene that is immediately flanked on the 3′ end by a Protospacer Adjacent Motif (PAM) sequence, selected from the group consisting of Neurogenin 3, Tph1 or Tph2, Foxo1 and insulin.

In a more specific embodiment, Tph2 is the target gene to monitor serotonin-producing cells because it is the isoform that is upregulated by FOXO1 inhibition, thereby generating increased levels of endogenous serotonin. It is believed to be the most sensitive indicator of successful FOXO1 inhibition-dependent conversion. Alternately, TPH1 has been implicated in 5HT generation in the intestine (20). However, both TPH1 and TPH2 are expressed in β-cells (8) and in certain gut enteroendorine cells and either or both can be targeted with the CRISPR method.

Any fluorescent reporter gene is suitable for fusion in embodiments of the invention including, but not limited to, cyan fluorescent protein, far red fluorescent proteins, green fluorescent proteins, orange fluorescent protein, yellow fluorescent protein, cerulean fluorescent protein, photoswitchable fluorescent protein, red fluorescent protein, pamcherry (a photoactivatable fluorescent protein (pafp) derived from the red fluorescent protein mcherry.

In an embodiment, the iPS cells are genetically modified using homologous recombination at a double-stranded DNA break, that are preferably made using the CRISPR method or the Clustered Regularly Interspaced Short Palindromic Repeats (CRISPR)-associated (Cas) protein method, TALEN method, Zinc-finger nuclease method, or any other method that is known in the art. In an embodiment, the Cas protein is Cas9 (more details are presented below).

In certain examples, the reporter gene was introduced into the genome for each target gene in exon 1. This places the reporter between the promoter and endogenous target gene in the genome, or at the end of the target gene before the stop codon. In either way, the reporter gene fused to the endogenous target gene provides a readout of target gene expression and is driven by the endogenous target gene promoter.

In an embodiment, the fluorescent reporter gene is introduced into the progenitor cells in an expression construct (also called a cassette) in the repair template. It is not necessary to include a promoter if the reporter gene is inserted under the expression of the endogenous target gene promoter as described. In another embodiment, the progenitors are modified to express two or more target genes each of which has been fused to a different fluorescent reporter gene. In a further embodiment the progenitor cells are modified to express three or all four target genes fused to respective unique fluorescent reporter genes.

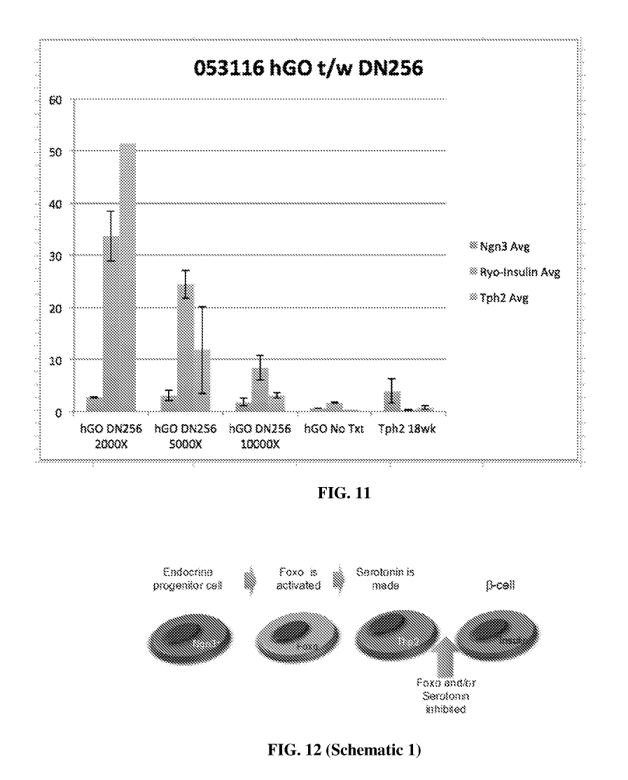

As the schematic in Schematic 1 in FIG. 12 shows, the strategy of screening methods (described below) is to discover drugs that turn non-insulin-producing iPS into cells that even eventually make insulin (such as insulin+ gut enteroendocrine cells (with β-cell properties)). Genetically modified human iPS cells permit transitions through different differentiation stages using different fluorescent reporter genes fused to the four target genes. Ngn3+ progenitor cells are labeled for example with GFP can be isolated using FACS based on GFP expression, and then cultured to grow them in large numbers as a Ngn3+-enriched population confirmed. As Ngn3+ progenitors (green) differentiate, they will turn on Foxo1 (orange) then they will express Thp2 (serotonin, cerulean), and when Foxo1 is turned off, they will finally make insulin. The timing of the appearance of FOXO1 and TPH2 may or may not be sequential, but it is expected that both will be present in the same cell at the same time. Lastly, insulin will appear, and this may or may not be associated with loss of FOXO1 or TPH2, but loss is expected.

### A. Screening Assay and Methods

Based on the fluorescent markers utilized in Schematic 1, as cells differentiate, they will first turn green (Ngn3+-GFP), then yellow (Foxo1+-orange) plus (Tph2+-cerulean), and finally red (insulin+ Red Fluorescent protein). For purposes of describing Schematic 1, the cells would be assumed to contain all four of the fluorescent markers. When insulin reporter cells fluoresce in the range of the insulin gene reporter, the yellow fluorescence engendered by the activity of Foxo reporter cells or serotonin production will disappear because these two genes are not expressed (or are expressed at very low levels in insulin+ gut cells.) Screens can be set up to identify compounds that induce expression or inhibition of any one or more of the four target genes either individually (e.g. using separate reporter cell lines) or sequentially. For example, similar to what has been done to generate insulin-producing cells from embryonic stem cells, a protocol can be used in which cells are first treated with Notch inhibitors to drive their differentiation into Ngn3+ cells, then with inhibitors of Wnt signaling to induce Tph2 expression, then with inhibitors of 5HT synthesis, signaling, or activators of 5HT degradation to induce pancreas-specific endocrine lineages. Another embodiment is directed to a screening assay using isolated, genetically modified iPS cells grown in a monolayer to detect compounds that affect their conversion into specific cell types (Neurogenin3+, Tph1 or 2+, Foxo1+, Insulin+), or that cause the inhibition of expression of a target gene. In addition to allowing for the testing of Foxo1 inhibitors for cell-conversion purposes, these cell lines would enable the testing of any agent or method-independent of Foxo1—that affects the conversion of one cell type to another, including the differentiation of these cells into any gut endocrine cell type, which in turn could be useful to develop new anti-diabetic therapies.

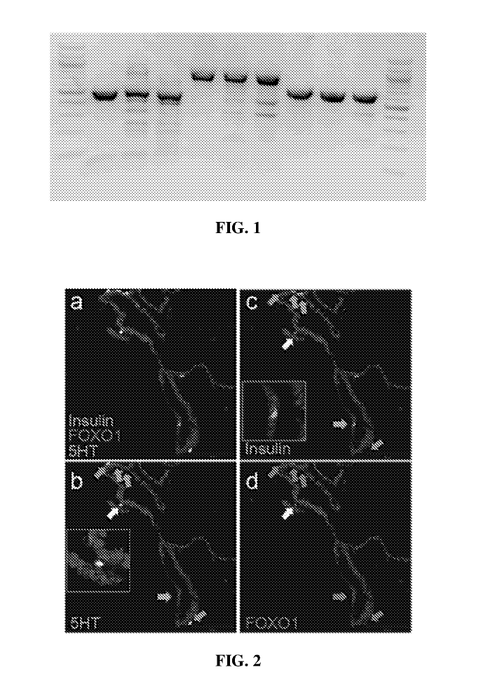

The expected outcome in cells bearing CRISPR-modified alleles of both NEUROG3 and insulin, are the appearance of doubly fluorescent cells after FOXO1 inhibition only if this cell type is the target of FOXO inhibition-dependent generation of β-like-cells. In other words, if NEUROG3 is active at the time of conversion into insulin⁺ cells, this means that trans-differentiation is occurring in endocrine progenitors. In cells bearing CRISPR-modified alleles of TPH andinsulin, it can be determined whether acquisition of insulin immunoreactivity precedes or follows acquisition of 5HT immunoreactivity, and whether upon the activation of insulin, 5HT levels (determined for example by immunohistochemistry) decrease, as in FIG. 2a-d. Based on the data, it is expected that 5HT levels decrease prior to insulin production.

It is expected that the some of the active agents identified in screening assays are subsets of overlapping hits (compounds that generate insulin by inhibiting Foxo1 and/or serotonin as well as a subsets of compounds that gives rise to insulin-producing cells without inhibiting Foxo1 or serotonin).

In specific embodiments, the reporter cell lines described herein can then be grown as gut organoids or monolayers of phenotypically identical cells for further screening studies. In certain embodiments, a method is provided that utilizes the iPS cells and genetic modifications schemes described herein to generate culture systems in which clonal endocrine cells can be isolated (by virtue of having the fluorescent marker) and grown as a monolayer, gut organoid or other culture. These cells may be used in assays to detect compounds that affect their conversion into specific cell types (Neurogenin3, Tph1, Foxo1, Insulin). In addition to allowing for the testing of Foxo1 inhibitors for conversion purposes, these cell lines enable the testing of any method—independent of Foxo1—to effect the conversion. Further, the cell lines enable the testing for compounds that promote the differentiation of these cells into any gut endocrine cell type, which in turn would provide for the development of new anti-diabetic therapies.

Accordingly, in one embodiment, provided is a method for identifying an agent that modulates expression in a cell of at least one genetically modified genomic target gene selected from the group consisting of Neurogenin 3, TPH2, TPH1, FOXO1, and insulin. The target gene is fused to a reporter gene (e.g. fluorescent reporter gene) such that expression of the reporter gene corresponds to expression of the target gene so as to indicate expression of the target gene. In a more specific embodiment, the method involves (i) culturing the cell under conditions that permit target gene expression indicated by detectable fluorescence from the reporter gene, (ii) contacting the cell with a test agent in an amount and for a duration of time that permits the test agent to modulate target gene expression in the cell, and (iii) selecting the test agent if it modulates target gene expression, indicated by a change of in the amount of the fluorescence in the cell. Either a reduction or increase in gene expression as a result of the test agent can be detected. In an even more specific embodiment, the cell involves a plurality of cells. Further, the plurality of cells may be disposed on a substrate, such as a monolayer culture in a dish or similar container, or in the form of a gut organoid. In an even more specific embodiment, the target gene is TPH2.

Another embodiment pertains to an insulin-negative gut cell genetically modified to comprise a reporter gene fused to a TPH2 gene or insulin gene such that expression of the reporter gene occurs with expression of TPH2 or insulin.

### B. CRSPR/CRISPER Technology

CRISPR is an RNA-guided gene-editing platform that makes use of a bacterially derived protein (Cas9) and a synthetic guide RNA to introduce a double strand break at a specific location within the genome. Editing is achieved by transfecting a cell with the Cas9 protein along with a specially designed guide RNA (gRNA) (in a repair template) that directs the double-stranded cut through hybridization with its matching genomic sequence in the target genome at the target locus. https://www.addgene.org/CRISPR/guide/ was used in some of the following description of CRISPR.

There are two distinct components to this system: (1) a guide RNA and (2) an endonuclease, in this case the CRISPR associated (Cas) nuclease, Cas9. The guide RNA is a combination of the endogenous bacterial crRNA and tracrRNA into a single chimeric guide RNA (gRNA) transcript. The gRNA combines the targeting specificity of the crRNA with the scaffolding properties of the tracrRNA into a single transcript. When the gRNA and the Cas9 are expressed in the cell, the genome is modified such as by knocking in a reporter gene to be fused to a target gene at the cut site. A Target sequence can either be modified or disrupted if desired. In embodiments of the invention a reporter gene is introduced into the genome at the target sequence without disrupting the endogenous target gene that either precedes or follows the target gene. The Cas9 nuclease activity (cut) is performed by 2 separate domains, RuvC and HNH. Each domain cuts one strand of DNA and each can be inactivated by a single point mutation.

A typical embodiment involving CRSPR mutagenesis would involve the following basic steps:

1) Choose a desired region of mutagenesis in the target gene (this means the placing of the double stranded cut). In embodiments of the invention, this is either (i) at the end of the target gene (such as Ngn3+) before the stop codon where the fluorescent reporter gene will be inserted and fused to the target gene so that it is transcribed together with the target gene, to serve as a readout of target gene expression and enable visual monitoring of target gene expression (ii) in exon 1 of the target gene which will put the reporter gene between the endogenous promoter and the target gene again permitting fusion and tandem transcription, or (iii) after an IRES (Internal ribosome entry site) to generate a bi-cistronic mRNA that encodes both the endogenous (i.e. Ngn3+ protein) and the fluorescent protein as separate proteins where the mRNA reads off of multiple starting points.

2) Copy a 20 nucleotide genomic “target sequence” in the desired region of mutagenesis, which site needs to be followed by a PAM to direct the Cas9 to the desired location of the cut site. For successful binding of Cas9, the endogenous genomic target sequence must also be immediately followed by the correct Protospacer Adjacent Motif (PAM) sequence (see more description below of PAM).

3) Paste the target sequence into a gRNA-generating algorithm (such as described at crispr.mit.edu)

4) gRNA will bind upstream of PAM (NGG)

5) Choose optimal guide (rated by predicted off-target effects). Thus the gRNA/Cas9 complex is recruited to the target sequence at the target locus by the base-pairing between the gRNA sequence and its complement in the target sequence in the genomic DNA.

The binding of the gRNA/Cas9 complex localizes the Cas9 to the genomic target sequence so that the wild-type Cas9 can cut both strands of DNA causing a Double Strand Break (DSB). Cas9 will cut 3-4 nucleotides upstream of the PAM sequence. A DSB (double stranded break) can be repaired through one of two general repair pathways: (1) the Non-Homologous End Joining (NHEJ) DNA repair pathway or (2) the Homology Directed Repair (HDR) pathway. The NHEJ repair pathway often results in inserts/deletions (InDels) at the DSB site that can lead to frameshifts and/or premature stop codons, effectively disrupting the open reading frame (ORF) of the targeted gene). The HDR pathway requires the presence of a “repair template” that carries the expression cassette with the DNA template for the reporter gene to be inserted and two homology arms to position insertion of the reporter gene into the genome at the cut site. The repair template targets the reporter gene to the site of insertion and fixes the DSB made by Cas-9. HDR faithfully copies the reporter gene sequence to the site of insertion at the target sequence. This method is used in embodiments of the present invention. Note that there are libraries of tens of thousands of guide RNAs that are now available.

The expression cassette that carries the DNA template for the gene encoding the fluorescent reporter gene and the two homology arms, is normally included in the repair template that carries gRNA/Cas9. The homology arms have a high degree of homology to a region in the endogenous target gene to faithfully direct the insertion of the specific nucleotide changes (introduction of the reporter gene) to the cut site. The length and binding position of each homology arm is dependent on the size of the change being introduced. The desired modification in the genomic DNA is then confirmed experimentally.

The cut site can be located so that the reporter gene is introduced into the target gene downstream from the endogenous gene promoter, so that the expression cassette does not need a promoter. It can also be inserted upstream from the stop codon for the endogenous target gene at the end of the gene. Fusion of the reporter gene to the target gene will enable transcription of the reporter together with the target gene so that the endogenous gene and reporter gene are transcribed as a single protein and the reporter is a readout of target gene expression.

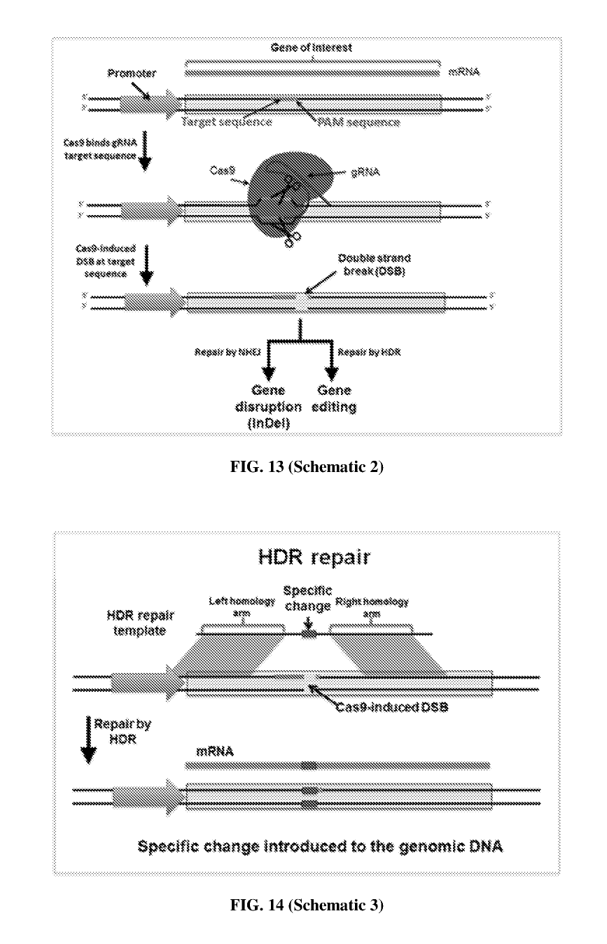

In the schematic 2 and 3 shown in FIGS. 13 and 14, respectively, (used only as a basic illustration of the CRISPR method), the “specific change” is analogous to the DNA template gene encoding the reporter gene in this application. Schematic 2 shows insertion of the specific change into the middle of the target gene. As previously described, in embodiments of the invention the repair template is not inserted into the middle of the target gene as this would cause disruption of the target gene which is not desired.

In an embodiment, the expression cassette carrying DNA template for the reporter gene sequence (in the repair template) may optionally have a PAM site that has been modified so that it is not susceptible to Cas9 cleavage. This enables one to go back and modify the endogenous gene/reporter gene/or gene combination at a later time.

When designing a repair template for genome editing by HDR, it is important that the repair template (carrying the reporter gene to be inserted) either does not contain an unmodifiedd PAM sequence because this would cause the template itself to be cut by the Cas9. Instead if it is desired to include a PAM in the DNA template, it should be sufficiently modified to ensure it is not cut by Cas9. For making mutations in PAM in the repair template (which is optional) is to mutate the PAM ‘NGG’ sequence in the HR template for example by changing it to ‘NGT’ or ‘NGC’ to protect the HR template from the Cas9. If PAM is within coding region the mutation should be a silent mutation.

In embodiments of the present are invention each of the homology arms in the DNA template typically have about 0.5-1 kb of genomic sequence and are homologous, preferably exactly homologous, a portion of the endogenous genomic sequence. This region of homology is crucial for the success of the homologous recombination reaction, as it serves as the guide template for specifically targeting the DNA template in the expression cassette to the site of insertion into the genonme. The actual regions of recombination at the 5′ and 3′ of the target site can vary widely. Some use homology arms that are less than 15 bp away from the double strand break site. Longer distances can be used in embodiments of the present invention for introducing a selection marker gene, but ideally the homology arms should be no more than 100 bp away from the DSB.

The CRISPR method provides a seamless, in-frame junction between the target endogenous coding sequence (Ngn, Foxo1, Tph1 or 2, Insulin) fused to the fluorescent reporter, such as the GFP marker.

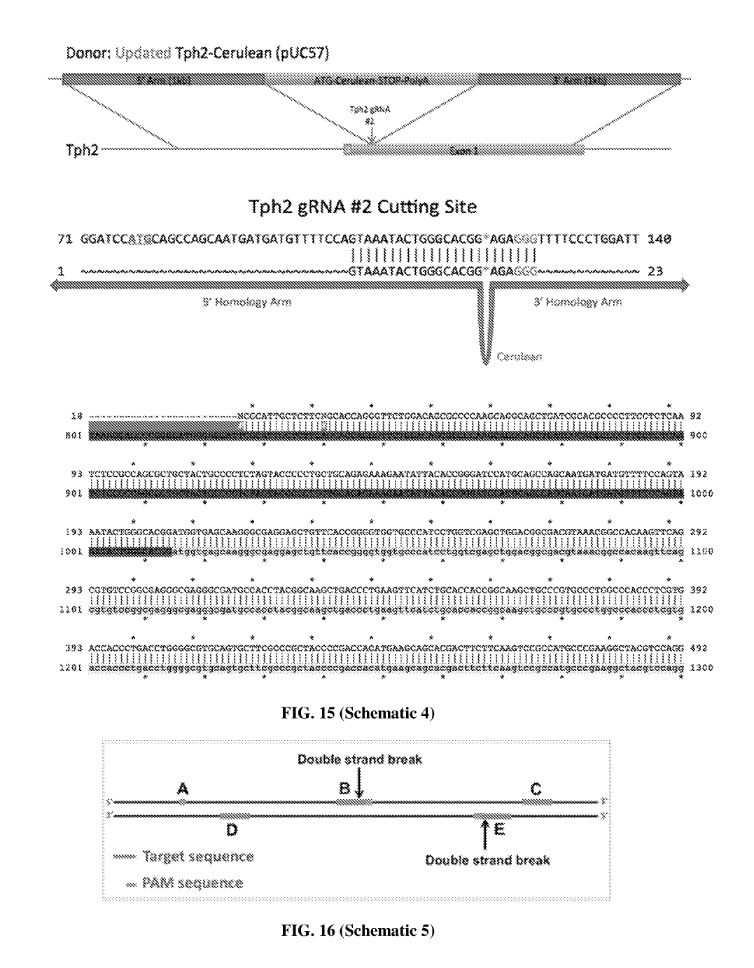

The CRISPR mutagenesis experiments reported herein to introduce the various reporters used the gRNAs as listed in Example 2 below. Schematic 4 shown in FIG. 15 is a drawing showing part of the repair template carrying the DNA template encoding the cerulean reporter gene and the 5′ and 3′ homology arms for insertion into genome at exon 1 of the Tph2 endogenous target gene. The homology arm is shown in dark blue and the cerulean sequence is shown in cyan.

Software for Designing gRNAs

Various Software programs are available for designing gRNAs for a given gene.

Feng Zhang lab's Target Finder Identifies gRNA target sequences from an input sequence and checks for off-target binding. Currently supports: Drosophila, Arabidopsis, zebrafish, C. elegans, mouse, human, rat, rabbit, pig, possum, chicken, dog, mosquito, and stickleback.

Michael Boutros lab's Target Finder (E-CRISP) Identifies gRNA target sequences from an input sequence and checks for off-target binding. Currently supports: Drosophila, Arabidopsis, zebrafish, C. elegans, mouse, human, rat, yeast, frog, Brachypodium distachyon, Oryza sativa, Oryzias latipes.

RGEN Tools: Cas-OFFinder Identifies gRNA target sequences from an input sequence and checks for off-target binding. Currently supports: Drosophila, Arabidopsis, zebrafish, C. elegans, mouse, human, rat, cow, dog, pig, Thale cress, rice (Oryza sativa), tomato, corn, monkey (macaca mulatta).

CasFinder: Flexible algorithm for identifying specific Cas9 targets in genomes Identifies gRNA target sequences from an input sequence, checks for off-target binding and can work for S. pyogenes, S. thermophilus or N. meningitidis Cas9 PAMs. Currently supports: mouse and human

CRISPR Optimal Target Finder entifies gRNA target sequences from an input sequence and checks for off-target binding. Currently supports over 20 model and non-model invertebrate species.

The Protospacer Adjacent Motif (PAM) Sequence

For Cas9 to successfully bind to DNA, the target sequence in the genomic DNA must be complementary to the gRNA sequence and must the target sequence must be immediately followed by the correct protospacer adjacent motif (PAM sequence). The PAM sequence is present in the DNA target sequence but not in the gRNA sequence. Any DNA sequence with the correct target sequence followed by the PAM sequence will be bound by Cas9.

As shown in schematic 5 in FIG. 16, the target sequence is followed by the PAM sequence at two separate locations (B and E). Cas9 will ONLY cut at B and E. The presence of the target sequence without the PAM following it (C and D) is NOT sufficient for Cas9 to cut. The presence of the PAM sequence alone (A) is not sufficient for Cas9 to cut.

The PAM sequence varies by the species of the bacteria from which the Cas9 was derived. The most widely used Type II CRISPR system is derived from S. pyogenes and the PAM sequence is NGG located on the immediate 3′ end of the gRNA recognition sequence. The PAM sequences of other Type II CRISPR systems from different bacterial species are listed in the Table 1 below. It is important to note that the components (gRNA, Cas9) derived from different bacteria will not function together. Example: S. pyogenes (SP) derived gRNA will not function with a N. meningitidis (NM) derived Cas9.

The majority of the CRISPR plasmids in Addgene's collection are from S. pyogenes unless otherwise noted.

CRISPR Delivery Options

Once a target site has been identified, it's important to consider delivery options. Generally, CRISPR constructs can either be transfected into cells for transient expression or infected with virus. If using a retrovirus or lentivirus, it is not advisable to use the resulting cells for long-term (months, years) studies, due to the potential effects of constitutive Cas9 expression and resulting accumulation of off-target effects. Transient expression options, then, such as transfection, electroporation, or non-integrating viruses such as AAV or Adenovirus, are the most appropriate choices for creation of a stable cell line with an engineered change. The repair template for homologous recombination can be either a plasmid or single-stranded oligo co-transfected with the Cas9 and sgRNA. The rate of homologous recombination in a particular cell can be low even with the use of CRISPR technology (<1-5%), and thus cells need to be clonally isolated and screened for successful integration. This step is likely the most time consuming part of this process.

Once a target site has been identified, it's important to consider delivery options. Generally, CRISPR/CRISPER constructs can either be transfected into cells for transient expression or infected with virus. If using a retrovirus or lentivirus, it is not advisable to use the resulting cells for long-term (months, years) studies, due to the potential effects of constitutive Cas9 expression and resulting accumulation of off-target effects. Transient expression options, then, such as transfection, electroporation, or non-integrating viruses such as AAV or Adenovirus, are the most appropriate choices for creation of a stable cell line with an engineered change. The repair template for homologous recombination can be either a plasmid or single-stranded oligo co-transfected with the Cas9 and sgRNA. The rate of HR in a particular cell can be low even with the use of CRISPR technology (<1-5%), and thus cells need to be clonally isolated and screened for successful integration. This step is likely the most time consuming part of this process.

Protocols

Off-Target Effects and Cas9 Nickase

The CRISPR technology is becoming widely-used because of its ease of use and efficacy. However, off-target effects of the Cas9 nuclease activity is a current concern with the use of the CRISPR system. Apparent flexibility in the base-pairing interactions between the gRNA sequence and the genomic DNA target sequence allows imperfect matches to the target sequence to be cut by Cas9. Single mismatches at the 5′ end of the gRNA (furthest from the PAM site) can be permissive for off-target cleavage by Cas9.

Avoiding off-target effects of Cas9 cutting is an important step in designing sgRNAs. While the rules governing off-target effects are still in their infancy, some guidelines have been developed and incorporated into current design algorithms Bioinformatic tools to help identify genomic loci that exhibit the greatest amount of sequence uniqueness include:
Feng Zhang lab: crispr.mit.edu/
Michael Boutros lab: www.e-crisp.org/E-CRISP/designcrispr.html

One method to decrease off-target effects with CRISPR technology is the use of two sgRNAs in combination with a mutated “nickase” version of Cas9. This approach has the benefit of increased specificity and thus a reduced rate of off-target dsDNA breaks. One downside of this approach, though, is that the requirement for two target sites will mean some specific locations are not suitable for creating a dsDNA break. When possible, though, this is the preferred approach for gene editing. Such methods are known in the art.

Cas9 (CRISPR associated protein9) is an RNA-guided DNA endonuclease enzyme associated with the CRISPR (Clustered Regularly Interspersed Palindromic Repeats) adaptive immunity system in Streptococcus pyogenes, among other bacteria. S. pyogenes utilizes Cas9 to memorize and later interrogate and cleave foreign DNA, such as invading bacteriophage DNA or plasmid DNA. Cas9 performs this interrogation by unwinding foreign DNA and checking for if it is complementary to the 20 base pair spacer region of the guide RNA. If the DNA substrate is complementary to the guide RNA, Cas9 cleaves the invading DNA. CRISPR was first shown to work as a genome engineering/editing tool in human cell culture by 2012 by reprogramming a CRISPR/Cas system to achieve RNA-guided genome engineering. Jinek M, et al., (August 2012). “A programmable dual-RNA-guided DNA endonuclease in adaptive bacterial immunity”.Science 337 (6096): 816-821.

### 3. Detailed Description of the Experimental Results

### A. Summary of Experimental Results

1. Human induced pluripotent stem cells (iPSCs) were generated from donor tissue from healthy patients.

2. iPSCs were genetically modified using CRISPR techniques to produce reporter cell lines with fluorescent markers placed so as to generate an expression readout of the Ngn3, Foxo1, or Tph2 genes.

3. Gut organoids were successfully produced from the human iPSCs.

4. Insulin-producing cells were successfully produced in gut organoids generated from CRSPR-modified cells via Foxo1 ablation. Tph2 reporter cell line was differentiated into gut organoids, then the gut organoids were subjected to dominant-negative (DN) Foxo1 mutant to induce the formation of insulin-positive cells. Tph2 expression decreased as insulin production increased supporting the hypothesis that the 5HT pathway is suppressed as gut cells convert to insulin producing cells.

5. Histochemical analysis of primary gut organoids subjected to a DN Foxo1 mutant showed that the Tph2 expression of the cells decreased while the production of insulin increased.

### B. Examples

### Example 1

### Production of IPS Cells for Genetic Modification Studies

Human induced pluripotent stem cells (iPS cells or iPSCs) were generated from fibroblast of three healthy control subjects as previously described (Hua, H., et al. iPSC-derived beta cells model diabetes due to glucokinase deficiency (Hua, H., et al. iPSC-derived beta cells model diabetes due to glucokinase deficiency, J Clin Invest 123, 3146-3153 (2013); Maehr, R., et al. Generation of pluripotent stem cells from patients with type 1 diabetes. Proc Natl Acad Sci U S A 106, 15768-15773 (2009)). Briefly, upper arm skin biopsies were obtained from healthy subjects using local anesthesia. The biopsies were processed as described and placed in culture medium containing DMEM, fetal bovine serum, GlutMAX, and Penicillin/Streptomycin (all from Invitrogen) for 4 weeks3. The CytoTune-iPS Sendai Reprogramming Kit (Invitrogen) was used to convert primary fibroblasts into pluripotent stem cells using 50,000 cells per well in 6-well dishes. Cells were grown in human ES medium3. The Columbia University Institutional Review Board has approved all procedures. iPS cells were cultured in MTeSR (Stemgent) on Matrigel (BD Biosciences)-coated plates and passaged according to the manufacturer's instructions.

In addition to production of iPSCs from healthy donor patients, iPSCs can be generated from samples obtained from diseased patients. For example, iPSC cell lines have been developed from T1D patients, as well as patients with monogenic and gestational diabetes (GDM) from samples obtained from the Naomi Berrie Diabetes Center. Generation of iPS cells from diseased patients can be accomplished according to published techniques (see Park I H, et al., Disease-specific induced pluripotent stem cells. Cell. 2008; 134(5):877-886; and Hua et al., J Clin Invest, 2013; 123(7):3146-3153). Human pluripotent stem cells, including iPSCs and human ES cells, have the capacity to differentiate into insulin-producing cells (Maehr R, et al. Generation of pluripotent stem cells from patients with type 1 diabetes. Proc Natl Acad Sci U S A. 2009;106(37):15768-15773.), which display key properties of β cells, including glucose-stimulated insulin secretion upon maturation in vivo (Kroon E, et al. Pancreatic endoderm derived from human embryonic stem cells generates glucose-responsive insulin-secreting cells in vivo. Nat Biotechnol. 2008;26(4):443-452.). iPSCs have been generated from patients with various types of diabetes (Park et al.; 2, Ohmine S, et al. Reprogrammed keratinocytes from elderly type 2 diabetes patients suppress senescence genes to acquire induced pluripotency. Aging (Albany N.Y.). 2012;4(1):60-73; Teo A K, et al. Derivation of human induced pluripotent stem cells from patients with maturity onset diabetes of the young. J Biol Chem. 2013;288(8):5353-5356.).

Preparation fibroblasts for production of iPSCs. Based on the Hua et al. technique, biopsies of upper arm skin are obtained from diabetic subjects or healthy subjects using local anesthesia (lidocaine) and an Acu-Punch Biopsy Kit (Acuderm Inc.). Samples are coded and transported to the laboratory. Biopsies are cut in 10 to 12 small pieces, and 2-3 pieces of minced skin are placed around a silicon droplet in a well of a 6-well dish. A glass cover slip is placed over the biopsy pieces, and 5 ml biopsy plating media was added. After 5 days, biopsy pieces are grown in culture medium for 3 to 4 weeks. Biopsy plating medium is composed of DMEM, FBS, GlutaMAX, Anti-Anti, NEAA, 2-Mercaptoethanol, and nucleosides (all from Invitrogen), and culture medium contained DMEM, FBS, GlutMAX, and Penicillin/Streptomycin (all from Invitrogen).

Expanded Protocol for Generation of iPSCs. Building on the summary provided above, primary fibroblasts are converted into pluripotent stem cells using the CytoTune-iPS Sendai Reprogramming Kit (Invitrogen). 50,000 fibroblast cells are seeded per well in a 6-well dish at passage 3 and allowed to recover overnight. Within 24-48 hours, Sendai viruses expressing human transcription factors OCT4, SOX2, Klf4, and C-Myc are mixed in fibroblast medium to infect fibroblast cells according to the manufacturer's instructions. Two days later, the medium is exchanged with human ES medium supplemented with the ALKS inhibitor SB431542 (2 μM; Stemgent), the MEK inhibitor PD0325901 (0.5 μM; Stemgent), and thiazovivin (0.5 μM; Stemgent). Human ES medium contains KO-DMEM, KSR, GlutMAX, NEAA, 2-Mercaptoethanol, Penicillin/Streptomycin, and bFGF (all from Invitrogen). On day 7-10 after infection, cells are detached using TrypLE and passaged onto feeder cells. Individual colonies of iPSCs are picked between days 21 and 28 after infection, and each iPSC line is expanded from a single colony. iPSCs lines are cultured in human ES medium. To confirm pluripotency of the iPSCs, they may be tested for teratoma potential. For example, 1-2 million cells from each iPSC line may detached and collected after TrypLE (Invitrogen) treatment. Cells are suspended in 0.5 ml human ES media. The cell suspension is mixed with 0.5 ml Matrigel (BD Biosciences) and injected subcutaneously into dorsal flanks of an immunodeficient mouse (NOD.Cg-Prkdc^{scid}Il2rg^{tmlWjl}/SzJ, stock no. 005557, The Jackson Laboratory). Eight to twelve weeks after injection, teratomas are harvested, fixed overnight with 4% paraformaldehyde, and processed according to standard procedures for paraffin embedding. The samples are then sectioned and H&E stained.

### Example 2

### CRISPR Methods and Production of Reporter iPS Cell Lines

To generate the reporter iPS cell lines, a healthy patient iPS cell line was chosen, karyotyped, and sequenced at the loci of interest. Karyotyping is done as a routine measure to be sure that the cells have a full complement of chromosomes. Guides were designed using the Optimized CRISPR Design algorithm (http://crispr.mit.edu/), and were chosen for minimal predicted off-target effects. All guides were targeted to exon 1 of the loci (target gene) of interest (Ngn, Foxo1, Tph1 or 2, and insulin). Efficiency of cutting by the guides with Cas9 protein were assessed by Surveyor assay (Transgenomic) performed in HEK-293 cells. Guides that had the most robust cutting were chosen for nucleofection (Amaxa) with Cas9-EGFP plasmid (Addgene) and the targeting vector in the patient iPS line. Human Stem Cell Nucleofector Kit 1 (Lonza) was used for the nucleofection. 10 million iPS cells split the day before were cultured on MEFs, dissociated with Accutase (Sigma), and used for nucleofection with bug of each plasmid (total 30 ug DNA). Targeting vectors were designed to introduce a fluorescent protein in exon 1 of the gene of interest, and 1 kb homology arms were used. After nucleofection, cells were sorted by FACS for GFP expression and cultured in a 10 cm dish with human ES media with Rock inhibitor on mouse embryonic fibroblasts (MEFs). After 2 weeks of culturing, individual clones were selected, split, and screened for integration of the insertion by PCR. Colonies that contained the insertion were Topoisomerase-sequenced to determine the sequence of both targeted and untargeted alleles. Clones with the desired alleles were then expanded and grown into gut organoids.

Putting the gene for the reporter in exon 1, means that it will be at the amino terminus of the fused gene ahead of the endogenous target gene. When placed in exon 1, the reporter gene comes after the promoter so that the endogenous promoter (for example for insulin) drives transcription of the reporter gene. Alternatively, the reporter gene can be positioned at the C-terminal after the endogenous target gene and before the stop codon. The promoter can drive expression of both genes. In one embodiment the reporter is fused to the target gene so that both genes are transcribed and translated together and the mRNA for both genes is in one reading frame. Another option is to make a single mRNA that is bi-cistronic, with two proteins such that one protein is made first and then the second protein is made. Theoretically, the reporter gene could be inserted anywhere, but if inserted in the middle of the endogenous gene, it will disrupt the gene.

FIG. 1 is an image of a gel demonstrating successful cutting of the guides for Foxo1 and Insulin by Surveyor Assay for the CRISPR method. FIG. 2a-d shows that insulin expression is associated with 5HT inhibition. A-D, IHC of Insulin (green), FOXO1 (red), and 5HT (white). Green arrowheads denote FOXO⁺ cells that underwent conversion to insulin⁺ cells. Note that they do NOT express 5HT (inset in C). Gray arrowheads denote FOXO⁺ cells that express 5HT. These cells did not convert into insulin⁺ cells. The white arrowhead denotes the only 5HT⁺/insulin⁺/FOXO⁺ cells identified in our experiments, also shown in the inset.

### Methods For Example 2

The nucleofection protocols provided below were used for transfection of iPS cell lines with the reporter genes. FOXO1 Nucleofection Protocol is provided as an example but the techniques were used for the other targeting constructs.

### FOXO1 Nucleofection Protocol Round 1

| | | | gRNA + Cas9 + Targeting |
|---|---|---|---|
| | Conc. | ug needed: | ul DNA |
| Foxo1 #1 gRNA | 0.4005 | 10 | 24.96878901 |
| Cas9-EGFP | 0.9396 | 10 | 10.64282673 |
| Foxo1 Targeting | 0.838 | 10 | 11.93317422 |

Before Starting: 4×6-well
a. Culture iPS to 80% confluence on 6-well plates. Will generally want 10 million per sample (˜1 6-well plate)
b. 24 hours prior to dissociation, culture cells in HuESM+Ri
c. 3 hours prior, change media again (HuESM+Ri)
d. Aliquot 500 ul HuESM+Ri in 24-well plate, with # of wells equal to the # of samples. Place at 37C. These will be used to quench the nucleofection reaction immediately after electroporation
e. Prepare eppendorfs with the appropriate amount of DNA needed for each sample (10 ug gRNA, 10 ug Cas9, 10 ug Donor). Keep on ice.

### Dissociation:

a. Aspirate media, wash 1× with PBS
b. Add 1 ml Accutase
c. Incubate for 7-12 minutes at 37C (optimal time depends on cell line. 1070 ˜6 minutes, 1083 ˜8 minutes)
d. Add 2 ml of HuESM to stop reaction
e. Collect cells into 50 ml falcon
f. Add 1 more ml HuESM in each well to collect leftovers. Add to 50 ml falcon and adjust total vol. to ˜20 ml.

### Count:

Automatic method—preferred because of speed and adjustment for dead cells
a. Mix 10 ul of Trypan Blue with 10 ul of cells
b. Place 10 ul onto each side of the chamber on the cell counter slide
c. Insert slide into the Countess machine in Leibel lab.
d. Output will be total number of cells, dead and alive.
e. Calculate the number of cells you have total and aliquot out the correct vol. for 10 million cells. *** Point at which you use samples 1 at a time.
f. Spin down at 800 rpm for 5 min. at RT (should have 4 tubes), remove supernatant.
Keep cells on ice

Nucleofection: 4× 6-well
a. Resuspend cells in 82 ul Nucleofection solution+18 ul Supplement (4.5:1 ratio)
b. Pipette 100 ul of cells in nucleofection solution into chilled tubes containing the DNA (4 tubes)
c. Mix and transfer to cuvette
d. Run program A23
e. Immediately add 500 ul of the pre-warmed HuESM+Ri from the 24-well plate.
f. Aspirate media from a 6-well plate of MEFs
g. Using dropper, distribute across the 6-well plate of MEFs
h. Top up with 1.5 ml HuESM+Ri to a total vol. of 2 ml
i. Repeat for other samples
j. When finished, store cells at 37C

### Culturing:

a. Next day (D2), change media with HuESM+Ri
b. On D3, prepare for FACS.

### Sorting: 4×10 cm

a. 2.5 hr before sorting, change media to HuESM +RI (***Including non-transfected ctrl, ˜20mL)
b. 1.5 hr before sorting, dissociate with Accutase (˜5 min. at 37C)
c. Collect each well with 3 ml of normal HuES media in 15 ml falcon tubes
d. Spin down, wash once with HuESM
e. Resuspend in 2 ml HuESM+RI (˜20 mL)
f. Dissociate by triturating 20× with a 1 ml pipette.
g. Filter cells through a 30um blue filter (cap of the sorting tube, use unopened pack)
h. Put on the actual cap
i. Spin down 1 more time and resuspend in 300-500 ul HuESM+RI+AntiAnti
j. Prepare 4-6 tubes to sort into, containing media with Anti-Anti (100×) and no P/S
k. SORT
l. After sorting, plate on 10 cm dish for easier picking.
   FOXO1 Nucleofection protocol Round 2

| | | | gRNA + Cas9 + Targeting |
|---|---|---|---|
| | Conc. | ug needed: | ul DNA |
| Foxo1 #1 gRNA | 0.4005 | 10 | 24.96878901 |
| Cas9-EGFP | 0.9396 | 10 | 10.64282673 |
| Foxo1 Targeting | 0.6 | 10 | 16.66666667 |

Before Starting: 4× 6-well
a. Culture iPS to 80% confluence on 6-well plates. Will generally want 10million per sample (˜1 6-well plate)
b. 24 hours prior to dissociation, culture cells in HuESM+Ri
c. 3 hours prior, change media again (HuESM+Ri)
d. Aliquot 500 ul HuESM+Ri in 24-well plate, with # of wells equal to the # of samples. Place at 37C. These will be used to quench the nucleofection reaction immediately after electroporation e. Prepare eppendorfs with the appropriate amount of DNA needed for each sample (10 ug gRNA, 10 ug Cas9, 10 ug Donor). Keep on ice.

### Dissociation:

a. Aspirate media, wash 1× with PBS
b. Add 1 ml Accutase
c. Incubate for 7-12 minutes at 37C (optimal time depends on cell line. 1070 ˜6 minutes, 1083 ˜8 minutes)
d. Add 2 ml of HuESM to stop reaction
e. Collect cells into 50 ml falcon
f. Add 1 more ml HuESM in each well to collect leftovers. Add to 50 ml falcon and adjust total vol. to ˜20 ml.
   Count: Automatic method—preferred because of speed and adjustment for dead cells
a. Mix 10 ul of Trypan Blue with 10 ul of cells
b. Place 10 ul onto each side of the chamber on the cell counter slide
c. Insert slide into the Countess machine in Leibel lab.
d. Output will be total number of cells, dead and alive.
e. Calculate the number of cells you have total and aliquot out the correct vol. for 10 million cells. *** Point at which you use samples 1 at a time. Keep cells on ice.
f. Spin down at 800 rpm for 5 min. at RT (should have 4 tubes), remove supernatant.
   Nucleofection: 4× 6-well
a. Resuspend cells in 82 ul Nucleofection solution+18 ul Supplement (4.5:1 ratio)
b. Pipette 100 ul of cells in nucleofection solution into chilled tubes containing the DNA (4 tubes)
c. Mix and transfer to cuvette
d. Run program A23
e. Immediately add 500 ul of the pre-warmed HuESM+Ri from the 24-well plate.
   f. Aspirate media from a 6-well plate of MEFs
g. Using dropper, distribute across the 6-well plate of MEFs
h. Top up with 1.5 ml HuESM+Ri to a total vol. of 2 ml
i. Repeat for other samples
j. When finished, store cells at 37C

### Culturing:

a. Next day (D2), change media with HuESM+Ri
b. On D3, prepare for FACS.

### Sorting: 4× 10cm

a. 2.5 hr before sorting, change media to HuESM+RI (***Including non-transfected ctrl, ˜20 mL)
b. 1.5 hr before sorting, dissociate with Accutase (˜5 min. at 37C)
c. Collect each well with 3 ml of normal HuES media in 15 ml falcon tubes
d. Spin down, wash once with HuESM
e. Resuspend in 2 ml HuESM+RI (˜20 mL)
f. Dissociate by triturating 20× with a 1 ml pipette.
g. Filter cells through a 30um blue filter (cap of the sorting tube, use unopened pack)
h. Put on the actual cap
i. Spin down 1 more time and resuspend in 300-500 ul HuESM+RI+AntiAnti
   j. Prepare 4-6 tubes to sort into, containing media with Anti-Anti (100×) and no P/S
   k. SORT
l. After sorting, plate on 10 cm dish for easier picking.
   Added Gentamicin 50 ug/mL next day for ˜8 hrs, then switched back to Hri

### Targeting Vector Sequences

The following Target Vector Sequences were used for nucleofection of iPS cells to create reporter cell lines for Ngn3, Tph2, and Foxo 1.

Ngn3-EGFP-pA-Ngn3 1083 1 Kb Arms
tcgcgcgtttcggtgatgacggtgaaaacctctgacacatgcagctcccggagacggtcacagcttgtctgtaag
cggatgccgggagcagacaagcccgtcagggcgcgtcagcgggtgttggcgggtgtcggggctggcttaactatg
cggcatcagagcagattgtactgagagtgcaccatatgcggtgtgaaataccgcacagatgcgtaaggagaaaat
accgcatcaggcgccattcgccattcaggctgcgcaactgttgggaagggcgatcggtgcgggcctcttcgctat
tacgccagctggcgaaagggggatgtgctgcaaggcgattaagttgggtaacgccagggttttcccagtcacgac
gttgtaaaacgacggccagtgaattcgagctcggtacctcgcgaatgcatctagacagacagacttgagtgaggg
tagggcgacccaagacggtgggcggctccggccgggtagtgctaccattctagtattctttgaatgagattatgg
ggtggtggcagagaggaggcctaaaatgagcgcactttgcaatgcccacttcgcgcgggcagcagcaagggttgc
gtgcgttggcgcggctcggagggccggggaatgaacccagcctgccgcccccgtggaggcctgggccggccaggg
gtcagccagggagaagcagaaggaacaagtgcttttgagggccgccgccgtcggccaccctctacggctcccggc
tccctccctctcccttacccttagcacccacagcccagcgacagacaggtcctttcacagaaaatctcgagaaag
ccagactgcctgggctcaagcaggcggaagaggtggcccccagcagcccgggtcgctcctccagcgacgcggcgg
gactcaggctgccagcctgggagactggggagtagagggacccccagtccccgggggaaccgcctgggctgccca
gctccccgcagtgcggcgccggcggctccagcgcgtacaagctgtggtccgctatgcgcagcgtttgagtcagcg
cccagatgtagttgtgggcgaagcgcagcgtctcgatcttggtgagcttcgcgtcgtctgggaaggtgggcagga
caccgcgcagggcgtccagtgccgagttgaggttgtgcattcgattgcgctcgcggtcgttggccttctttcgcc
gactccgtcgctgcttgctcagtgccaactcgctcttaggccggctgcgtcccccgcgccgtgcccggagcttcc
tcggggcccctcggcagcctccctcttccgcctctgcgcagttcccccgtgtgcgagtggggctgggcggggcgg
acgtggggcaggtcacttcgtcttccgaggctctggggaaggaccgctccgtctcacgggtcacttggacagtgg
gcgcacccatagagcccaccgcatccccagcatgcctgctattgtcttcccaatcctcccccttgctgtcctgcc
ccaccccaccccccagaatagaatgacacctactcagacaatgcgatgcaatttcctcattttattaggaaagga
cagtgggagtggcaccttccagggtcaaggaaggcacgggggaggggcaaacaacagatggctggcaactagtca
cttgtacagctcgtccatgccgagagtgatcccggcggcggtcacgaactccagcaggaccatgtgatcgcgctt
ctcgttggggtctttgctcagggcggactgggtgctcaggtagtggttgtcgggcagcagcacggggccgtcgcc
gatgggggtgttctgctggtagtggtcggcgagctgcacgctgccgtcctcgatgttgtggcggatcttgaagtt
caccttgatgccgttcttctgcttgtcggccatgatatagacgttgtggctgttgtagttgtactccagcttgtg
ccccaggatgttgccgtcctccttgaagtcgatgcccttcagctcgatgcggttcaccagggtgtcgccctcgaa
cttcacctcggcgcgggtcttgtagttgccgtcgtccttgaagaagatggtgcgctcctggacgtagccttcggg
catggcggacttgaagaagtcgtgctgcttcatgtggtcggggtagcggctgaagcactgcacgccgtaggtcag
ggtggtcacgagggtgggccagggcacgggcagcttgccggtggtgcagatgaacttcagggtcagcttgccgta
ggtggcatcgccctcgccctcgccggacacgctgaacttgtggccgtttacgtcgccgtccagctcgaccaggat
gggcaccaccccggtgaacagctcctcgcccttgctcaccatccgagggttgaggcgtcatcctacggcggggtc
agagggaagggtaagtttgagtccgtcactgggcgcagtccgcgattccgaggctaggtgggaaaaaacaaaaac
agccatcctcccagcccccgctgggtcagaggatccctctttcccctgcccgtccctcggaggcctccaaatatt
acctttctaccggcgcaaaagaatagagagcgatgagcagcgagggccgtggggagctcagcgggcttctggtcg
ccaagttcagctgagctgcaggcgcccccgcctgggagttgccccagccccaaaggagaaaagaagagagaatgg
ggtccgaggcctctgtcacgctctctctcgaggcgcggcggtgagaccgcagggatttcctgagcagcaagtcgt
gtgccccttggcacgctttatctgcttcgcccgggccaggagcgtgcctgcccggctgctgcccgcgccaccggc
caatcagcgccggggccctggggccgcgccacgcgagcccgctcctcccccgcagggcacagctggattccggac
aaagggccggggtcgggggaggggagcgccgctctgtttgctctctcgagggcgggctgggtcccagcaactctc
ggttcctcaaagagcctcgcccagtgagaagagcctcgtgtggctctggtcaggccacctcagacggctttgctc
ctagcctatctttccttagcatctgtcctggaggggactttgatgcctctagggtacaatgcctgcacgttacac
atggggaaatttaggcttagtgagggaggtggcttgtctgaaatcgcacaggaagatagtggcaaagacaaccac
gagctcattgtcctgactagcagcctggagaagggtccaggaattctaaaggacgccctgctctcctggtgtttc
actgcctctcttcatcctggaagacaggggacatcactgagagagatcctgcctatgtcccttccattgtcgact
gcagaggcctgcatgcaagcttggcgtaatcatggtcatagctgtttcctgtgtgaaattgttatccgctcacaa
ttccacacaacatacgagccggaagcataaagtgtaaagcctggggtgcctaatgagtgagctaactcacattaa
ttgcgttgcgctcactgcccgctttccagtcgggaaacctgtcgtgccagctgcattaatgaatcggccaacgcg
cggggagaggcggtttgcgtattgggcgctcttccgcttcctcgctcactgactcgctgcgctcggtcgttcggc
tgcggcgagcggtatcagctcactcaaaggcggtaatacggttatccacagaatcaggggataacgcaggaaaga
acatgtgagcaaaaggccagcaaaaggccaggaaccgtaaaaaggccgcgttgctggcgtttttccataggctcc
gcccccctgacgagcatcacaaaaatcgacgctcaagtcagaggtggcgaaacccgacaggactataaagatacc
aggcgtttccccctggaagctccctcgtgcgctctcctgttccgaccctgccgcttaccggatacctgtccgcct
ttctcccttcgggaagcgtggcgctttctcatagctcacgctgtaggtatctcagttcggtgtaggtcgttcgct
ccaagctgggctgtgtgcacgaaccccccgttcagcccgaccgctgcgccttatccggtaactatcgtcttgagt
ccaacccggtaagacacgacttatcgccactggcagcagccactggtaacaggattagcagagcgaggtatgtag
gcggtgctacagagttcttgaagtggtggcctaactacggctacactagaagaacagtatttggtatctgcgctc
tgctgaagccagttaccttcggaaaaagagttggtagctcttgatccggcaaacaaaccaccgctggtagcggtg
gtttttttgtttgcaagcagcagattacgcgcagaaaaaaaggatctcaagaagatcctttgatcttttctacgg
ggtctgacgctcagtggaacgaaaactcacgttaagggattttggtcatgagattatcaaaaaggatcttcacct
agatccttttaaattaaaaatgaagttttaaatcaatctaaagtatatatgagtaaacttggtctgacagttacc
aatgcttaatcagtgaggcacctatctcagcgatctgtctatttcgttcatccatagttgcctgactccccgtcg
tgtagataactacgatacgggagggcttaccatctggccccagtgctgcaatgataccgcgagacccacgctcac
cggctccagatttatcagcaataaaccagccagccggaagggccgagcgcagaagtggtcctgcaactttatccg
cctccatccagtctattaattgttgccgggaagctagagtaagtagttcgccagttaatagtttgcgcaacgttg
ttgccattgctacaggcatcgtggtgtcacgctcgtcgtttggtatggcttcattcagctccggttcccaacgat
caaggcgagttacatgatcccccatgttgtgcaaaaaagcggttagctccttcggtcctccgatcgttgtcagaa
gtaagttggccgcagtgttatcactcatggttatggcagcactgcataattctcttactgtcatgccatccgtaa
gatgcttttctgtgactggtgagtactcaaccaagtcattctgagaatagtgtatgcggcgaccgagttgctctt
gcccggcgtcaatacgggataataccgcgccacatagcagaactttaaaagtgctcatcattggaaaacgttctt
cggggcgaaaactctcaaggatcttaccgctgttgagatccagttcgatgtaacccactcgtgcacccaactgat
cttcagcatcttttactttcaccagcgtttctgggtgagcaaaaacaggaaggcaaaatgccgcaaaaaagggaa
taagggcgacacggaaatgttgaatactcatactcttcctttttcaatattattgaagcatttatcagggttatt
gtctcatgagcggatacatatttgaatgtatttagaaaaataaacaaataggggttccgcgcacatttccccgaa
aagtgccacctgacgtctaagaaaccattattatcatgacattaacctataaaaataggcgtatcacgaggccct
ttcgtc
Tph2-Cerulean-pA-Tph2 1083 1 Kb Arms
tcgcgcgtttcggtgatgacggtgaaaacctctgacacatgcagctcccggagacggtcacagcttgtctgtaag
cggatgccgggagcagacaagcccgtcagggcgcgtcagcgggtgttggcgggtgtcggggctggcttaactatg
cggcatcagagcagattgtactgagagtgcaccatatgcggtgtgaaataccgcacagatgcgtaaggagaaaat
accgcatcaggcgccattcgccattcaggctgcgcaactgttgggaagggcgatcggtgcgggcctcttcgctat
tacgccagctggcgaaagggggatgtgctgcaaggcgattaagttgggtaacgccagggttttcccagtcacgac
gttgtaaaacgacggccagtgaattcgagctcggtacctcgcgaatgcatctagatccagtgaattcgagctcgg
tacctcgcgaatgcatctagacctttcctttgcaatacattttcctccatataactctgcatagaggcatcacag
gattaagaagaagcccttttatgaaagccattacacatatatacactcacacatttgcatgcacaaaattagaat
atgtcaagtcagaaaaagcttattaacataaaatggagttggtcaatgagtaaaaaaaatatgctgatgggaggg
ataagatctagtgttcgggagcacaataatttattttcttttgtattttaaaataactggaagagtggaattgga
atgtttctaacacaaaaagaaatgataaatgcttgaggcaatggatatcttgattaccttatttgatcattacac
attgtacgcttgtgtcaaaatatcacatgtgccttataaatgtgtacaactattagttatccataaaaattaaaa
attaaaaaatccgtaaaatggtttaagcattcagcagtgctgatctttcttaaattatttttctaattttggaaa
gaaagcacaaaatctttgaattcacaattgcttaaagactgaggttaacttgccagtggcaggcttgagagatga
gagaactaacgtcagaggatagatggtttcttgtacaaataacacccccttatgtattgttctccaccacccccg
cccaaaaagctactcgacctatgaaacaaatcacactatgagcacagataaccccaggcttcaggtctgtaatct
gactgtggccatcggcaaccagaaatgagtttctttctaatcagtcttgcatcagtctccagtcattcatataaa
ggagcccggggatgggaggattcgcattgctcttcagcaccagggttctggacagcgccccaagcaggcagctga
tcgcacgccccttcctctcaatctccgccagcgctgctactgcccctctagtaccccctgctgcagagaaagaat
attacaccgggatccatgcagccagcaatgatgatgttttccagtaaatactgggcacggatggtgagcaagggc
gaggagctgttcaccggggtggtgcccatcctggtcgagctggacggcgacgtaaacggccacaagttcagcgtg
tccggcgagggcgagggcgatgccacctacggcaagctgaccctgaagttcatctgcaccaccggcaagctgccc
gtgccctggcccaccctcgtgaccaccctgacctggggcgtgcagtgcttcgcccgctaccccgaccacatgaag
cagcacgacttcttcaagtccgccatgcccgaaggctacgtccaggagcgcaccatcttcttcaaggacgacggc
aactacaagacccgcgccgaggtgaagttcgagggcgacaccctggtgaaccgcatcgagctgaagggcatcgac
ttcaaggaggacggcaacatcctggggcacaagctggagtacaacgccatcagcgacaacgtctatatcaccgcc
gacaagcagaagaacggcatcaaggccaacttcaagatccgccacaacatcgaggacggcagcgtgcagctcgcc
gaccactaccagcagaacacccccatcggcgacggccccgtgctgctgcccgacaaccactacctgagcacccag
tccgccctgagcaaagaccccaacgagaagcgcgatcacatggtcctgctggagttcgtgaccgccgccgggatc
actctcggcatggacgagctgtacaagtgactagttgccagccatctgttgtttgcccctcccccgtgccttcct
tgaccctggaaggtgccactcccactgtcctttcctaataaaatgaggaaattgcatcgcattgtctgagtaggt
gtcattctattctggggggtggggtggggcaggacagcaagggggaggattgggaagacaatagcaggcatgctg
gggatgcggtgggctctatggagagggttttccctggattcagcagtgcccgaagagcatcagctacttggcagc
tcaacagtgagtactacgtacctggcactatggagaattattttttagggtgtgaccatcttctcctcaccatat
gaatcccttttgtagtgtaagcacgcacacctcaaatttctccttctttataatctgtctaccctgctttcctcc
tgtctgcctccagtcttcctcttctctccataagtaaagcgagtgtgccaatcactgcgtgctcaactttttttc
cgcaaagtttgtaagtagagagttaagaagttcctgaacattaagaatgagagattgtatgaatcaatgtcttaa
atctacagccaaaaaaaaaaaaaaaaaaatggagtgtgaagaattttgaaaagccgtttattatgaggaggagga
gtagggagaacaaattaaataaatttccacggttttcagaagatcattgtgtctcctacacccccttcagtttac
aaagcctggtctttaaacatagaactattattttctcttcttagttatgggtgcaggttattggaataaaagaaa
gattggattcctttcaaaagtttttctgtgtttcacattgctcaatttttttcagtttacttgatggaataatga
aagcaatacaccacttgctatagtatttaagggagttttatgtttataatatctacaggataaaaaagcagtatt
tgcaggattttagatcctgctttcaggtagtagtcatgggatttaataaaaaccacgaaataaaaatgtatccag
gtcctagtcattaaaaatattaaatggtattttattactgtactatcagagtttatcaaccaaatccaattcagt
ctgtatcatagaatcatctgttttaatttcgtagctccaaatatgtgccagagggctgcgttggactgacatatt
attactgataaaaatgttgaaaagtaaacatggcaacttctgtagagtcgactgcagaggcctgcatgcaagctt
ggcgtaatcatcggatcccgggcccgtcgactgcagaggcctgcatgcaagcttggcgtaatcatggtcatagct
gtttcctgtgtgaaattgttatccgctcacaattccacacaacatacgagccggaagcataaagtgtaaagcctg
gggtgcctaatgagtgagctaactcacattaattgcgttgcgctcactgcccgctttccagtcgggaaacctgtc
gtgccagctgcattaatgaatcggccaacgcgcggggagaggcggtttgcgtattgggcgctcttccgcttcctc
gctcactgactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaaccgtaaaaa
ggccgcgttgctggcgtttttccataggctccgcccccctgacgagcatcacaaaaatcgacgctcaagtcagag
gtggcgaaacccgacaggactataaagataccaggcgtttccccctggaagctccctcgtgcgctctcctgttcc
gaccctgccgcttaccggatacctgtccgcctttctcccttcgggaagcgtggcgctttctcatagctcacgctg
taggtatctcagttcggtgtaggtcgttcgctccaagctgggctgtgtgcacgaaccccccgttcagcccgaccg
ctgcgccttatccggtaactatcgtcttgagtccaacccggtaagacacgacttatcgccactggcagcagccac
tggtaacaggattagcagagcgaggtatgtaggcggtgctacagagttcttgaagtggtggcctaactacggcta
cactagaagaacagtatttggtatctgcgctctgctgaagccagttaccttcggaaaaagagttggtagctcttg
atccggcaaacaaaccaccgctggtagcggtggtttttttgtttgcaagcagcagattacgcgcagaaaaaaagg
atctcaagaagatcctttgatcttttctacggggtctgacgctcagtggaacgaaaactcacgttaagggatttt
ggtcatgagattatcaaaaaggatcttcacctagatccttttaaattaaaaatgaagttttaaatcaatctaaag
tatatatgagtaaacttggtctgacagttaccaatgcttaatcagtgaggcacctatctcagcgatctgtctatt
tcgttcatccatagttgcctgactccccgtcgtgtagataactacgatacgggagggcttaccatctggccccag
tgctgcaatgataccgcgagacccacgctcaccggctccagatttatcagcaataaaccagccagccggaagggc
cgagcgcagaagtggtcctgcaactttatccgcctccatccagtctattaattgttgccgggaagctagagtaag
tagttcgccagttaatagtttgcgcaacgttgttgccattgctacaggcatcgtggtgtcacgctcgtcgtttgg
tatggcttcattcagctccggttcccaacgatcaaggcgagttacatgatcccccatgttgtgcaaaaaagcggt
tagctccttcggtcctccgatcgttgtcagaagtaagttggccgcagtgttatcactcatggttatggcagcact
gcataattctcttactgtcatgccatccgtaagatgcttttctgtgactggtgagtactcaaccaagtcattctg
agaatagtgtatgcggcgaccgagttgctcttgcccggcgtcaatacgggataataccgcgccacatagcagaac
tttaaaagtgctcatcattggaaaacgttcttcggggcgaaaactctcaaggatcttaccgctgttgagatccag
ttcgatgtaacccactcgtgcacccaactgatcttcagcatcttttactttcaccagcgtttctgggtgagcaaa
aacaggaaggcaaaatgccgcaaaaaagggaataagggcgacacggaaatgttgaatactcatactcttcctttt
tcaatattattgaagcatttatcagggttattgtctcatgagcggatacatatttgaatgtatttagaaaaataa
acaaataggggttccgcgcacatttccccgaaaagtgccacctgacgtctaagaaaccattattatcatgacatt
aacctataaaaataggcgtatcacgaggccctttcgtc
Foxo1-mOrange-pA-Foxo1 1083 1 Kb Arms
tcgcgcgtttcggtgatgacggtgaaaacctctgacacatgcagctcccggagacggtcacagcttgtctgtaag
cggatgccgggagcagacaagcccgtcagggcgcgtcagcgggtgttggcgggtgtcggggctggcttaactatg
cggcatcagagcagattgtactgagagtgcaccatatgcggtgtgaaataccgcacagatgcgtaaggagaaaat
accgcatcaggcgccattcgccattcaggctgcgcaactgttgggaagggcgatcggtgcgggcctcttcgctat
tacgccagctggcgaaagggggatgtgctgcaaggcgattaagttgggtaacgccagggttttcccagtcacgac
gttgtaaaacgacggccagtgaattcgagctcggtacctcgcgaatgcatctagaagagaacccgccctcccccc
gcggaggtccgggagggaaggggcagccgaagcagtcggcgcgggccgggggttgccgctcccagcgaacccctt
tctcctttcactggcaaacttttcggcctcgctctgacgtccacttcttggcgcactttctttacttagttcccc
aacgagccccttaccgcgtcccacgcgaactcctgactggcgcgcacgcacacctactgccgtccccgaccggac
ccgggcgaggccaccgcgaccaccgcttctcgcccgccctcctgggaacgcgctgccctcctgctccgcaccttc
aggccgagcaaacctgcacagctgcgccctcgcctgacccaccgcgcccccaaggtccggccgcgcgccgagtcc
actcaccttccagcccgccgagctgttgctgtcacccttatccttgaagtagggcacgctcttgaccatccactc
gtagatctgcgacagcgtgagccgcttctccgccgagctctcgatggccttggtgatgaggtcggcgtaggacag
gttgccccacgcgttgcggcgggacgagctgctcttgcgcggctgccccgcgagcggcccagcggcggcgggggg
caccggcgggtgctgcgacagcggcccgggcggcgggggctgcggtggcgctgggtgcaggcagcccgcctccgg
gccctggaagtccccgcacagccccccggtggcggccgcggcggccgccgccgccaccgccgccgccacggagcc
gggcgcctgcgggaagtcctcgctctcctccagcaagctcaggttgctcatgaagtcggcgctgacagcggcagc
cgaggccgagggcaggcccgccgcggcgtcggggttggcagccgcgctgcccgacggcgccgggctggaggtggc
cgagttggactggctaaactccggcctgggcagcggccaggtgcacgagcgcggccggggcagcggctcgaagtc
cgggtccatagagcccaccgcatccccagcatgcctgctattgtcttcccaatcctcccccttgctgtcctgccc
caccccaccccccagaatagaatgacacctactcagacaatgcgatgcaatttcctcattttattaggaaaggac
agtgggagtggcaccttccagggtcaaggaaggcacgggggaggggcaaacaacagatggctggcaactagctac
ttgtacagctcgtccatgccgccggtggagtggcggccctcggcgcgttcgtactgttccacgatggtgtagtcc
tcgttgtgggaggtgatgtccaacttgatgccgacgatgtaggcgccgggcagctgcacgggcttcttggccttg
taggtggtcttgacctcggaggtgtagtggccgccgtccttcagcttcagcctcatcttgatctcgcccttcagg
gcgccgtcctcggggtacatccgctcggaggaggcctcccagcccatggtcttcttctgcattacggggccgtcg
gaggggaagttggtgccgcgcagcttcaccttgtagatgaactcgccgtcctggagggaggagtcctgggtcacg
gtcaccacgccgccgtcctcgaagttcatcacgcgctcccacttgaagccctcggggaaggacagcttgaagtag
tcggggatgtcggcggggtgcttcacgtaggccttggagccgtaggtgaactgaggggacaggatgtcccaggcg
aagggcagggggccacccttggtcaccttcagcttagcggtctgaaagccctcgtaggggcggccctcgccctcg
ccctcgatctcgaactcgtggccgttcacggagccctccatgcgcaccttgaagcgcatgaactccttgatgatg
gccatgttattctcctcgcccttgctcaccatcgatctccaccacctgaggcgcctcggccatggtgacccccgc
ccctcccccagccgcaggagagccaagagggggagaacgcagcactgggggcggacggggagggggcgcgaaggg
acggtccgagatttgggggaacgaagccggtgcggcgagcggacggaaactgggaggaaggcgcggcggagtgga
agcgcgagcccagaacttaacttcgcggggccatccacatcgaggctcctcggggtccgccgcacggactggacg
gccggccagagccgccgggccggggcagagcctgcgccgcgctccagctgacagggccgcggacggaaggacgga
cggacgccgcgggccgcttgctctccccagcggcgcgcccgctgcgctgctgcctgttgaatgtggcggctgcgg
cagcggctgctgcgactaccaggccgcccgacttacgggatctgccgccgccccccgcccgcggcggcgcgcgcg
ccggcccgcccctgaccgacagcccgcgcggccaatgggcatgcggcaccgccgcccgggcagccagtgggcgcc
gggctgggtggggcccggttttccacggggaggcggcggtgggctggtggggggtagtggggtgtttttctcttt
cacacactcacctcctttttttttttttggatctctattattttctggtaattctcgagtgtttctgtgattctc
tcgccttctcagtgttttgattgctaggaagcaaaccagcgtggaggcgccggcgacactttgtttactacggag
cagcagagccgagtactcgggaagcccgggtgggaggaggcgctcgctgctccctgacctccgctgcgggccgag
cccggcgggctggcagggcagggggccgagggccgggggcgcggggtgggcgggcggaggcggccgcgaggaatt
ctactcaatcgctccctcctggctccacccacgatgtctttgctgaacgacgtggggaagtcgactgcagaggcc
tgcatgcaagcttggcgtaatcatggtcatagctgtttcctgtgtgaaattgttatccgctcacaattccacaca
acatacgagccggaagcataaagtgtaaagcctggggtgcctaatgagtgagctaactcacattaattgcgttgc
gctcactgcccgctttccagtcgggaaacctgtcgtgccagctgcattaatgaatcggccaacgcgcggggagag
gcggtttgcgtattgggcgctcttccgcttcctcgctcactgactcgctgcgctcggtcgttcggctgcggcgag
cggtatcagctcactcaaaggcggtaatacggttatccacagaatcaggggataacgcaggaaagaacatgtgag
caaaaggccagcaaaaggccaggaaccgtaaaaaggccgcgttgctggcgtttttccataggctccgcccccctg
acgagcatcacaaaaatcgacgctcaagtcagaggtggcgaaacccgacaggactataaagataccaggcgtttc
cccctggaagctccctcgtgcgctctcctgttccgaccctgccgcttaccggatacctgtccgcctttctccctt
cgggaagcgtggcgctttctcatagctcacgctgtaggtatctcagttcggtgtaggtcgttcgctccaagctgg
gctgtgtgcacgaaccccccgttcagcccgaccgctgcgccttatccggtaactatcgtcttgagtccaacccgg
taagacacgacttatcgccactggcagcagccactggtaacaggattagcagagcgaggtatgtaggcggtgcta
cagagttcttgaagtggtggcctaactacggctacactagaagaacagtatttggtatctgcgctctgctgaagc
cagttaccttcggaaaaagagttggtagctcttgatccggcaaacaaaccaccgctggtagcggtggtttttttg
tttgcaagcagcagattacgcgcagaaaaaaaggatctcaagaagatcctttgatcttttctacggggtctgacg
ctcagtggaacgaaaactcacgttaagggattttggtcatgagattatcaaaaaggatcttcacctagatccttt
taaattaaaaatgaagttttaaatcaatctaaagtatatatgagtaaacttggtctgacagttaccaatgcttaa
tcagtgaggcacctatctcagcgatctgtctatttcgttcatccatagttgcctgactccccgtcgtgtagataa
ctacgatacgggagggcttaccatctggccccagtgctgcaatgataccgcgagacccacgctcaccggctccag
atttatcagcaataaaccagccagccggaagggccgagcgcagaagtggtcctgcaactttatccgcctccatcc
agtctattaattgttgccgggaagctagagtaagtagttcgccagttaatagtttgcgcaacgttgttgccattg
ctacaggcatcgtggtgtcacgctcgtcgtttggtatggcttcattcagctccggttcccaacgatcaaggcgag
ttacatgatcccccatgttgtgcaaaaaagcggttagctccttcggtcctccgatcgttgtcagaagtaagttgg
ccgcagtgttatcactcatggttatggcagcactgcataattctcttactgtcatgccatccgtaagatgctttt
ctgtgactggtgagtactcaaccaagtcattctgagaatagtgtatgcggcgaccgagttgctcttgcccggcgt
caatacgggataataccgcgccacatagcagaactttaaaagtgctcatcattggaaaacgttcttcggggcgaa
aactctcaaggatcttaccgctgttgagatccagttcgatgtaacccactcgtgcacccaactgatcttcagcat
cttttactttcaccagcgtttctgggtgagcaaaaacaggaaggcaaaatgccgcaaaaaagggaataagggcga
cacggaaatgttgaatactcatactcttcctttttcaatattattgaagcatttatcagggttattgtctcatga
gcggatacatatttgaatgtatttagaaaaataaacaaataggggttccgcgcacatttccccgaaaagtgccac
ctgacgtctaagaaaccattattatcatgacattaacctataaaaataggcgtatcacgaggccctttcgtc
pUC57 Backbone Sequence for the Targeting Vectors
tcgcgcgtttcggtgatgacggtgaaaacctctgacacatgcagctcccggagacggtcacagcttgtctgtaag
cggatgccgggagcagacaagcccgtcagggcgcgtcagcgggtgttggcgggtgtcggggctggcttaactatg
cggcatcagagcagattgtactgagagtgcaccatatgcggtgtgaaataccgcacagatgcgtaaggagaaaat
accgcatcaggcgccattcgccattcaggctgcgcaactgttgggaagggcgatcggtgcgggcctcttcgctat
tacgccagctggcgaaagggggatgtgctgcaaggcgattaagttgggtaacgccagggttttcccagtcacgac
gttgtaaaacgacggccagtgaattcgagctcggtacctcgcgaatgcatctagatatcggatcccgggcccgtc
gactgcagaggcctgcatgcaagcttggcgtaatcatggtcatagctgtttcctgtgtgaaattgttatccgctc
acaattccacacaacatacgagccggaagcataaagtgtaaagcctggggtgcctaatgagtgagctaactcaca
ttaattgcgttgcgctcactgcccgctttccagtcgggaaacctgtcgtgccagctgcattaatgaatcggccaa
cgcgcggggagaggcggtttgcgtattgggcgctcttccgcttcctcgctcactgactcgctgcgctcggtcgtt
cggctgcggcgagcggtatcagctcactcaaaggcggtaatacggttatccacagaatcaggggataacgcagga
aagaacatgtgagcaaaaggccagcaaaaggccaggaaccgtaaaaaggccgcgttgctggcgtttttccatagg
ctccgcccccctgacgagcatcacaaaaatcgacgctcaagtcagaggtggcgaaacccgacaggactataaaga
taccaggcgtttccccctggaagctccctcgtgcgctctcctgttccgaccctgccgcttaccggatacctgtcc
gcctttctcccttcgggaagcgtggcgctttctcatagctcacgctgtaggtatctcagttcggtgtaggtcgtt
cgctccaagctgggctgtgtgcacgaaccccccgttcagcccgaccgctgcgccttatccggtaactatcgtctt
gagtccaacccggtaagacacgacttatcgccactggcagcagccactggtaacaggattagcagagcgaggtat
gtaggcggtgctacagagttcttgaagtggtggcctaactacggctacactagaagaacagtatttggtatctgc
gctctgctgaagccagttaccttcggaaaaagagttggtagctcttgatccggcaaacaaaccaccgctggtagc
ggtggtttttttgtttgcaagcagcagattacgcgcagaaaaaaaggatctcaagaagatcctttgatcttttct
acggggtctgacgctcagtggaacgaaaactcacgttaagggattttggtcatgagattatcaaaaaggatcttc
acctagatccttttaaattaaaaatgaagttttaaatcaatctaaagtatatatgagtaaacttggtctgacagt
taccaatgcttaatcagtgaggcacctatctcagcgatctgtctatttcgttcatccatagttgcctgactcccc
gtcgtgtagataactacgatacgggagggcttaccatctggccccagtgctgcaatgataccgcgagacccacgc
tcaccggctccagatttatcagcaataaaccagccagccggaagggccgagcgcagaagtggtcctgcaacttta
tccgcctccatccagtctattaattgttgccgggaagctagagtaagtagttcgccagttaatagtttgcgcaac
gttgttgccattgctacaggcatcgtggtgtcacgctcgtcgtttggtatggcttcattcagctccggttcccaa
cgatcaaggcgagttacatgatcccccatgttgtgcaaaaaagcggttagctccttcggtcctccgatcgttgtc
agaagtaagttggccgcagtgttatcactcatggttatggcagcactgcataattctcttactgtcatgccatcc
gtaagatgcttttctgtgactggtgagtactcaaccaagtcattctgagaatagtgtatgcggcgaccgagttgc
tcttgcccggcgtcaatacgggataataccgcgccacatagcagaactttaaaagtgctcatcattggaaaacgt
tcttcggggcgaaaactctcaaggatcttaccgctgttgagatccagttcgatgtaacccactcgtgcacccaac
tgatcttcagcatcttttactttcaccagcgtttctgggtgagcaaaaacaggaaggcaaaatgccgcaaaaaag
ggaataagggcgacacggaaatgttgaatactcatactcttcctttttcaatattattgaagcatttatcagggt
tattgtctcatgagcggatacatatttgaatgtatttagaaaaataaacaaataggggttccgcgcacatttccc
cgaaaagtgccacctgacgtctaagaaaccattattatcatgacattaacctataaaaataggcgtatcacgagg
ccctttcgtc
Insulin

The insulin-GFP human ES line was generated by E. G. Stanley as described in Micallef et al. INS^{GFP/w} human embryonic stem cells facilitate isolation of in vitro derived insulin-producing cells; Diabetologia, 2012, 55(3):694-706 by conventional homologous recombination.

| | |
|---|---|
| Ngn3 gRNA #4 | TGGACAGTGGGCGCACCCG |
| Ngn3 gRNA #8 | GGACAGTGGGCGCACCCGA |
| Foxo1 gRNA #1 | CAGGTGGTGGAGATCGACC |
| Foxo1 gRNA #10 | ACCTGAGGCGCCTCGGCCA |
| Tph2 gRNA #1 | CTGCAGCAGGGGGTACTAG |
| Tph2 gRNA #5 | TTGCTGGCTGCATGGATCC |

Insulin: Provided below are gRNA sequences for insertion of a marker in the insulin gene.

| | | |
|---|---|---|
| Guide #1 | 70 | GGGGCAGGAGGCGCATCCACAGG |
| Guide #2 | 67 | GGGCAGGAGGCGCATCCACAGGG |

### Example 3

### Generation of Gut Organoids From iPS Cells

Human iPS cells were differentiated into gut organoids as described in McCracken, K. W., Howell, J. C., Wells, J. M. & Spence, J. R. Generating human intestinal tissue from pluripotent stem cells in vitro. Nature protocols 6, 1920-1928 (2011) with some modifications. STEMdiff™ Definitive Endoderm Kit (Stemcell Technologies) was used instead of Activin A for differentiation towards definitive endoderm. Gut organoids were passaged every 2-3 weeks until 360 days; the morphology was assessed periodically using immunohistochemistry.

### Example 4

### Production of Insulin-Producing Cells in Gut Organoids Derived from CRSPR-Modified Cells

CRSPR mutagenesis was used to introduce fluorescent markers (indicated in parentheses) into the following genes: Neurogenin3 (GFP), Tph2 (cerulean), Foxo1 (mOrange), and insulin2 (GFP). In Table 2, summarized are the different lines that have been derived to help in this process.

Table 2

Ngn3-EGFP
Allows identification of Ngn3+ progenitor cells

Foxo1-mOrange
Allows monitoring of Foxo1 expression

Tph2-Cerulean
Tryptophan hydroxylase 2 synthesizes 5HT
Increases when Foxo1 is inhibited
Insulin-positive cells lose 5HT expression

### Insulin-GFP

Allows monitoring of conversion

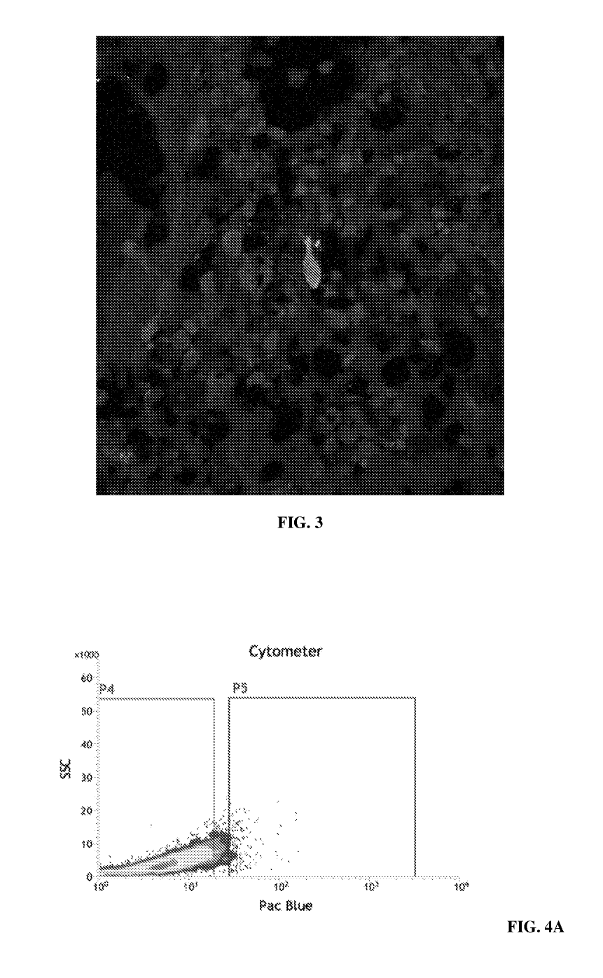

A first objective was to demonstrate that the CRSPR-modified cells can be differentiated into insulin-producing cells as expected. To this end, the Tph2 reporter cell line was differentiated into gut organoids (using the techniques described in Example 2 above), then the gut organoids were subjected to dominant-negative (DN) Foxo1 mutant to induce the formation of insulin-positive cells (FIG. 3, red).

Gut organoids derived from the Tph2 reporter cell line were transduced with adenovirus expressing a dominant-negative mutant FOXO1 (HA-Δ256) tagged with a hemagglutinin epitope to enhance detection (HA-Δ256), according to methods described in R. Bouchi, K. S. Foo, H. Hua, et al. FOXO1 inhibition yields functional insulin-producing cells in human gut organoid cultures, Nat Commun, 5 (2014), p. 4242; and Nakae, J., Kitamura, T., Silver, D. L. & Accili, D. The forkhead transcription factor Foxo1 (Fkhr) confers insulin sensitivity onto glucose-6-phosphatase expression. J. Clin. Invest. 108, 1359-1367 (2001). Insulin-producing cells were found, but no co-localization of GFP and insulin, indicating that 5HT expression is absent in insulin-producing cells. These results are consistent with previous work indicating that insulin expression is associated with loss of 5HT expression in 5HT-producing cells.

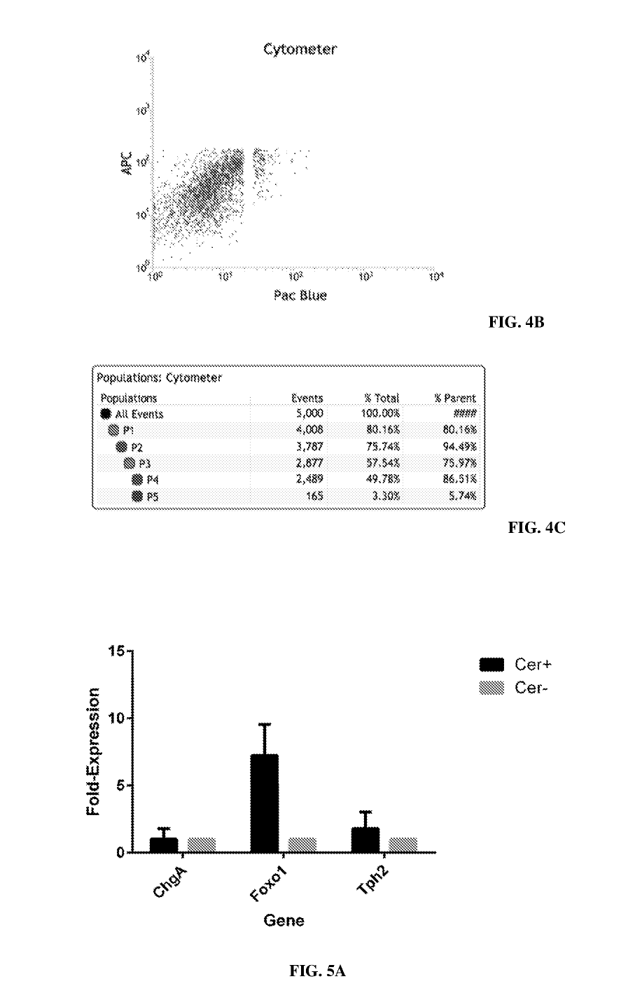

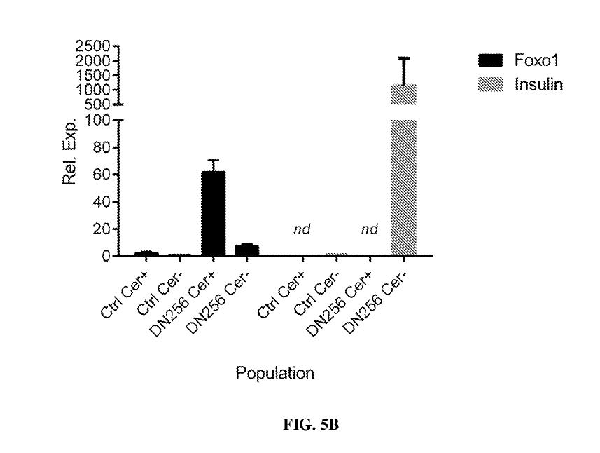

Next, fluorescence-activated cell sorting was used to isolate Tph2-GFP-expressing cells from the gut organoid cultures. As shown in FIG. 4, isolation of GFP-positive cells (P5 population) was successful, representing about 3% of all gutoid-derived cells, which is consistent with the frequency of 5HT-producing cells in the human intestine. These cells were then analyzed by qPCR. An enrichment in Foxo1 and Tph2 in the GFP+ population was detected (FIG. 5). While the enrichment in Tph2 is low, it is noted that the mRNA levels for this enzyme are low, and that it may not be the most abundant Tph isoform in the gut.

Next, the induction of insulin in response to transfection with the dominant negative Foxo1 was measured. As expected, Foxo1 could only be detected in cells transfected with the mutant construct. Please note that insulin induction occurred very strongly and only in cells that were no longer GFP-positive (indicated in the slide as Cer—FIG. 5). These important findings support the notion that induction of insulin is associated with suppression of the 5HT synthetic pathway. The data indicate that insulin and 5HT production are mutually exclusive, which confirms the original hypothesis that serotonin production diminishes as insulin production increases.

From the foregoing results, it is believed that the generated reporter cell lines faithfully recapitulate the 5HT-producing lineage in iPSC-derived gut organoids. Further, these cells are able to undergo differentiation and conversion into insulin-producing cells when Foxo1 is inhibited. The disappearance of Tph2 reporter activity following Foxo1 inhibition is consistent with the hypothesis that Foxo1 inhibition causes the conversion of intestinal 5HT-expressing cells into insulin-producing cells. The reporter cell lines described herein provide for the development of a screening tool to improve the efficiency of the conversion process and identify potential Foxo1-independent pathways to achieve the conversion in vivo through pharmacological means. It is important to note that the ability to isolate and characterize these cells by flow cytometry enables multiple uses of the reporter cells for different lines of research.

RNA isolation and RT-PCR. Standard Methods were used for RNA extraction and qRT-PCR (Invitrogen) as set forth in Talchai, C., Xuan, S., Kitamura, T., Depinho, R. A. & Accili, D. Generation of functional insulin-producing cells in the gut by Foxo1 ablation. Nat. Genet. 44, 406-412 (2012). Primer sequences are listed in Supplementary Table 2 of R. Bouchi, K. S. Foo, H. Hua, et al. FOXO1 inhibition yields functional insulin-producing cells in human gut organoid cultures, Nat Commun, 5 (2014), p. 4242.

Further details of the qPCR are provided below:
1. Using a standard mRNA isolation kit (we use the Qiagen RNeasy kits), follow instructions to isolate mRNA.
2. Using a standard reverse transcriptase kit (we use Quanta Biosciences' Script cDNA Supermix), generate cDNA from the isolated mRNA.
3. Dilute the cDNA 5× and use the following reaction components to prepare the qPCR reaction:
   For each well:
   a. 7.5 ul SYBR Green
   b. 2 ul total of primer (1 ul of each, 4 uM)
   c. 2 ul cDNA
   d. 3.5 ul H2O

Sorting of Single Cells from Gut Organoids: Gutoids grown in 4-well plates were washed once with PBS. Gutoids were then extracted from matrigel by trituration with a 1000 ul pipette and spun down at 250 g for 3 minutes in a 15 ml falcon tube. The PBS was aspirated and pre-warmed accutase was added at 500 ul/well of gutoids. The falcon tube was placed in a 37C water bath for 20 minutes, with trituration down every 5 minutes. 1× volume of basal media was added up to inactivate the accutase, and the mixture was pipetted 10×. The tube was then spun down again at 250G, the supernatant removed, and the cells resuspended in 2 mL of PBS for sorting. More details of this technique are provided below:
1. Pre-warm Accutase @37C.
2. Wash well with PBS 1× without disturbing matrigel mound.
3. Dissect gutoids from matrigel and cut into small pieces. Put into a low-binding 15 ml falcon with PBS.
4. Spin down (250 g).
5. Remove PBS and add warm Accutase (500 ul per well of gutoids).
6. Incubate at 37° C. for 20 mM. Pipette it vigorously every 5 mM with a low-binding 1,000-μl pipette for thorough dissociation.
7. Add basal media to inactivate Accutase. Triturate 10× with low-binding P1000 pipette tip.
8. Spin down (250 g).
9. Remove supernatant. Resuspend in 2 mL PBS.
10. Filter cells through blue-capped cell strainer into polypropylene sorting tube.
11. Add Sytox Red (Stock 5 uM) at 1:1000 dilution.
12. FACS sort by fluorescent protein reporter.

### Example 5

### Generation of Primary Gut Organoids

Duodenal biopsies from cadaveric donors were obtained directly from the OR. The mucosa was separated from surrounding connective tissue under a dissecting microscope with sterile fine scissors and forceps. The mucosa was cut into 5 mm pieces and kept on ice in DPBS. The pieces were then washed 10× in 10 ml of cold PBS. After removing the supernatant, the tissue was placed in 2.5 mM EDTA and rocked on a rocking shaker at 4° C. for 40 mM. Crypts were forcibly separated by 10× trituration, and spun down at 4° C. at 400 g for 3 min. The crypt pellet was then resuspended in matrigel and aliquoted onto a 24-well plate (50 ul/well). The matrigel mounds were hardened at 37C for 10 minutes, then growth media with Rho kinase inhibitor was added to each well.

Further Details of the Protocol are provided below, which are adapted from Fujii et al. Nature Protocols 2015 10:1474-1485

1: Keep the sample in 4° C. DPBS until processing. The sample can be preserved overnight at 4° C. in DPBS.

2: Before crypt isolation, thaw Matrigel on ice and keep it cold. Prewarm a 48-well plate in a 37° C. incubator. Add 5 ml of FBS to 45 ml of basal medium to prepare 10% (vol/vol) FBS medium.

3: For a surgically resected specimen, strip the underlying muscle layer off using fine scissors under a stereomicroscope, and then cut the sample into 5-mm pieces on a Petri dish. The dissected samples must be small enough to pass through the tip of a 10-ml pipette.

4: Place the dissected pieces of sample or biopsy specimens into a 15-ml centrifuge tube containing 10 ml of cold DPBS.

5: Wash the samples by pipetting with a 10-ml pipette at least ten times. For the subsequent steps, coat the inner surface of every 10-ml pipette with 10% (vol/vol) FBS medium before use to avoid adherence of the samples on the pipette wall.

6: Stand the tube still until the samples settle at the bottom. Aspirate the supernatant with a 10-ml pipette and add 10 ml of cold DPBS.

7: Repeat Steps 18 and 19 5-10 times until the supernatant is free of debris. Thorough washing of the sample is crucial to avoid bacterial contamination.

8: Add 10 ml of cold DPBS supplemented with 2.5 mM EDTA to the tube. Place the tube on a rocking shaker and rock it gently at 4° C. for 40 min

9: After treatment with EDTA, stand the tube still until the samples settle to the bottom of the tube, and then aspirate the supernatant.

10: Add 10 ml of cold DPBS and pipette up and down at least ten times with a 10-ml pipette. The crypts will be released into the supernatant by pipetting. Place the supernatant containing the isolated crypts into a new 15-ml tube.

11: Spin the crypts at 4° C. at 400 g for 3 min Remove the supernatant and place the tube on ice.

12: Suspend the pellet in 1 ml of DPBS. Drop 20 μl of the crypt suspension on a Petri dish. Count the number of crypts under a stereomicroscope and calculate the total number of crypts.

13: Add 9 ml of cold DPBS to the tube and spin the crypts at 4° C. at 400 g for 3 min Aspirate and discard the supernatant.

14: Suspend the crypts with Matrigel. Use a ratio of crypts to Matrigel that will allow 50-200 crypts in 25 μl of Matrigel.

15: Dispense 25 μl of the crypt-Matrigel suspension into the center of each well of a 48-well plate using a 200-μl pipette.

Place the plate in a 37° C. incubator for 10 min to solidify the Matrigel.

16: Add 250 μl of WENRAS medium supplemented with 10 μM Y-27632 to each well, and incubate the plate at 37° C.

### Example 6

### Histochemical Analysis of Primary Gut Organoids and Effects of Foxo1 Ablation in Primary Gut Organoids with a Dominant-Negative Construct

Primary Human Gut Organoids were produced as described in Example 5. The gut organoids were then subjected to the dominant negative construct (DN256) and processed for histochemical analysis.

Methods and Materials-Histochemical Analysis adapted from R. Bouchi, K. S. Foo, H. Hua, et al. FOXO1 inhibition yields functional insulin-producing cells in human gut organoid cultures, Nat Commun, 5 (2014), p. 4242

Generally, gut organoids were isolated from Matrigel, rinsed in phosphate-buffered saline and fixed in 4% phosphate-buffered paraformaldehyde for 15 min at room temperature. We fixed human gut specimens in the same buffer overnight. After fixation, organoids or gut specimens were incubated in 30% phosphate-buffered sucrose overnight at 4_C and embedded into Cryomold (Sakura Finetek) for subsequent frozen-block preparation. 6-mm-thick sections were cut from frozen blocks, and incubated with HistoVT One, using Blocking One (both from Nacalai USA) to block nonspecific binding8. Sections were incubated with primary antibodies for 12 h at 4_C, followed by incubation with secondary antibodies for 30 min at room temperature. Catalogue numbers and dilutions used for each antibody in Supplementary Table 1 for R. Bouchi, et al. Nat Commun, 5 (2014), p. 4242. Alexaconjugated donkey and goat secondary antibodies (Molecular Probes) were used. After the final wash, cells were viewed using a confocal microscopy (Zeiss LSM 710). Cells were counterstained DNA with 40,6-diamidino-2-phenylindole (DAPI, Cell Signaling).

More detailed protocols for processing of the tissue and immunohistochemical staining is provided below:

### For Parrafin Sections:

### I: Deparaffinization/Rehydration

Note: Place slides in containers for 5 minutes each. Each container holds 100 mL of solution. Can refer to R&D IHC/ICC protocols online for reference. Solutions 5-9 should be made fresh each time. The others can be topped off. (IF FROZEN: SKIP, THIS PART IS NOT REQUIRED, MOVE ONTO ANTIGEN UNMASKING).

1. Xylene

2. Xylene

3. 100% EtOH

4. 100% EtOH

5. 90% EtOH

6. 70% EtOH

7. 50% EtOH

8. Distilled H2O

9. PBS

For Frozen Sections: Air-dry the sections at room temperature, or at 55C, for 20 minutes.
Then, proceed with antigen unmasking, similar to paraffin-embedded sections unless otherwise noted:
II: Antigen Unmasking for Paraffin-embedded sections
1. Make up 20 mL of 1× HistoVT One (dilute 2 mL of the 10× stock in 18 mL deionized H2O) in the small slide container
2. Heat up H2O in glass container (water bath) to 90C (70C for frozen sections) using thermometer and plate heater.
3. Place small slide box container in the water bath for 20 min, wrapping it securely with parafilm.
4. Wash 1× with PBST, making sure to NOT let the slides dry.
   III: Blocking with One Histo
1. Get the One Histo bottle from 4C
2. Take out 1 slide at a time, tapping off excess water and drying around sample with a kimwipe.
3. Draw a circle around the sample with hydrophobic pap pen.
4. Add enough One Histo to cover sample (1-2 drops)
5. Incubate @RT in black slidebox for 1 hr.

### IV: Primary Antibody

1. Dilute 50 ul blocking One Histo in 950 ul PBS with 0.1% Tween20. This will be the diluent for the primary antibody.
2. Prepare ˜100 uL of primary antibody diluted in the OneHisto+PBST mixture per section(1. Insulin-Guinea pig, DAKO; c-peptide, mouse, Millipore;.
3. Add 50-100 ul of primary antibody to each section. Make sure the hydrophobic perimeter is still intact before adding. Add excess antibody mixture to ensure O/N evaporation will not dry out the tissue.
4. Incubate in coldroom in black slidebox O/N.

### V: Secondary Antibody

1. Wash in 1× PBS 0.05% Tween20 for 10 minutes, total 3×.
2. Dilute secondary Ab in 1× PBS 0.05% Tween20 (1:500).
3. Incubate with secondary Ab in black slidebox at RT for 30 minutes to 1 hr.
4. Wash with 1× PBS 0.05% Tween20 again for 10 minutes, total 2×
5. Wash 10 minutes in 1× PBS.

VI: Hoechst (LH-side, in large white cylinder. Stock is 10 mg/ml)
1. Dilute to 3-5 ug/ml in PBS (usually 30 ul in 100 mL of PBS to fill an entire slide box)
2. Incubate for 15 min @RT on shaker.
3. Wash 2× PBST and 1× PBS.

### VII: Mounting

1. Dry off slide with kimwipe and add 1 drop of mounting solution (Prolong Gold antifade reagent w/DAPI or Vectashield)
2. Place coverslip on top, letting one side fall first to minimize bubbles. Slowly lower the coverslip. DO NOT move coverslip after letting it fall, as it will distort the sample.
3. Seal the outer edges with clear nail polish.
4. Place in slide folder at 4C for short-term storage, −20C for long-term.
5. The mounted slides are then imaged with confocal imaging (Zeiss LSM 710).

Adenoviral transfection: Ad-CMV-FOXO1-D256 expressing a mutant version of FOXO1 containing its amino domain (corresponding to amino-acid residues 1-256) has been described_Nakae J et al, J. Clin. Invest. 2001, 108(9):1359-67. Briefly, overlap extension PCR was used to generate the Δ256 mutant FoxO1 construct. Sequence accession # GenBank: AF126056.1. The 5′ fragment contained a unique BglII restriction site at the 5′ end, and a mutagenic oligonucleotide at the 3′ end; the 3′ fragment contained a unique Agel restriction site at the 3′ end, and the mutagenic oligonucleotide at the 5′ end. Following amplification of each individual fragment, a second PCR was carried out to generate a single fragment containing the mutation and straddling the two unique restriction sites at the 5′ and 3′ ends, respectively. The resulting PCR fragment was used to replace the wild-type sequence in a pCMV5-cMyc expression vector. To generate the Δ256 mutant, the following primers were employed; 1, 5′-GACCTCATCACCAAGGCCATC-3′, corresponding to nucleotides 490-510; 2, 5′-GGCCCATCATTACATTTTGGCCCAGGAC-3′, corresponding to nucleotides 1489-1462; primer 3, 5′-TTTACTGTTCTAGTCCATGGA-3′, corresponding to nucleotides 777-757; primer 4, 5′-TCCATGGACTAGAACAGTAAA-3′, corresponding to nucleotides 757-777. After digestion with KpnI and XbaI, the PCR fragment was subcloned into KpnI- and XbaI-treated pCMV5/c-Myc. DNA encoding the HA-tagged mutant Foxo1 was subcloned into pAxCAwt, and adenovirus vectors containing these cDNAs were generated by transfecting HEK 293 cells with the corresponding pAxCAwt plasmid, together with a DNA-terminal protein complex,

Adenoviruses were prepared for transfection by CsCl density centrifugation to a titre of 2.5_10¹² viral particles m1⁻¹ (1.6_10¹¹ plaque-forming units ml⁻¹) for Ad-CMV-FOX01- D256 and 2.4_10¹² vp ml⁻¹ (1.9_10¹¹ p.f.u. ml⁻¹) for the Gfp control. Gutorganoids were mechanically dissociated from Matrigel, cut in half and incubated in DMEM/F12 containing 10 mM ROCK inhibitor (Y27632) with 1 ml of adenovirus solution for 3 h at 37° C. in a 5% CO₂ incubator and then washed with phosphate buffered saline three times. After transduction, mini-guts were embedded into fresh Matrigel again and incubated with intestinal growth medium as described in McCracken, K. W., Howell, J. C., Wells, J. M. & Spence, J. R. Generating human intestinal tissue from pluripotent stem cells in vitro. Nature protocols 6, 1920-1928 (2011).

Virus Infection of Gutoids:
1) Choose 3-4 gutoids and remove from matrigel
2) Cut in half and incubate in DMEM/F12 containing 10 mM Rock inhibitor (Y27632) with 1 ml of adenovirus solution for 3h at 37C (in 4-well plates). The virus can be diluted 1:2000 or 1:10000.
3) Wash 3× with PBS
4) Embed back in fresh matrigel with intestine media.
5) Culture for an additional 7 days, changing the media every 3 days.

RNA isolation and RT-PCR. Standard Methods were used for RNA extraction and qRT-PCR (Invitrogen) as set forth in Talchai, C., Xuan, S., Kitamura, T., Depinho, R. A. & Accili, D. Generation of functional insulin-producing cells in the gut by Foxo1 ablation. Nat. Genet. 44, 406-412 (2012). Primer sequences are listed in Supplementary Table 2 of R. Bouchi, K. S. Foo, H. Hua, et al. FOXO1 inhibition yields functional insulin-producing cells in human gut organoid cultures, Nat Commun. 5 (2014), p. 4242

### Results for Example 6

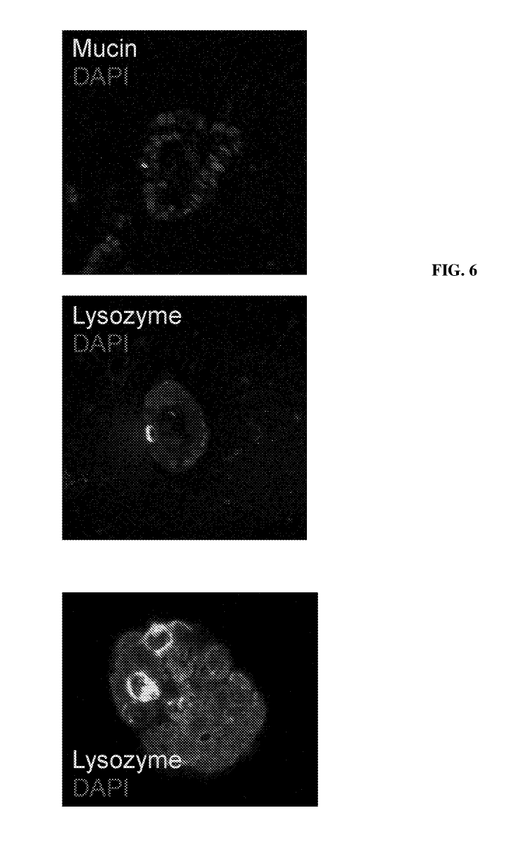

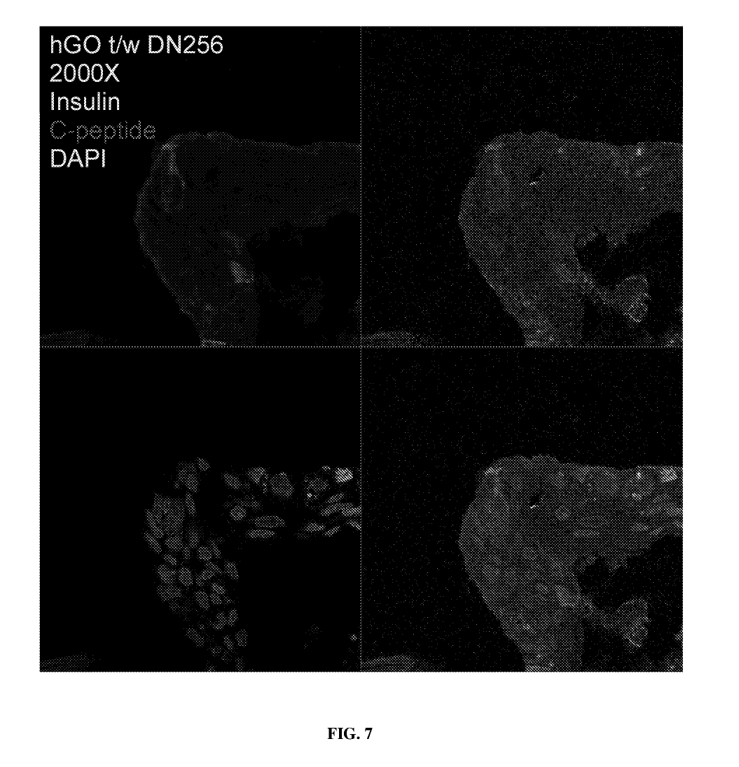

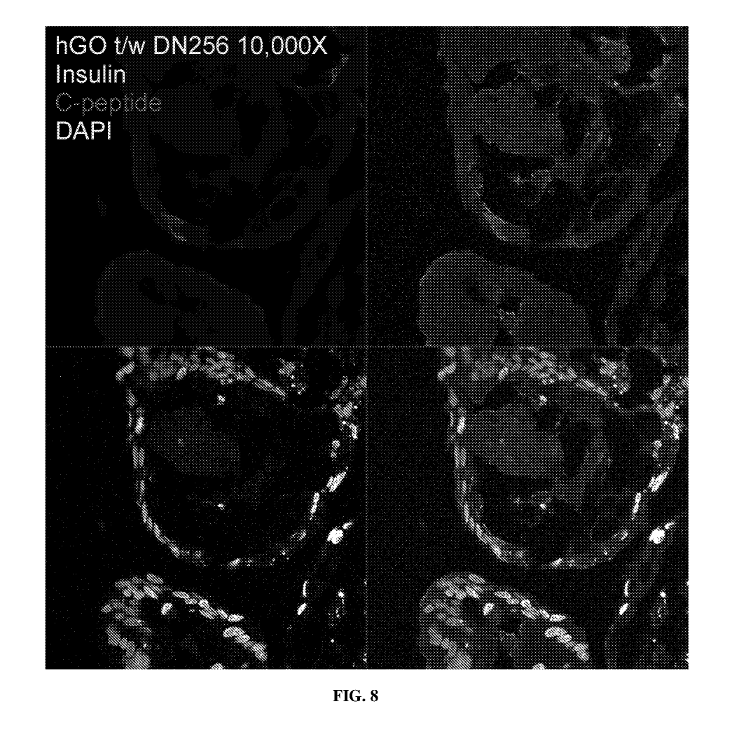

FIG. 6 represents a series of images showing that the organoids contain the relevant cell types: Mucin, Lysozyme (green). The lower right slide is a merge of the other three slides. The effect of direct Foxo inhibition through a dominant-negative construct DN256 was examined FIG. 7 relates to histochemical analysis of slides of primary human gut organoids that were treated with the dominant negative construct (DN256). As can be seen, treatment of the organoids with the DN256 construct led to production of insulin producing cells, represented by the green cells. It was found that there was some non-specific binding to the same antibody as a control, which was believed to be caused by toxicity of the adenovirus.

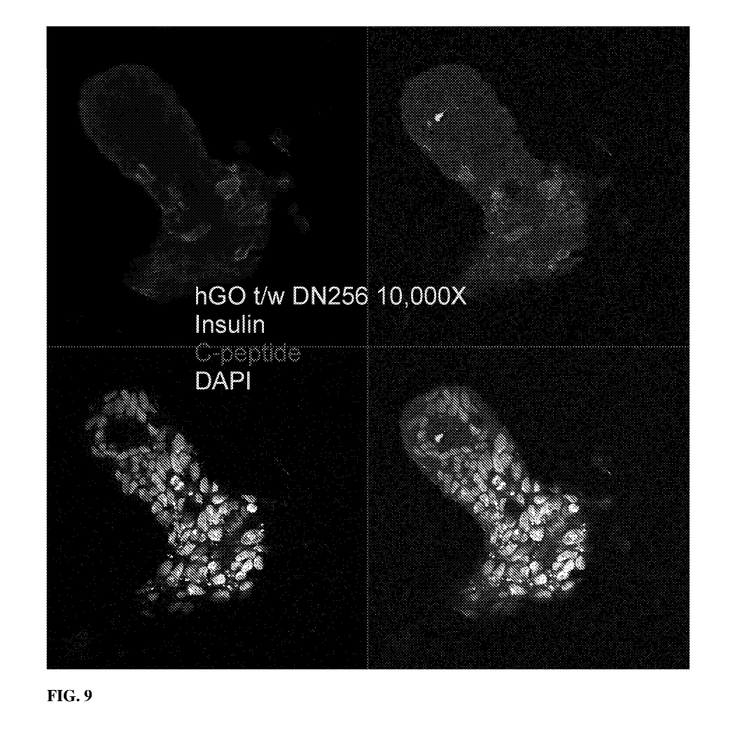

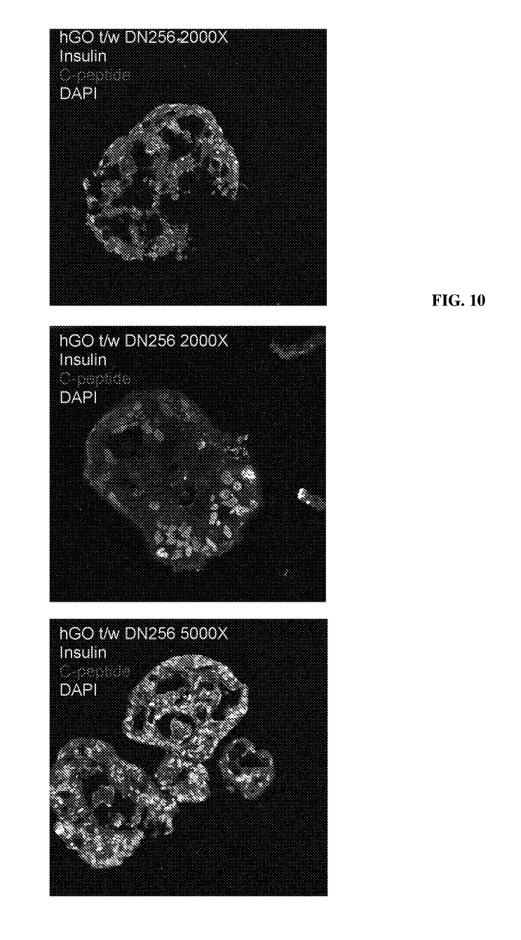

FIGS. 8 and 9 represent histochemical analysis of organoids using a much lower concentration of the DN256 (1:10,000) to avoid cell toxicity due to the adenovirus. At this dilution, the virus still had the ability to generate insulin-producing cells (green), and the organoids showed fewer signs of cell death (fragmented nuclei in white). FIG. 10 shows dose-response experiments in which higher adenovirus concentrations were used (1:2,000; 1:5,000), with non-specific effects on cell survival (fragmented nuclei, white). Non-specific staining can be observed as a low-level green (insulin) or blue (C-peptide) background which is often due to the stickiness of dead cell debris.

FIG. 11 shows data from RNA analysis of the converted primary organoids treated with DN256. 2000×, 5000×, and 10000× denote dilution of the virus. Ryo-insulin indicates the qPCR primer used. The data of FIG. 11 shows that blocking Foxo1 with DN256 resulted in induction of Insulin and Neurogenin, as expected. The Y-axis represents “relative expression” of the gene. This is a standardized metric for expression levels once the necessary controls have been accounted for. Tph2 is high because there is a compensatory induction of Tph2 expression whenever cells are treated with FoxO DN256. This suggests that cells which may be converting to insulin+ cells may have previously been serotonin producing cells. As the cells lose serotonin production, regulatory mechanisms attempt to compensate by increasing Tph2 expression (an enzyme that makes serotonin).

### Example 7

### Production of Cell Monolayers Gut Progenitor and Enteroendocrine Cells

To simplify the handling of gut organoid cultures, methods have been established to grow gut stem cells in monolayers. This approach is based on a simplified modification of the existing method to generate gut organoid cultures described by the Karp laboratory (Yin X, Farin H F, van Es J H, Clevers H, Langer R, and Karp J M, Niche-independent high-purity cultures of Lgr5+ intestinal stem cells and their progeny. Nature methods. 2014;11(1):106-12.) Briefly, iPS cells were cultured in STEMdiff medium from Stemcell Technologies to differentiate cells into definitive endoderm. Once the endoderm begins to bud out of the monolayer, it is mechanically removed and placed in EDTA to generate a single cell suspension. The cell suspension is re-plated on collagen-coated dishes and treated sequentially with the Gsk3 inhibitor CHIR (3 μM, Stemgent) and valproic acid (1 mM, Sigma-Aldrich). This population should be enriched in LGR5 stem cells. To assess this point, cells passaged and their cellular composition is analyzed by qPCR and immunohistochemistry. Increased levels of Lgr5 were found, as well as increased markers of early gut cell progenitor cell types, including BMI, EphrR, and NGN3. Immunohistochemical analysis is more challenging, owing to the dearth of antibodies that react with gut stem cells. However, it has been shown that the cultures are enriched in progenitor cell markers, Sox9, Oct4, and L-Myc. These data demonstrate the ability to generate monolayer cell cultures that can replace the gut organoid system in a screening assay. It has also been shown that these cultures can last for up to two weeks, which should be a sufficiently broad timeframe to attempt to generate endocrine progenitors and to knock down FOXO1 for the purpose of generating insulin-producing cells.

In addition, the genetically modified cells harboring fluorescent reporter genes fused to Ngn3, Foxo1, Thp or insulin, or combination thereof described in Example 2 herein, are subjected to the differentiation protocol described above. The resultant cells may be flow-sorted based on fluorescence of one or more of these target genes. Monolayer or gut organoid cultures of these genetically modified cells provides for a robust screening platform and differentiation monitoring tool to elucidate cellular mechanisms involved in the conversion of gut cells into insulin producing cells, as well as the ability to screen for agents that induce the production of insulin+ cells in the gut.

The invention is illustrated herein by the experiments described by the following examples, which should not be construed as limiting. The contents of all references, pending patent applications and published patents, cited throughout this application are hereby expressly incorporated by reference. Those skilled in the art will understand that this invention may be embodied in many different forms and should not be construed as limited to the embodiments set forth herein. Rather, these embodiments are provided so that this disclosure will fully convey the invention to those skilled in the art. Many modifications and other embodiments of the invention will come to mind in one skilled in the art to which this invention pertains.

### Gene and MRNA Sequences:

| All are human sequences. | |
|---|---|
| HUMAN INSULIN Ref Gene Sequence (GenBank Accession No. NG_007114, | |
| (SEQ ID NO. 5)) | |
| mRNA | |
| ORIGIN | |
| 1 | agccctccag gacaggctgc atcagaagag gccatcaagc agatcactgt ccttctgcca |
| 61 | tggccctgtg gatgcgcctc ctgcccctgc tggcgctgct ggccctctgg ggacctgacc |
| 121 | cagccgcagc ctttgtgaac caacacctgt gcggctcaca cctggtggaa gctctctacc |
| 181 | tagtgtgcgg ggaacgaggc ttcttctaca cacccaagac ccgccgggag gcagaggacc |
| 241 | tgcaggtggg gcaggtggag ctgggcgggg gccctggtgc aggcagcctg cagcccttgg |
| 301 | ccctggaggg gtccctgcag aagcgtggca ttgtggaaca atgctgtacc agcatctgct |
| 361 | ccctctacca gctggagaac tactgcaact agacgcagcc cgcaggcagc cccacacccg |
| 421 | ccgcctcctg caccgagaga gatggaataa agcccttgaa ccagcaaaa |
| HUMAN INSULIN Protein | |
| Origin | |
| 1 | MALWMRLLPL LALLALWGPD PAAAFVNQHL CGSHLVEALY LVCGERGFFY TPKTRREAED |
| 61 | LQVGQVELGG GPAGGGLQPL ALEGSLQKRG IVEQCCTSIC SLYQLENYCN |
| HUMAN FOXO1 | |
| GENE SEQ Genbank (Accession No. NG_023244, SEQ ID NO. 4) | |
| MRNA SEQ | |
| 1 | gcagccgcca cattcaacag gcagcagcgc agcgggcgcg ccgctgggga gagcaagcgg |
| 61 | cccgcggcgt ccgtccgtcc ttccgtccgc ggccctgtca gctggagcgc ggcgcaggct |
| 121 | ctgccccggc ccggcggctc tggccggccg tccagtccgt gcggcggacc ccgaggagcc |
| 181 | tcgatgtgga tggccccgcg aagttaagtt ctgggctcgc gcttccactc cgccgcgcct |
| 241 | tcctcccagt ttccgtccgc tcgccgcacc ggcttcgttc ccccaaatct cggaccgtcc |
| 301 | cttcgcgccc cctccccgtc cgcccccagt gctgcgttct ccccctcttg gctctcctgc |
| 361 | ggctggggga ggggcggggg tcaccatggc cgaggcgcct caggtggtgg agatcgaccc |
| 421 | ggacttcgag ccgctgcccc ggccgcgctc gtgcacctgg ccgctgccca ggccggagtt |
| 481 | tagccagtcc aactcggcca cctccagccc ggcgccgtcg ggcagcgcgg ctgccaaccc |
| 541 | cgacgccgcg gcgggcctgc cctcggcctc ggctgccgct gtcagcgccg acttcatgag |
| 601 | caacctgagc ttgctggagg agagcgagga cttcccgcag gcgcccggct ccgtggcggc |
| 661 | ggcggtggcg gcggcggccg ccgcggccgc caccgggggg ctgtgcgggg acttccaggg |
| 721 | cccggaggcg ggctgcctgc acccagcgcc accgcagccc ccgccgcccg ggccgctgtc |
| 781 | gcagcacccg ccggtgcccc ccgccgccgc tgggccgctc gcggggcagc cgcgcaagag |
| 841 | cagctcgtcc cgccgcaacg cgtggggcaa cctgtcctac gccgacctca tcaccaaggc |
| 901 | catcgagagc tcggcggaga agcggctcac gctgtcgcag atctacgagt ggatggtcaa |
| 961 | gagcgtgccc tacttcaagg ataagggtga cagcaacagc tcggcgggct ggaagaattc |
| 1021 | aattcgtcat aatctgtccc tacacagcaa gttcattcgt gtgcagaatg aaggaactgg |
| 1081 | aaaaagttct tggtggatgc tcaatccaga gggtggcaag agcgggaaat ctcctaggag |
| 1141 | aagagctgca tccatggaca acaacagtaa atttgctaag agccgaagcc gagctgccaa |
| 1201 | gaagaaagca tctctccagt ctggccagga gggtgctggg gacagccctg gatcacagtt |
| 1261 | ttccaaatgg cctgcaagcc ctggctctca cagcaatgat gactttgata actggagtac |
| 1321 | atttcgccct cgaactagct caaatgctag tactattagt gggagactct cacccattat |
| 1381 | gaccgaacag gatgatcttg gagaagggga tgtgcattct atggtgtacc cgccatctgc |
| 1441 | cgcaaagatg gcctctactt tacccagtct gtctgagata agcaatcccg aaaacatgga |
| 1501 | aaatcttttg gataatctca accttctctc atcaccaaca tcattaactg tttcgaccca |
| 1561 | gtcctcacct ggcaccatga tgcagcagac gccgtgctac tcgtttgcgc caccaaacac |
| 1621 | cagtttgaat tcacccagcc caaactacca aaaatataca tatggccaat ccagcatgag |
| 1681 | ccctttgccc cagatgccta tacaaacact tcaggacaat aagtcgagtt atggaggtat |
| 1741 | gagtcagtat aactgtgcgc ctggactctt gaaggagttg ctgacttctg actctcctcc |
| 1801 | ccataatgac attatgacac cagttgatcc tggggtagcc cagcccaaca gccgggttct |
| 1861 | gggccagaac gtcatgatgg gccctaattc ggtcatgtca acctatggca gccaggcatc |
| 1921 | tcataacaaa atgatgaatc ccagctccca tacccaccct ggacatgctc agcagacatc |
| 1981 | tgcagttaac gggcgtcccc tgccccacac ggtaagcacc atgccccaca cctcgggtat |
| 2041 | gaaccgcctg acccaagtga agacacctgt acaagtgcct ctgccccacc ccatgcagat |
| 2101 | gagtgccctg gggggctact cctccgtgag cagctgcaat ggctatggca gaatgggcct |
| 2161 | tctccaccag gagaagctcc caagtgactt ggatggcatg ttcattgagc gcttagactg |
| 2221 | tgacatggaa tccatcattc ggaatgacct catggatgga gatacattgg attttaactt |
| 2281 | tgacaatgtg ttgcccaacc aaagcttccc acacagtgtc aagacaacga cacatagctg |
| 2341 | ggtgtcaggc tgagggttag tgagcaggtt acacttaaaa gtacttcaga ttgtctgaca |
| 2401 | gcaggaactg agagaagcag tccaaagatg tctttcacca actccctttt agttttcttg |
| 2461 | gttaaaaaaa aaaacaaaaa aaaaaaccct ccttttttcc tttcgtcaga cttggcagca |
| 2521 | aagacatttt tcctgtacag gatgtttgcc caatgtgtgc aggttatgtg ctgctgtaga |
| 2581 | taaggactgt gccattggaa atttcattac aatgaagtgc caaactcact acaccatata |
| 2641 | attgcagaaa agattttcag atcctggtgt gctttcaagt tttgtatata agcagtagat |
| 2701 | acagattgta tttgtgtgtg tttttggttt ttctaaatat ccaattggtc caaggaaagt |
| 2761 | ttatactctt tttgtaatac tgtgatgggc ctcatgtctt gataagttaa acttttgttt |
| 2821 | gtactacctg ttttctgcgg aactgacgga tcacaaagaa ctgaatctcc attctgcatc |
| 2881 | tccattgaac agccttggac ctgttcacgt tgccacagaa ttcacatgag aaccaagtag |
| 2941 | cctgttatca atctgctaaa ttaatggact tgttaaactt ttggaaaaaa aaagattaaa |
| 3001 | tgccagcttt gtacaggtct tttctatttt tttttgttta ttttgttatt tgcaaatttg |
| 3061 | tacaaacatt taaatggttc taatttccag ataaatgatt tttgatgtta ttgttgggac |
| 3121 | ttaagaacat ttttggaata gatattgaac tgtaataatg ttttcttaaa actagagtct |
| 3181 | actttgttac atagtcagct tgtaaatttt gtggaaccac aggtatttgg ggcagcattc |
| 3241 | ataattttca ttttgtattc taactggatt agtactaatt ttatacatgc ttaactggtt |
| 3301 | tgtacacttt gggatgctac ttagtgatgt ttctgactaa tcttaaatca ttgtaattag |
| 3361 | tacttgcata ttcaacgttt caggccctgg ttgggcagga aagtgatgta tagttatgga |
| 3421 | cactttgcgt ttcttattta ggataactta atatgttttt atgtatgtat tttaaagaaa |
| 3481 | tttcatctgc ttctactgaa ctatgcgtac tgcatagcat caagtcttct ctagagacct |
| 3541 | ctgtagtcct gggaggcctc ataatgtttg tagatcagaa aagggagatc tgcatctaaa |
| 3601 | gcaatggtcc tttgtcaaac gagggatttt gatccacttc accattttga gttgagcttt |
| 3661 | agcaaaagtt tcccctcata attctttgct cttgtttcag tccaggtgga ggttggtttt |
| 3721 | gtagttctgc cttgaggaat tatgtcaaca ctcatacttc atctcattct cccttctgcc |
| 3781 | ctgcagatta gattacttag cacactgtgg aagtttaagt ggaaggaggg aatttaaaaa |
| 3841 | tgggacttga gtggtttgta gaatttgtgt tcataagttc agatgggtag caaatggaat |
| 3901 | agaacttact taaaaattgg ggagatttat ttgaaaacca gctgtaagtt gtgcattgag |
| 3961 | attatgttaa aagccttggc ttaagaattt gaaaatttct ttagcctgta gcaacctaaa |
| 4021 | ctgtaattcc tatcattatg ttttattact ttccaattac ctgtaactga cagaccaaat |
| 4081 | taattggctt tgtgtcctat ttagtccatc agtattttca agtcatgtgg aaagcccaaa |
| 4141 | gtcatcacaa tgaagagaac aggtgcacag cactgttcct cttgtgttct tgagaaggat |
| 4201 | ctaatttttc tgtatatagc ccacatcaca cttgctttgt cttgtatgtt aattgcatct |
| 4261 | tcattggctt ggtatttcct aaatgtttaa caagaacaca agtgttcctg ataagatttc |
| 4321 | ctacagtaag ccagctctat tgtaagcttc ccactgtgat gatcattttt ttgaagattc |
| 4381 | attgaacagc caccactcta tcatcctcat tttggggcag tccaagacat agctggtttt |
| 4441 | agaaacccaa gttcctctaa gcacagcctc ccgggtatgt aactgaactt ggtgccaaag |
| 4501 | tacttgtgta ctaatttcta ttactacgta ctgtcacttt cctcccgtgc cattactgca |
| 4561 | tcataataca aggaacctca gagcccccat ttgttcatta aagaggcaac tacagccaaa |
| 4621 | atcactgtta aaatcttact acttcatgga gtagctctta ggaaaatata tcttcctcct |
| 4681 | gagtctgggt aattatacct ctcccaagcc cccattgtgt gttgaaatcc tgtcatgaat |
| 4741 | ccttggtagc tctctgagaa cagtgaagtc cagggaaagg catctggtct gtctggaaag |
| 4801 | caaacattat gtggcctctg gtagtttttt tcctgtaaga atactgactt tctggagtaa |
| 4861 | tgagtatata tcagttattg tacatgattg ctttgtgaaa tgtgcaaatg atatcaccta |
| 4921 | tgcagccttg tttgatttat tttctctggt ttgtactgtt attaaaagca tattgtatta |
| 4981 | tagagctatt cagatatttt aaatataaag atgtattgtt tccgtaatat agacgtatgg |
| 5041 | aatatattta ggtaatagat gtattacttg gaaagttctg ctttgacaaa ctgacaaagt |
| 5101 | ctaaatgagc acatgtatcc cagtgagcag taaatcaatg gaacatccca agaagaggat |
| 5161 | aaggatgctt aaaatggaaa tcattctcca acgatataca aattggactt gttcaactgc |
| 5221 | tggatatatg ctaccaataa ccccagcccc aacttaaaat tcttacattc aagctcctaa |
| 5281 | gagttcttaa tttataacta attttaaaag agaagtttct tttctggttt tagtttggga |
| 5341 | ataatcattc attaaaaaaa atgtattgtg gtttatgcga acagaccaac ctggcattac |
| 5401 | agttggcctc tccttgaggt gggcacagcc tggcagtgtg gccaggggtg gccatgtaag |
| 5461 | tcccatcagg acgtagtcat gcctcctgca tttcgctacc cgagtttagt aacagtgcag |
| 5521 | attccacgtt cttgttccga tactctgaga agtgcctgat gttgatgtac ttacagacac |
| 5581 | aagaacaatc tttgctataa ttgtataaag ccataaatgt acataaatta tgtttaaatg |
| 5641 | gcttggtgtc tttcttttct aattatgcag aataagctct ttattaggaa ttttttgtga |
| 5701 | agctattaaa tacttgagtt aagtcttgtc agccacaa |
| Foxo1 Protein Seq | |
| 1 | maeapqvvei dpdfeplprp rsctwplprp efsqsnsats spapsgsaaa npdaaaglps |
| 61 | asaaavsadf msnlsllees edfpqapgsv aaavaaaaaa aatgglcgdf qgpeagclhp |
| 121 | appqppppgp lsqhppvppa aagplagqpr kssssrrnaw gnlsyadlit kaiessaekr |
| 181 | ltlsqiyewm vksvpyfkdk gdsnssagwk nsirhnlslh skfirvqneg tgksswwmln |
| 241 | peggksgksp rrraasmdnn skfaksrsra akkkaslqsg qegagdspgs qfskwpaspg |
| 301 | shsnddfdnw stfrprtssn astisgrlsp imteqddlge gdvhsmvypp saakmastlp |
| 361 | slseisnpen menlldnlnl lssptsltvs tqsspgtmmq qtpcysfapp ntslnspspn |
| 421 | yqkytygqss msplpqmpiq tlqdnkssyg gmsqyncapg llkelltsds pphndimtpv |
| 481 | dpgvaqpnsr vlgqnvmmgp nsvmstygsq ashnkmmnps shthpghaqq tsavngrplp |
| 541 | htvstmphts gmnrltqvkt pvqvplphpm qmsalggyss vsscngygrm gllhqeklps |
| 601 | dldgmfierl dcdmesiirn dlmdgdtldf nfdnvlpnqs fphsvkttth swvsg |
| Human TPH1 Ref. Gen Seq (GeneBank Accession No. NG_011947 (SEQ ID NO. 3) | |
| mRNA Seq | |
| 1 | ttttagagaa ttactccaaa ttcatcatga ttgaagacaa taaggagaac aaagaccatt |
| 61 | ccttagaaag gggaagagca agtctcattt tttccttaaa gaatgaagtt ggaggactta |
| 121 | taaaagccct gaaaatcttt caggagaagc atgtgaatct gttacatatc gagtcccgaa |
| 181 | aatcaaaaag aagaaactca gaatttgaga tttttgttga ctgtgacatc aacagagaac |
| 241 | aattgaatga tatttttcat ctgctgaagt ctcataccaa tgttctctct gtgaatctac |
| 301 | cagataattt tactttgaag gaagatggta tggaaactgt tccttggttt ccaaagaaga |
| 361 | tttctgacct ggaccattgt gccaacagag ttctgatgta tggatctgaa ctagatgcag |
| 421 | accatcctgg cttcaaagac aatgtctacc gtaaacgtcg aaagtatttt gcggacttgg |
| 481 | ctatgaacta taaacatgga gaccccattc caaaggttga attcactgaa gaggagatta |
| 541 | agacctgggg aaccgtattc caagagctca acaaactcta cccaacccat gcttgcagag |
| 601 | agtatctcaa aaacttacct ttgctttcta aatattgtgg atatcgggag gataatatcc |
| 661 | cacaattgga agatgtctcc aactttttaa aagagcgtac aggtttttcc atccgtcctg |
| 721 | tggctggtta cttatcacca agagatttct tatcaggttt agcctttcga gtttttcact |
| 781 | gcactcaata tgtgagacac agttcagatc ccttctatac cccagagcca gatacctgcc |
| 841 | atgaactctt aggtcatgtc ccgcttttgg ctgaacctag ttttgcccaa ttctcccaag |
| 901 | aaattggctt ggcttctctt ggcgcttcag aggaggctgt tcaaaaactg gcaacgtgct |
| 961 | actttttcac tgtggagttt ggtctatgta aacaagatgg acagctaaga gtctttggtg |
| 1021 | ctggcttact ttcttctatc agtgaactca aacatgcact ttctggacat gccaaagtaa |
| 1081 | agccctttga tcccaagatt acctgcaaac aggaatgtct tatcacaact tttcaagatg |
| 1141 | tctactttgt atctgaaagt tttgaagatg caaaggagaa gatgagagaa tttaccaaaa |
| 1201 | caattaagcg tccatttgga gtgaagtata atccatatac acggagtatt cagatcctga |
| 1261 | aagacaccaa gagcataacc agtgccatga atgagctgca gcatgatctc gatgttgtca |
| 1321 | gtgatgccct tgctaaggtc agcaggaagc cgagtatcta acagtagcca gtcatccagg |
| 1381 | aacatttgag catcaattcg gaggtctggg ccatctcttg ctttccttga acacctgatc |
| 1441 | ctggagggac agcatcttct ggccaaacaa tattatcgaa ttccactact taaggaatca |
| 1501 | ctagtctttg aaaatttgta cctggatatt ctatttacca cttatttttt tgtttagttt |
| 1561 | tatttctttt tttttttggt agcagcttta atgagacaat ttatatacca tacaagccac |
| 1621 | tgaccaccca tttttaatag agaagttgtt tgacccaata gatagatcta atctcagcct |
| 1681 | aactctattt tccccaatcc tccttgagta aaatgaccct ttaggatcgc ttagaataac |
| 1741 | ttgaggagta ttatggcgct gactcatatt gttacctaag atccccttat ttctaaagta |
| 1801 | tctgttactt attgc |
| TPH1 Protein Seq. | |
| MIEDNKENKDHSLERGRASLIFSLKNEVGGLIKALKIFQEKHVNLLHIESRKSKRRNSEFEIFVDCDINRE | |
| QLNDIFHLLKSHTNVLSVNLPDNFTLKEDGMETVPWFPKKISDLDHCANRVLMYGSELDADHPGFKDNVYR | |
| KRRKYFADLAMNYKHGDPIPKVEFTEEEIKTWGTVFQELNKLYPTHACREYLKNLPLLSKYCGYREDNIPQ | |
| LEDVSNFLKERTGFSIRPVAGYLSPRDFLSGLAFRVFHCTQYVRHSSDPFYTPEPDTCHELLGHVPLLAEP | |
| SFAQFSQEIGLASLGASEEAVQKLATCYFFTVEFGLCKQDGQLRVFGAGLLSSISELKHALSGHAKVKPFD | |
| PKITCKQECLITTFQDVYFVSESFEDAKEKMREFTKTIKRPFGVKYNPYTRSIQILKDTKSITSAMNELQH | |
| DLDVVSDALAKVSRKPSI | |
| HUMAN TPH2 Ref Gene Seq (Genbank Accession No. NG_008279 (SEQ ID NO. 2)) | |
| MRNA SEQ | |
| 1 | cattgctctt cagcaccagg gttctggaca gcgccccaag caggcagctg atcgcacgcc |
| 61 | ccttcctctc aatctccgcc agcgctgcta ctgcccctct agtaccccct gctgcagaga |
| 121 | aagaatatta caccgggatc catgcagcca gcaatgatga tgttttccag taaatactgg |
| 181 | gcacggagag ggttttccct ggattcagca gtgcccgaag agcatcagct acttggcagc |
| 241 | tcaacactaa ataaacctaa ctctggcaaa aatgacgaca aaggcaacaa gggaagcagc |
| 301 | aaacgtgaag ctgctaccga aagtggcaag acagcagttg ttttctcctt gaagaatgaa |
| 361 | gttggtggat tggtaaaagc actgaggctc tttcaggaaa aacgtgtcaa catggttcat |
| 421 | attgaatcca ggaaatctcg gcgaagaagt tctgaggttg aaatctttgt ggactgtgag |
| 481 | tgtgggaaaa cagaattcaa tgagctcatt cagttgctga aatttcaaac cactattgtg |
| 541 | acgctgaatc ctccagagaa catttggaca gaggaagaag agctagagga tgtgccctgg |
| 601 | ttccctcgga agatctctga gttagacaaa tgctctcaca gagttctcat gtatggttct |
| 661 | gagcttgatg ctgaccaccc aggatttaag gacaatgtct atcgacagag aagaaagtat |
| 721 | tttgtggatg tggccatggg ttataaatat ggtcagccca ttcccagggt ggagtatact |
| 781 | gaagaagaaa ctaaaacttg gggtgttgta ttccgggagc tctccaaact ctatcccact |
| 841 | catgcttgcc gagagtattt gaaaaacttc cctctgctga ctaaatactg tggctacaga |
| 901 | gaggacaatg tgcctcaact cgaagatgtc tccatgtttc tgaaagaaag gtctggcttc |
| 961 | acggtgaggc cggtggctgg atacctgagc ccacgagact ttctggcagg actggcctac |
| 1021 | agagtgttcc actgtaccca gtacatccgg catggctcag atcccctcta caccccagaa |
| 1081 | ccagacacat gccatgaact cttgggacat gttccactac ttgcggatcc taagtttgct |
| 1141 | cagttttcac aagaaatagg tctggcgtct ctgggagcat cagatgaaga tgttcagaaa |
| 1201 | ctagccacgt gctatttctt cacaatcgag tttggccttt gcaagcaaga agggcaactg |
| 1261 | cgggcatatg gagcaggact cctttcctcc attggagaat taaagcacgc cctttctgac |
| 1321 | aaggcatgtg tgaaagcctt tgacccaaag acaacttgct tacaggaatg ccttatcacc |
| 1381 | accttccagg aagcctactt tgtttcagaa agttttgaag aagccaaaga aaagatgagg |
| 1441 | gactttgcaa agtcaattac ccgtcccttc tcagtatact tcaatcccta cacacagagt |
| 1501 | attgaaattc tgaaagacac cagaagtatt gaaaatgtgg tgcaggacct tcgcagcgac |
| 1561 | ttgaatacag tgtgtgatgc tttaaacaaa atgaaccaat atctggggat ttgatgcctg |
| 1621 | gaactatgtt gttgccagca tgatcttttt ggggcttagc agcagttcag tcaatgtcat |
| 1681 | ataacgcaaa taaccttctg tgtcatggct tggctaataa gcatgcaatt ccatatatct |
| 1741 | ataccatctt gtaactcact gtgttagtat ataaagcacc ataagaaatc caatggcaga |
| 1801 | taaccactca ttgtatgaaa taacgtatta tgtttaaaca tcttaaaaag atttgacatt |
| 1861 | cctgcttagt gtccttaacc aaactgcatc tagttaaaat ttgtaacaaa tagccctctt |
| 1921 | atgagtctca tttatgccct tttctttttc agatctaagc ctttcctctg tgttcattag |
| 1981 | ataaaatgaa aaaaagcagt gaagctgttt ccattttcaa tagtatcagt gttttcacgc |
| 2041 | attatttgag ataaacccag aattgtagga aacttcccat cacaataaca aaggttcaat |
| 2101 | attctatttc aaaaattgtt gaggtaacac agcagttgga atgattttta ggttgagtat |
| 2161 | ttacacaatg caagaaaaca cctttttaca aatggaatta tgtaggttgc gttgaccttg |
| 2221 | tagaacctga gttatgacaa gcttcctgaa gtattttgga agatagtact tccggaaagg |
| 2281 | acattaggaa agactaaaca gtggacaatc aatcttggga ctatgaattt tatgctggaa |
| 2341 | taaagtaaat tatcatgttc |
| TPH2 Protein Sep | |
| 1 | mqpammmfss kywarrgfsl dsavpeehql lgsstlnkpn sgknddkgnk gsskreaate |
| 61 | sgktavvfsl knevgglvka lrlfqekrvn mvhiesrksr rrsseveifv dcecgktefn |
| 121 | eliqllkfqt tivtlnppen iwteeeeled vpwfprkise ldkcshrvlm ygseldadhp |
| 181 | gfkdnvyrqr rkyfvdvamg ykygqpiprv eyteeetktw gvvfrelskl ypthacreyl |
| 241 | knfplltkyc gyrednvpql edvsmflker sgftvrpvag ylsprdflag layrvfhctq |
| 301 | yirhgsdply tpepdtchel lghvplladp kfaqfsqeig laslgasded vqklatcyff |
| 361 | tiefglckqe gqlraygagl lssigelkha lsdkacvkaf dpkttclqec littfqeayf |
| 421 | vsesfeeake kmrdfaksit rpfsvyfnpy tqsieilkdt rsienvvqdl rsdlntvcda |
| 481 | lnkmnqylgi |
| HUMAN NEUROGENIN 3 | |
| GENE SEQ (Genbank Accession No. NG_021321 (SEO ID NO. 1) | |
| MRNA SEQ | |
| 1 | cgcgatctgc tgcagctcgg ccgggagacg gcgcgacccg gcggcggggc cacccgcgag |
| 61 | tccagcgtcg ccgcagcccc ccaatgcggc cgcgagaagc agcggggggg caggcgatcg |
| 121 | aaggagcctt cacgtaaatg ggtccagtca tgcctcccag taagaagcca gaaagctcag |
| 181 | gaattagtgt ctccagtgga ctgagtcagt gttacggggg cagcggtttc tccaaggccc |
| 241 | ttcaggaaga cgatgacctc gacttttctc tgcctgacat ccgattagaa gagggggcca |
| 301 | tggaagatga agagctgacc aacctgaact ggctgcacga gagcaagaac ttgctgaaga |
| 361 | gctttgggga gtcggtcctc aggagtgtca gccccgtcca ggacctggac gatgacaccc |
| 421 | ccccatcccc tgcccactct gacatgccct acgatgccag gcagaacccc aactgcaaac |
| 481 | ccccctactc cttcagctgc ctcatattta tggccatcga ggactctcca accaagcgcc |
| 541 | tgccagtgaa ggatatctac aactggatct tggaacattt tccgtatttt gcaaatgcac |
| 601 | ctactgggtg gaaaaactca gtgagacaca atttatcatt gaataagtgt tttaagaaag |
| 661 | tggacaaaga gaggagtcag agtattggga aagggtcgtt gtggtgcata gacccagagt |
| 721 | atagacaaaa tctaattcag gctttgaaaa agacacctta tcacccacac ccacacgtgt |
| 781 | tcaatacacc tcccacctgt cctcaggcat atcaaagcac atcaggtcca cccatctggc |
| 841 | cgggcagtac cttcttcaag agaaatggag cccttctcca agatcctgac attgatgctg |
| 901 | ccagtgccat gatgcttttg aatactcccc ctgagataca agcaggtttt cctccaggag |
| 961 | tgatccaaaa tggagcgcgg gtcctgagcc gagggctgtt tcctggcgtg cggccgctgc |
| 1021 | caatcactcc cattggggtg acagcggcca tgaggaatgg catcaccagc tgccggatgc |
| 1081 | ggactgagag tgagccatct tgtggctccc cagtggtcag cggagacccc aaggaggatc |
| 1141 | acaactacag cagtgccaag tcctccaacg cccggagcac ctcgcccacc agcgactcca |
| 1201 | tctcctcctc ctcctcctca gccgacgacc actatgagtt tgccaccaag gggagccagg |
| 1261 | agggcagcga gggcagcgag gggagcttcc ggagccacga gagccccagc gacacggaag |
| 1321 | aggacgacag gaagcacagc cagaaggagc ccaaggattc tctgggggac agcgggtacg |
| 1381 | catcccagca caagaagcgc cagcacttcg ccaaggccag gaaggtcccc agcgacacac |
| 1441 | tgcccctcaa aaagagacgc accgaaaagc cccccgagag cgatgatgag gagatgaaag |
| 1501 | aagcggcagg gtccctcctg cacttagcag ggatccggtc ctgtttgaat aacatcacca |
| 1561 | atcggacggc aaaggggcag aaagagcaaa aggaaaccac aaaaaattaa aaacaagtca |
| 1621 | ctgatttgtt ttgaacttac gaccatttgg tttcagcatg tcaggagatt tctaatgatt |
| 1681 | tgtggcaata tcagcaattt tttttctttt ttcttgtttt tggtttggtt ttctttcttt |
| 1741 | tcttttcctt ttattttgtt ttaatttgcc ccctcttctt tgttttggac ccttaagaat |
| 1801 | tttattttta aaggagattg aagccataga actcatattg acactcagct gttttacaaa |
| 1861 | agcttttcat tatctgaaga caaaaccgaa aaagccaaaa ttaccattgc ttcctccagc |
| 1921 | ttgtcagaaa cctgtggctg aatccgcagg gatgtcaacg tcaatatcac aggaacacac |
| 1981 | attcggcacc tagaaggcac gtgggcaaag taatcatcgt tcaggcccaa cccttaggtt |
| 2041 | taaaaagtca ggttgtccat cccattgggt tcactgagtg aaggcacata aagcaattga |
| 2101 | ggaggaggag gaacccctcg tccccctagg agcagaccca agcttgtggc accaggcatc |
| 2161 | tgatggtgcc aggaaagcca ctggaattgt cacacggcga gcacagaggg ccggccacca |
| 2221 | gtcctcgatg cttctgaacc ctgaagcccg atgacatctt acgaggtgga cgttggactg |
| 2281 | ttcatgcgca tcgggtgtca gtgactcatg gagaagaaat ggggtaaatt tttagtgatg |
| 2341 | ttgctaatca ttgaattctg ttctctatta aattaagaaa atgttccaaa agccataagc |
| 2401 | ctgaagattg gccctgtgca cgcacgcaca cacacacaca cacacacaca cacacacaca |
| 2461 | cacacacgaa ggagagagag agaaaactga tggggaaaac aagctgtgtc ttcttaactg |
| 2521 | cccaagtgaa aagcaaccaa gtccaggaaa ttacaatagc tgttaaggaa aggaaataat |
| 2581 | ggtacagatc tttttctgtc tatcaaaact atttgatcca agtgaaaaaa aaaaaaaaac |
| 2641 | tagaaagcta cggaacctgc cattagtatt gtggtgtatt tttaagatta aaggtacact |
| 2701 | gatggacaaa aaaaaaaagt aaaacatggc aaaaaataaa ataactccta tactgccctc |
| 2761 | aaaatggagt ttgcaattaa tatcaggatt tatctttgca aaaatcagtg atttccacat |
| 2821 | tcagccagta tagccagcag aaatttctga tccacaatgc atggattcct ttgaagaaaa |
| 2881 | aaaagaaaaa gagaaaaaaa tcacaaaaac aaactttttt tattcaaaag taacaaagtt |
| 2941 | cttgtaaggt aaataatgta tttagcatga agcatgaatt attttcatat aaatatagaa |
| 3001 | aatagagaaa aggctatgcc tgtaattttt aagcccttag gcttagagtt tcttttggtt |
| 3061 | ttcttctttt ttctttcctt ttctttgctt tctttttttc ctttttgttt ttgtttttgt |
| 3121 | tttttgtttt tgtttttttt tcgggttatt ttgttttggt tttttgaagc aggtgtttaa |
| 3181 | ggtttaacct tcttcaggga caaattctga ctgttgggga acttactctg caatataaaa |
| 3241 | atatcttcat gctctggtag ggcttggatg gttgaactct gtactgcctt gtgtgcactt |
| 3301 | cagccccgac cccctctgat tctctgttga aaagtgtgtc ctttctctct gtctgtacat |
| 3361 | gtttaacatg acgcaataat ttgagggcaa acttagtagt gagtgtgtat gatagaatca |
| 3421 | agagaattat gggacgctta cttgagaaaa tcattaccat gatttggttc taggaaaaag |
| 3481 | gcagtgaata attatgcaaa ttagccagaa gaaggggaac cgtgctaatg ggccttattg |
| 3541 | ggtgagggga cgagatgggg ttcatgtgaa ggaggaagcg atgccgaggt aggaaaggcc |
| 3601 | agccccagac atcctatcgc cacaatgcca tgtcgcaata ggaagcaggg gccggccatc |
| 3661 | gctaccttca gcacactgac caacctggaa ttaagaccac ctagattgcg agagctgaat |
| 3721 | ttagaaacca gacaacgtca tgcagcccag aaactcctgt tgttaccttt gcctaagaaa |
| 3781 | ttttctttaa tggcgggggc ggggggcggg ggtacaaaga gaaatctcta aaagaatatg |
| 3841 | atcttccatc caagtggagg gaaactttaa aacaaaaaca cccagtactg tggctcagga |
| 3901 | tatgatgcgt gaggagaggg agggaacaga gatgacctta acttttaaaa aagggactgc |
| 3961 | tgtgggccaa agccaagccc atctgccagg acgaggtaat gtcagagctc catcagcccg |
| 4021 | gacagtggga actaactggt gcattcccca cacttacctt ccggtgggtt gctgatgaga |
| 4081 | gaacctgaaa aaacctacac ctctacagca ggtcgaattc atgacctgaa gctgaatact |
| 4141 | tccagcatat ttattcaggg tgtaggtggg aataaagtat cttcgcagtg ctctgttccc |
| 4201 | tccgtctccc cagacatctg acaccctaaa agccatccac agctatggaa cctgagcgac |
| 4261 | accttgattt gtgttgtcac ctgaccaagc ctaaagacct ccagctcagt cccccacctt |
| 4321 | catcccaccc cacagatgat aaaattcaga cctctctcct gaaaggcaga ggttcaacat |
| 4381 | tcaggactgt ttctggccga ggacttcttc caattaaaac ccccaccgtg ggctgtctcc |
| 4441 | cctcatttca tttttctaaa ggggcagagg cctcttttag aaaataataa aatgcaatgt |
| 4501 | gtgtgattta cttttctgat ctctttgaga aatagagaaa tataaaagtg tgttcttaac |
| 4561 | tccagaacca ctctttttgc ataaatacct catcgggcag ctttctaagt gtgattttcc |
| 4621 | tgagtctccc ttcgttggat ctgccggaag acttgtcggg gaacctttag tgagggtact |
| 4681 | tcttcctatt tttcttctgt ttttggaggc atacacatta tgcataacca aaacaatggc |
| 4741 | tcaattgtgt ttaactttgt attttgattg ttgagaacaa aaacaaaaag tatcaatgtg |
| 4801 | tatgtggctg tttgtagtga atttattgga gaatgaggtt gtccgtgtcc ttaacaagcc |
| 4861 | aaggggcagg aggcaccctc tcttatcccc tcctccaaga gcagtagaga atttaagcac |
| 4921 | aagcctattt gtgaaagaat attttgctta agtgtcattc actttagtct tggaattcct |
| 4981 | tcccaaacgt caggtgttct tttagcttcc aaactagcat atgtatccat tagtctgaca |
| 5041 | gatcgcctga acaccattaa gaggtgtggc gtttttgctt tcatttctcc tgctgggaga |
| 5101 | agtggcggtt catgtgtcat tccagtatct cacatactca cacggggcag gggggagggg |
| 5161 | gaaacgggga actatagcaa tatttaaaga tgctttggaa accaaccgtg aacacatcaa |
| 5221 | caccacgacg tctacgatta cttgctattg gccctcggat acatttaaga gaaagagaca |
| 5281 | gtcactcttt tttttcttaa atgatataca tataaacagt tatttttatc ctattataat |
| 5341 | tgtcttttgt ctttatctag tactatgtgg aaagggtttg catcatagat ttttcccagc |
| 5401 | cttataatat accataagct cctacttccc tgcccctccc taatcagtat tctttcaaga |
| 5461 | gttctttggt gaagccatct atctgaaact aaaatgaacc aaacccatat ttcactggtg |
| 5521 | gttggagaaa accatggcca aaacgattgt ggcaggtctc aatcttggga gtttttaaga |
| 5581 | aggaatgtgc cagaggccga ttcccaagaa cagagttttc ttttgttttg cagaggcatt |
| 5641 | caatgtgtct agtgcttgct ggccacagca gttactacca cagagccttc tgggaggggc |
| 5701 | cgttgtgttg aaggaggctc ctgcctgagg gacagcatca ggcagtgggc tctgtagagt |
| 5761 | gagaaccagg tggaggcctt ctgtgcccag ctcagagttc tgcaccacgc caggactgcc |
| 5821 | caggccaagg gctactgacg caagttccac tcattccact ctgtgggggg cgccttgggc |
| 5881 | ctctcctgga agggctcttg gagaaggaat tggagttacg tacaagtgac ctaaatggga |
| 5941 | agcttttcta gatgagattg gattaaattc catgtgattt ctctttccct ttaatccagg |
| 6001 | ttgggactcg tttctttctg gtggatcaca gctgcccaga tgttgcaatt gatttttatg |
| 6061 | tttctgtaga gaagtatttt tctttcatct tcaggatttt ttttgccacc aaaagaaaac |
| 6121 | attggaactc tgtgtttcct cttgattgtg acttcccagt gttgacagtt aagtccttag |
| 6181 | tgtcgtaggt cccagcccac caatactata tcaaacactg ttatgcacat aatgcagcac |
| 6241 | tgtgatctaa tttaaataat acttttttat tatttatact actatatata atatacatca |
| 6301 | acacttttgc tatataacct aagtgataac cctcttttag ttacctgcca aactctggac |
| 6361 | ttggtttata ttgcagttaa cacagttaca aagctgtaat ggtgtctttt tttcctttgt |
| 6421 | aacggaatgt gtaaatcaaa gtatatacat tgtgtggtgt tcctgtttct ggagtttcat |
| 6481 | gaggatttac acatggcatt cagtgttctg tatagatctg cctacctttg tgaattcatc |
| 6541 | tgttaacccc tcttcctttg agagagcacc ggcgatggtg gttaactcct tgtgttttct |
| 6601 | ctctctccta ctggttattc ttgaattaag cacagactcg tcagctcggt tgctttatca |
| 6661 | tgaataatgt gtgtgacctt gcagttcttc cacagttcag caaacaagtg ctagcttcac |
| 6721 | tgaccaaaaa ttaaggaagg aaaacacagt ttttaaaacg atccatcttt taacagccga |
| 6781 | aaccgatgtg tctatggtgc tgcaccttgc tgttgtactt ctgaaatcag acgtgtgtga |
| 6841 | acgatcattt ctgacttaac cgtgagatgc tcacgagtac ccttcctgtt gttttgttag |
| 6901 | cattgaaatc gagactattt atttggaata tatacaacag tgtttttcca ctgtatttca |
| 6961 | tttgcaaaag ttgagaactg ctttctctac cttttgcaaa ataattgata ttccatattg |
| 7021 | gattctcaaa gacttcgata tggtgaacct attaaaccta gaaattgtat tcatcctttc |
| 7081 | atgactgtgg cctgagttcc ccagcccctc tcctcctttt ttttagatga gatttagcac |
| 7141 | actctcagtt atttaaacat gcaacatttc ttgagtatgt atgttgaggc catctgagct |
| 7201 | catagctgat tcagtaacca gtttcatgct gtgtcattca cactcactac ttaatactgc |
| 7261 | catggtgaaa atgtggagga aaaatgtatc catgtgtgtc tgggaagcat atacacttgt |
| 7321 | acatttttta atactctgat tctgtaacat ttctgagttt tgttttgttt tacagaaaaa |
| 7381 | aaaaaaaagt gataaagcaa tcagaagacc aagaggttta ctattgatgc ttagggtcgt |
| 7441 | ctgaccttgg ctggccaata gacctacacg gccaaattaa tttacgagag taataatttt |
| 7501 | tcaaaagcca attttttttc tgtattttct gtatgaaact gccaatatca tgaatagaaa |
| 7561 | gggagaacca taaaggagaa agaacgtgat gttctgttat gttcatgtaa acctaaagaa |
| 7621 | acagtgtgga ggcaggcgcg atcagccgaa ctctagggac ttggtgttgc ttggaaggca |
| 7681 | tccatacctg cattttgcat tcttcgtatg taatcatatt gccaaagaca aactatttca |
| 7741 | tcatttattg taaataacac ttttccccag acctaccata aagtttctgt gatgtattgt |
| 7801 | cttccagttg caataaaaat tactgagttg catcaattga agaaaaacac caaaaa |
| Neurogenin 3 protein sequence | |
| 1 | mgpvmppskk pessgisvss glsqcyggsg fskalqeddd ldfslpdirl eegamedeel |
| 61 | tnlnwlhesk nllksfgesv lrsvspvqdl dddtppspah sdmpydarqn pnckppysfs |
| 121 | clifmaieds ptkrlpvkdi ynwilehfpy fanaptgwkn svrhnlslnk cfkkvdkers |
| 181 | qsigkgslwc idpeyrqnli qalkktpyhp hphvfntppt cpqayqstsg ppiwpgstff |
| 241 | krngallqdp didaasamml lntppeiqag fppgviqnga rvlsrglfpg vrplpitpig |
| 301 | vtaamrngit scrmrtesep scgspvvsgd pkedhnyssa kssnarstsp tsdsisssss |
| 361 | saddhyefat kgsqegsegs egsfrshesp sdteeddrkh sqkepkdslg dsgyasqhkk |
| 421 | rqhfakarkv psdtlplkkr rtekppesdd eemkeaagsl lhlagirscl nnitnrtakg |
| 481 | qkeqkettkn |

### REFERENCES

1. Talchai, C., Xuan, S., Kitamura, T., Depinho, R. A., and Accili, D. 2012. Generation of functional insulin-producing cells in the gut by Foxo1 ablation. Nature genetics 44:406-412.
2. Blum, B., Hrvatin, S. S., Schuetz, C., Bonal, C., Rezania, A., and Melton, D. A. 2012. Functional beta-cell maturation is marked by an increased glucose threshold and by expression of urocortin 3. Nat Biotechnol 30:261-264.
3. Schulz, T. C., Young, H. Y., Agulnick, A. D., Babin, M. J., Baetge, E. E., Bang, A. G., Bhoumik, A., Cepa, I., Cesario, R. M., Haakmeester, C., et al. 2012. A scalable system for production of functional pancreatic progenitors from human embryonic stem cells. PLoS One 7:e37004.
4. Hua, H., Shang, L., Martinez, H., Freeby, M., Gallagher, M. P., Ludwig, T., Deng, L., Greenberg, E., Leduc, C., Chung, W. K., et al. 2013. iPSC-derived beta cells model diabetes due to glucokinase deficiency. J Clin Invest 123:3146-3153.
5. Schonhoff, S. E., Giel-Moloney, M., and Leiter, A. B. 2004. Minireview: Development and differentiation of gut endocrine cells. Endocrinology 145:2639-2644.
6. Tu, J., Khoury, P., Williams, L., and Tuch, B. E. 2004. Comparison of fetal porcine aggregates of purified beta-cells versus islet-like cell clusters as a treatment of diabetes. Cell Transplant 13:525-534.

1. An insulin-negative cell wherein at least one genomic target gene selected from the group consisting of Neurogenin 3, TPH2, TPH1, Foxo1 and insulin is genetically modified by fusion to a reporter gene such that expression of the reporter gene is a readout of expression of the target gene.

2. The cell of claim 1, wherein mRNA encoding the fused gene is in a single reading frame.

3. The cell of claim 3, wherein mRNA encoding the fused gene is in a two reading frames.

4. The cell of claim 1, wherein two or more genomic target genes are genetically modified, each with a different fluorescent reporter gene.

5. The cell of claim 1, wherein the cell is a stem cell or progenitor cell, a Neurogenin 3 positive cell, a foxo1 positive cell, a Tph1 positive cell or a Tph2 positive cell.

6. The cell of claim 1, wherein the cell is a gut cell or a pancreatic cell.

7. The cell of claim 1, wherein the reporter gene is fused to exon 1 of the target gene, or to the last coding exon of the target gene before a stop codon.

8. The cell of claim 1, wherein the fluorescent reporter gene is introduced into the cells in by homologous recombination at a double stranded DNA break.

9. The cell of claim 1, wherein the genetic modification is made using a Clustered Regularly Interspaced Short Palindromic Repeats (CR/SPR)-associated protein method that implements a Cas protein.

10. The cell of claim 8, wherein the double stranded DNA break and the genetic modification is made using a Clustered Regularly Interspaced Short Palindromic Repeats (CR/SPR)-associated protein method that implements a Cas protein.

11. The cell of claim 9, wherein the Cas protein is Cas9.

12. The cell of claim 9, wherein the CR/SPR-associated method comprises introducing into the cell: (i) a first expression construct comprising a first promoter operably linked to a first nucleic acid sequence encoding a CR/SPR-associated (Cas) protein, and (ii) a second expression construct comprising a second promoter operably linked to a second nucleic acid sequence encoding a genomic RNA (gRNA) sequence complementary to a first particular genomic target sequence.

13. The cell of claim 1, wherein the genomic target sequence is immediately flanked on the 3′ end by a Protospacer Adjacent Motif (PAM) sequence in the genome.

14. The cell of claim 12, wherein the gRNA comprises a nucleic acid sequence encoding a Clustered Regularly Interspaced Short Palindromic Repeats (CRISPR) RNA (crRNA) and a trans-activating CRISPR RNA (tracrRNA).

15. The cell of claim 12, wherein the Cas makes a double-stranded DNA break in the genome.

16. The cell of claim 12, wherein the CRISPR method further comprises (iii) introducing into the cell a large targeting vector (LTVEC), comprising a first gene encoding a first fluorescent reporter targeted to a first target gene that is immediately flanked on the 3′ end by a Protospacer Adjacent Motif (PAM) sequence, selected from the group consisting of Neurogenin 3, TPH2, TPH1, FOXO1, and insulin.

17. A method for targeted modification of at least one genomic target gene selected from the group consisting of Neurogenin 3, TPH2, TPH1, Foxo1, and insulin in a mammalian stem cell or pluripotent cell, multipotent cell, or partially or terminally differentiated cell comprising introducing to the cell (i) a first expression construct comprising a first promoter operably linked to a first nucleic acid sequence encoding a CRISPR- associated (Cas) protein, and (ii) a second expression construct comprising a second promoter operably linked to a second nucleic acid sequence encoding a guide RNA (gRNA) sequence comprising a sequence that is complementary to a first target sequence in the genome that is immediately flanked on the 3′ end by a Protospacer Adjacent Motif (PAM) sequence linked to a guide RNA (gRNA).

18. The method of claim 17, further comprising (iii) introducing into the cell an expression construct (cassette), comprising a gene encoding a fluorescent reporter gene to be fused to a genomic target gene.

19. The method of claim 13, wherein the expression construct comprises a 5′ homology arm and a 3′ homology arm flanking the fluorescent reporter gene.

20. The method of claim 17 and the cell of claim 1, wherein the gene modifications are capable of being transmitted through the germline.

21. A method for identifying an agent that modulates expression in a cell of at least one genetically modified genomic target gene selected from the group consisting of Neurogenin 3, TPH2, TPH1, FOXO1, and insulin, which target gene is fused to a fluorescent reporter gene such that expression of the reporter gene is a readout of expression of the target gene, comprising (i) culturing the cell under conditions that permit target gene expression indicated by detectable fluorescence from the reporter gene, (ii) contacting the cell with a test agent in an amount and for a duration of time that permits the test agent to modulate target gene expression in the cell, and (iii) selecting the test agent if it modulates target gene expression, indicated by a change of in the amount of the fluorescence in the cell.

22. The method of claim 21 wherein the test agent reduces expression.

23. The method of claim 22 wherein the test agent increases expression.

24. The method of claim 21, wherein the cell is modified to express at least two target genes each fused to a different fluorescent marker and selecting the agent if it produces a loss of fluorescence of one of or both of the different fluorescent markers, or a change of color indicating an overlap of fluorescence from the different fluorescent markers.

25. The method of claim 22, wherein the fluorescent reporter gene is fused to an end of the target gene either before or after the target gene.

26. The method of claim 25, wherein the fluorescent reporter gene is placed before a stop codon in the target gene.

27. The method of claim 21, wherein the cell is a plurality of cells.

28. The method of claim 27, wherein the plurality of cells in a monolayer of cells on a substrate.

29. The method of claim 27, wherein the plurality of cells is a gut organoid.

30. The cell of claim 1, wherein the genomic target gene is TPH2.

31. The cell of claim 1, wherein the genomic target gene is insulin.

32. An insulin-negative gut cell genetically modified to comprise a reporter gene fused to a TPH2 gene or insulin gene such that expression of the reporter gene occurs with expression of TPH2 or insulin.

33. The insulin-negative cell of claim 1, wherein the reporter gene is fused within 10 bp upstream of a protospacer adjacent motif (PAM) sequence on the target gene.

34. An insulin-negative cell wherein at least one genomic target gene selected from the group consisting of Neurogenin 3, TPH2, TPH1, Foxo1 and insulin is genetically modified by fusion to a reporter gene such that expression of the reporter gene is a readout of expression of the target gene, wherein the genomic target sequence is immediately flanked on the 3′ end by a Protospacer Adjacent Motif (PAM) sequence in the genome.


SEQUENCE LISTING (30 sequences)

SEQ ID NO: 1 - DNA, 7856 bp, Homo sapiens
  misc_feature (1)..(7856): Human neurogenin 3
cgcgatctgctgcagctcggccgggagacggcgcgacccggcggcggggccacccgcgagtccagcgtcgccgcagccccccaatgcggccgcgagaagcagcgggggggcaggcgatcgaaggagccttcacgtaaatgggtccagtcatgcctcccagtaagaagccagaaagctcaggaattagtgtctccagtggactgagtcagtgttacgggggcagcggtttctccaaggcccttcaggaagacgatgacctcgacttttctctgcctgacatccgattagaagagggggccatggaagatgaagagctgaccaacctgaactggctgcacgagagcaagaacttgctgaagagctttggggagtcggtcctcaggagtgtcagccccgtccaggacctggacgatgacacccccccatcccctgcccactctgacatgccctacgatgccaggcagaaccccaactgcaaacccccctactccttcagctgcctcatatttatggccatcgaggactctccaaccaagcgcctgccagtgaaggatatctacaactggatcttggaacattttccgtattttgcaaatgcacctactgggtggaaaaactcagtgagacacaatttatcattgaataagtgttttaagaaagtggacaaagagaggagtcagagtattgggaaagggtcgttgtggtgcatagacccagagtatagacaaaatctaattcaggctttgaaaaagacaccttatcacccacacccacacgtgttcaatacacctcccacctgtcctcaggcatatcaaagcacatcaggtccacccatctggccgggcagtaccttcttcaagagaaatggagcccttctccaagatcctgacattgatgctgccagtgccatgatgcttttgaatactccccctgagatacaagcaggttttcctccaggagtgatccaaaatggagcgcgggtcctgagccgagggctgtttcctggcgtgcggccgctgccaatcactcccattggggtgacagcggccatgaggaatggcatcaccagctgccggatgcggactgagagtgagccatcttgtggctccccagtggtcagcggagaccccaaggaggatcacaactacagcagtgccaagtcctccaacgcccggagcacctcgcccaccagcgactccatctcctcctcctcctcctcagccgacgaccactatgagtttgccaccaaggggagccaggagggcagcgagggcagcgaggggagcttccggagccacgagagccccagcgacacggaagaggacgacaggaagcacagccagaaggagcccaaggattctctgggggacagcgggtacgcatcccagcacaagaagcgccagcacttcgccaaggccaggaaggtccccagcgacacactgcccctcaaaaagagacgcaccgaaaagccccccgagagcgatgatgaggagatgaaagaagcggcagggtccctcctgcacttagcagggatccggtcctgtttgaataacatcaccaatcggacggcaaaggggcagaaagagcaaaaggaaaccacaaaaaattaaaaacaagtcactgatttgttttgaacttacgaccatttggtttcagcatgtcaggagatttctaatgatttgtggcaatatcagcaattttttttcttttttcttgtttttggtttggttttctttcttttcttttccttttattttgttttaatttgccccctcttctttgttttggacccttaagaattttatttttaaaggagattgaagccatagaactcatattgacactcagctgttttacaaaagcttttcattatctgaagacaaaaccgaaaaagccaaaattaccattgcttcctccagcttgtcagaaacctgtggctgaatccgcagggatgtcaacgtcaatatcacaggaacacacattcggcacctagaaggcacgtgggcaaagtaatcatcgttcaggcccaacccttaggtttaaaaagtcaggttgtccatcccattgggttcactgagtgaaggcacataaagcaattgaggaggaggaggaacccctcgtccccctaggagcagacccaagcttgtggcaccaggcatctgatggtgccaggaaagccactggaattgtcacacggcgagcacagagggccggccaccagtcctcgatgcttctgaaccctgaagcccgatgacatcttacgaggtggacgttggactgttcatgcgcatcgggtgtcagtgactcatggagaagaaatggggtaaatttttagtgatgttgctaatcattgaattctgttctctattaaattaagaaaatgttccaaaagccataagcctgaagattggccctgtgcacgcacgcacacacacacacacacacacacacacacacacacacacacgaaggagagagagagaaaactgatggggaaaacaagctgtgtcttcttaactgcccaagtgaaaagcaaccaagtccaggaaattacaatagctgttaaggaaaggaaataatggtacagatctttttctgtctatcaaaactatttgatccaagtgaaaaaaaaaaaaaaactagaaagctacggaacctgccattagtattgtggtgtatttttaagattaaaggtacactgatggacaaaaaaaaaaagtaaaacatggcaaaaaataaaataactcctatactgccctcaaaatggagtttgcaattaatatcaggatttatctttgcaaaaatcagtgatttccacattcagccagtatagccagcagaaatttctgatccacaatgcatggattcctttgaagaaaaaaaagaaaaagagaaaaaaatcacaaaaacaaactttttttattcaaaagtaacaaagttcttgtaaggtaaataatgtatttagcatgaagcatgaattattttcatataaatatagaaaatagagaaaaggctatgcctgtaatttttaagcccttaggcttagagtttcttttggttttcttcttttttctttccttttctttgctttctttttttcctttttgtttttgtttttgttttttgtttttgtttttttttcgggttattttgttttggttttttgaagcaggtgtttaaggtttaaccttcttcagggacaaattctgactgttggggaacttactctgcaatataaaaatatcttcatgctctggtagggcttggatggttgaactctgtactgccttgtgtgcacttcagccccgaccccctctgattctctgttgaaaagtgtgtcctttctctctgtctgtacatgtttaacatgacgcaataatttgagggcaaacttagtagtgagtgtgtatgatagaatcaagagaattatgggacgcttacttgagaaaatcattaccatgatttggttctaggaaaaaggcagtgaataattatgcaaattagccagaagaaggggaaccgtgctaatgggccttattgggtgaggggacgagatggggttcatgtgaaggaggaagcgatgccgaggtaggaaaggccagccccagacatcctatcgccacaatgccatgtcgcaataggaagcaggggccggccatcgctaccttcagcacactgaccaacctggaattaagaccacctagattgcgagagctgaatttagaaaccagacaacgtcatgcagcccagaaactcctgttgttacctttgcctaagaaattttctttaatggcgggggcggggggcgggggtacaaagagaaatctctaaaagaatatgatcttccatccaagtggagggaaactttaaaacaaaaacacccagtactgtggctcaggatatgatgcgtgaggagagggagggaacagagatgaccttaacttttaaaaaagggactgctgtgggccaaagccaagcccatctgccaggacgaggtaatgtcagagctccatcagcccggacagtgggaactaactggtgcattccccacacttaccttccggtgggttgctgatgagagaacctgaaaaaacctacacctctacagcaggtcgaattcatgacctgaagctgaatacttccagcatatttattcagggtgtaggtgggaataaagtatcttcgcagtgctctgttccctccgtctccccagacatctgacaccctaaaagccatccacagctatggaacctgagcgacaccttgatttgtgttgtcacctgaccaagcctaaagacctccagctcagtcccccaccttcatcccaccccacagatgataaaattcagacctctctcctgaaaggcagaggttcaacattcaggactgtttctggccgaggacttcttccaattaaaacccccaccgtgggctgtctcccctcatttcatttttctaaaggggcagaggcctcttttagaaaataataaaatgcaatgtgtgtgatttacttttctgatctctttgagaaatagagaaatataaaagtgtgttcttaactccagaaccactctttttgcataaatacctcatcgggcagctttctaagtgtgattttcctgagtctcccttcgttggatctgccggaagacttgtcggggaacctttagtgagggtacttcttcctatttttcttctgtttttggaggcatacacattatgcataaccaaaacaatggctcaattgtgtttaactttgtattttgattgttgagaacaaaaacaaaaagtatcaatgtgtatgtggctgtttgtagtgaatttattggagaatgaggttgtccgtgtccttaacaagccaaggggcaggaggcaccctctcttatcccctcctccaagagcagtagagaatttaagcacaagcctatttgtgaaagaatattttgcttaagtgtcattcactttagtcttggaattccttcccaaacgtcaggtgttcttttagcttccaaactagcatatgtatccattagtctgacagatcgcctgaacaccattaagaggtgtggcgtttttgctttcatttctcctgctgggagaagtggcggttcatgtgtcattccagtatctcacatactcacacggggcaggggggagggggaaacggggaactatagcaatatttaaagatgctttggaaaccaaccgtgaacacatcaacaccacgacgtctacgattacttgctattggccctcggatacatttaagagaaagagacagtcactcttttttttcttaaatgatatacatataaacagttatttttatcctattataattgtcttttgtctttatctagtactatgtggaaagggtttgcatcatagatttttcccagccttataatataccataagctcctacttccctgcccctccctaatcagtattctttcaagagttctttggtgaagccatctatctgaaactaaaatgaaccaaacccatatttcactggtggttggagaaaaccatggccaaaacgattgtggcaggtctcaatcttgggagtttttaagaaggaatgtgccagaggccgattcccaagaacagagttttcttttgttttgcagaggcattcaatgtgtctagtgcttgctggccacagcagttactaccacagagccttctgggaggggccgttgtgttgaaggaggctcctgcctgagggacagcatcaggcagtgggctctgtagagtgagaaccaggtggaggccttctgtgcccagctcagagttctgcaccacgccaggactgcccaggccaagggctactgacgcaagttccactcattccactctgtggggggcgccttgggcctctcctggaagggctcttggagaaggaattggagttacgtacaagtgacctaaatgggaagcttttctagatgagattggattaaattccatgtgatttctctttccctttaatccaggttgggactcgtttctttctggtggatcacagctgcccagatgttgcaattgatttttatgtttctgtagagaagtatttttctttcatcttcaggattttttttgccaccaaaagaaaacattggaactctgtgtttcctcttgattgtgacttcccagtgttgacagttaagtccttagtgtcgtaggtcccagcccaccaatactatatcaaacactgttatgcacataatgcagcactgtgatctaatttaaataatacttttttattatttatactactatatataatatacatcaacacttttgctatataacctaagtgataaccctcttttagttacctgccaaactctggacttggtttatattgcagttaacacagttacaaagctgtaatggtgtctttttttcctttgtaacggaatgtgtaaatcaaagtatatacattgtgtggtgttcctgtttctggagtttcatgaggatttacacatggcattcagtgttctgtatagatctgcctacctttgtgaattcatctgttaacccctcttcctttgagagagcaccggcgatggtggttaactccttgtgttttctctctctcctactggttattcttgaattaagcacagactcgtcagctcggttgctttatcatgaataatgtgtgtgaccttgcagttcttccacagttcagcaaacaagtgctagcttcactgaccaaaaattaaggaaggaaaacacagtttttaaaacgatccatcttttaacagccgaaaccgatgtgtctatggtgctgcaccttgctgttgtacttctgaaatcagacgtgtgtgaacgatcatttctgacttaaccgtgagatgctcacgagtacccttcctgttgttttgttagcattgaaatcgagactatttatttggaatatatacaacagtgtttttccactgtatttcatttgcaaaagttgagaactgctttctctaccttttgcaaaataattgatattccatattggattctcaaagacttcgatatggtgaacctattaaacctagaaattgtattcatcctttcatgactgtggcctgagttccccagcccctctcctccttttttttagatgagatttagcacactctcagttatttaaacatgcaacatttcttgagtatgtatgttgaggccatctgagctcatagctgattcagtaaccagtttcatgctgtgtcattcacactcactacttaatactgccatggtgaaaatgtggaggaaaaatgtatccatgtgtgtctgggaagcatatacacttgtacattttttaatactctgattctgtaacatttctgagttttgttttgttttacagaaaaaaaaaaaaagtgataaagcaatcagaagaccaagaggtttactattgatgcttagggtcgtctgaccttggctggccaatagacctacacggccaaattaatttacgagagtaataatttttcaaaagccaattttttttctgtattttctgtatgaaactgccaatatcatgaatagaaagggagaaccataaaggagaaagaacgtgatgttctgttatgttcatgtaaacctaaagaaacagtgtggaggcaggcgcgatcagccgaactctagggacttggtgttgcttggaaggcatccatacctgcattttgcattcttcgtatgtaatcatattgccaaagacaaactatttcatcatttattgtaaataacacttttccccagacctaccataaagtttctgtgatgtattgtcttccagttgcaataaaaattactgagttgcatcaattgaagaaaaacaccaaaaa

SEQ ID NO: 2 - DNA, 2360 bp, Homo sapiens
  misc_feature (1)..(2360): Human TPH2
cattgctcttcagcaccagggttctggacagcgccccaagcaggcagctgatcgcacgccccttcctctcaatctccgccagcgctgctactgcccctctagtaccccctgctgcagagaaagaatattacaccgggatccatgcagccagcaatgatgatgttttccagtaaatactgggcacggagagggttttccctggattcagcagtgcccgaagagcatcagctacttggcagctcaacactaaataaacctaactctggcaaaaatgacgacaaaggcaacaagggaagcagcaaacgtgaagctgctaccgaaagtggcaagacagcagttgttttctccttgaagaatgaagttggtggattggtaaaagcactgaggctctttcaggaaaaacgtgtcaacatggttcatattgaatccaggaaatctcggcgaagaagttctgaggttgaaatctttgtggactgtgagtgtgggaaaacagaattcaatgagctcattcagttgctgaaatttcaaaccactattgtgacgctgaatcctccagagaacatttggacagaggaagaagagctagaggatgtgccctggttccctcggaagatctctgagttagacaaatgctctcacagagttctcatgtatggttctgagcttgatgctgaccacccaggatttaaggacaatgtctatcgacagagaagaaagtattttgtggatgtggccatgggttataaatatggtcagcccattcccagggtggagtatactgaagaagaaactaaaacttggggtgttgtattccgggagctctccaaactctatcccactcatgcttgccgagagtatttgaaaaacttccctctgctgactaaatactgtggctacagagaggacaatgtgcctcaactcgaagatgtctccatgtttctgaaagaaaggtctggcttcacggtgaggccggtggctggatacctgagcccacgagactttctggcaggactggcctacagagtgttccactgtacccagtacatccggcatggctcagatcccctctacaccccagaaccagacacatgccatgaactcttgggacatgttccactacttgcggatcctaagtttgctcagttttcacaagaaataggtctggcgtctctgggagcatcagatgaagatgttcagaaactagccacgtgctatttcttcacaatcgagtttggcctttgcaagcaagaagggcaactgcgggcatatggagcaggactcctttcctccattggagaattaaagcacgccctttctgacaaggcatgtgtgaaagcctttgacccaaagacaacttgcttacaggaatgccttatcaccaccttccaggaagcctactttgtttcagaaagttttgaagaagccaaagaaaagatgagggactttgcaaagtcaattacccgtcccttctcagtatacttcaatccctacacacagagtattgaaattctgaaagacaccagaagtattgaaaatgtggtgcaggaccttcgcagcgacttgaatacagtgtgtgatgctttaaacaaaatgaaccaatatctggggatttgatgcctggaactatgttgttgccagcatgatctttttggggcttagcagcagttcagtcaatgtcatataacgcaaataaccttctgtgtcatggcttggctaataagcatgcaattccatatatctataccatcttgtaactcactgtgttagtatataaagcaccataagaaatccaatggcagataaccactcattgtatgaaataacgtattatgtttaaacatcttaaaaagatttgacattcctgcttagtgtccttaaccaaactgcatctagttaaaatttgtaacaaatagccctcttatgagtctcatttatgcccttttctttttcagatctaagcctttcctctgtgttcattagataaaatgaaaaaaagcagtgaagctgtttccattttcaatagtatcagtgttttcacgcattatttgagataaacccagaattgtaggaaacttcccatcacaataacaaaggttcaatattctatttcaaaaattgttgaggtaacacagcagttggaatgatttttaggttgagtatttacacaatgcaagaaaacacctttttacaaatggaattatgtaggttgcgttgaccttgtagaacctgagttatgacaagcttcctgaagtattttggaagatagtacttccggaaaggacattaggaaagactaaacagtggacaatcaatcttgggactatgaattttatgctggaataaagtaaattatcatgttc

SEQ ID NO: 3 - DNA, 1815 bp, Homo sapiens
  misc_feature (1)..(1815): Human TPH1
ttttagagaattactccaaattcatcatgattgaagacaataaggagaacaaagaccattccttagaaaggggaagagcaagtctcattttttccttaaagaatgaagttggaggacttataaaagccctgaaaatctttcaggagaagcatgtgaatctgttacatatcgagtcccgaaaatcaaaaagaagaaactcagaatttgagatttttgttgactgtgacatcaacagagaacaattgaatgatatttttcatctgctgaagtctcataccaatgttctctctgtgaatctaccagataattttactttgaaggaagatggtatggaaactgttccttggtttccaaagaagatttctgacctggaccattgtgccaacagagttctgatgtatggatctgaactagatgcagaccatcctggcttcaaagacaatgtctaccgtaaacgtcgaaagtattttgcggacttggctatgaactataaacatggagaccccattccaaaggttgaattcactgaagaggagattaagacctggggaaccgtattccaagagctcaacaaactctacccaacccatgcttgcagagagtatctcaaaaacttacctttgctttctaaatattgtggatatcgggaggataatatcccacaattggaagatgtctccaactttttaaaagagcgtacaggtttttccatccgtcctgtggctggttacttatcaccaagagatttcttatcaggtttagcctttcgagtttttcactgcactcaatatgtgagacacagttcagatcccttctataccccagagccagatacctgccatgaactcttaggtcatgtcccgcttttggctgaacctagttttgcccaattctcccaagaaattggcttggcttctcttggcgcttcagaggaggctgttcaaaaactggcaacgtgctactttttcactgtggagtttggtctatgtaaacaagatggacagctaagagtctttggtgctggcttactttcttctatcagtgaactcaaacatgcactttctggacatgccaaagtaaagccctttgatcccaagattacctgcaaacaggaatgtcttatcacaacttttcaagatgtctactttgtatctgaaagttttgaagatgcaaaggagaagatgagagaatttaccaaaacaattaagcgtccatttggagtgaagtataatccatatacacggagtattcagatcctgaaagacaccaagagcataaccagtgccatgaatgagctgcagcatgatctcgatgttgtcagtgatgcccttgctaaggtcagcaggaagccgagtatctaacagtagccagtcatccaggaacatttgagcatcaattcggaggtctgggccatctcttgctttccttgaacacctgatcctggagggacagcatcttctggccaaacaatattatcgaattccactacttaaggaatcactagtctttgaaaatttgtacctggatattctatttaccacttatttttttgtttagttttatttctttttttttttggtagcagctttaatgagacaatttatataccatacaagccactgaccacccatttttaatagagaagttgtttgacccaatagatagatctaatctcagcctaactctattttccccaatcctccttgagtaaaatgaccctttaggatcgcttagaataacttgaggagtattatggcgctgactcatattgttacctaagatccccttatttctaaagtatctgttacttattgc

SEQ ID NO: 4 - DNA, 5738 bp, Homo sapiens
  misc_feature (1)..(5738): Human FOX01
gcagccgccacattcaacaggcagcagcgcagcgggcgcgccgctggggagagcaagcggcccgcggcgtccgtccgtccttccgtccgcggccctgtcagctggagcgcggcgcaggctctgccccggcccggcggctctggccggccgtccagtccgtgcggcggaccccgaggagcctcgatgtggatggccccgcgaagttaagttctgggctcgcgcttccactccgccgcgccttcctcccagtttccgtccgctcgccgcaccggcttcgttcccccaaatctcggaccgtcccttcgcgccccctccccgtccgcccccagtgctgcgttctccccctcttggctctcctgcggctgggggaggggcgggggtcaccatggccgaggcgcctcaggtggtggagatcgacccggacttcgagccgctgccccggccgcgctcgtgcacctggccgctgcccaggccggagtttagccagtccaactcggccacctccagcccggcgccgtcgggcagcgcggctgccaaccccgacgccgcggcgggcctgccctcggcctcggctgccgctgtcagcgccgacttcatgagcaacctgagcttgctggaggagagcgaggacttcccgcaggcgcccggctccgtggcggcggcggtggcggcggcggccgccgcggccgccaccggggggctgtgcggggacttccagggcccggaggcgggctgcctgcacccagcgccaccgcagcccccgccgcccgggccgctgtcgcagcacccgccggtgccccccgccgccgctgggccgctcgcggggcagccgcgcaagagcagctcgtcccgccgcaacgcgtggggcaacctgtcctacgccgacctcatcaccaaggccatcgagagctcggcggagaagcggctcacgctgtcgcagatctacgagtggatggtcaagagcgtgccctacttcaaggataagggtgacagcaacagctcggcgggctggaagaattcaattcgtcataatctgtccctacacagcaagttcattcgtgtgcagaatgaaggaactggaaaaagttcttggtggatgctcaatccagagggtggcaagagcgggaaatctcctaggagaagagctgcatccatggacaacaacagtaaatttgctaagagccgaagccgagctgccaagaagaaagcatctctccagtctggccaggagggtgctggggacagccctggatcacagttttccaaatggcctgcaagccctggctctcacagcaatgatgactttgataactggagtacatttcgccctcgaactagctcaaatgctagtactattagtgggagactctcacccattatgaccgaacaggatgatcttggagaaggggatgtgcattctatggtgtacccgccatctgccgcaaagatggcctctactttacccagtctgtctgagataagcaatcccgaaaacatggaaaatcttttggataatctcaaccttctctcatcaccaacatcattaactgtttcgacccagtcctcacctggcaccatgatgcagcagacgccgtgctactcgtttgcgccaccaaacaccagtttgaattcacccagcccaaactaccaaaaatatacatatggccaatccagcatgagccctttgccccagatgcctatacaaacacttcaggacaataagtcgagttatggaggtatgagtcagtataactgtgcgcctggactcttgaaggagttgctgacttctgactctcctccccataatgacattatgacaccagttgatcctggggtagcccagcccaacagccgggttctgggccagaacgtcatgatgggccctaattcggtcatgtcaacctatggcagccaggcatctcataacaaaatgatgaatcccagctcccatacccaccctggacatgctcagcagacatctgcagttaacgggcgtcccctgccccacacggtaagcaccatgccccacacctcgggtatgaaccgcctgacccaagtgaagacacctgtacaagtgcctctgccccaccccatgcagatgagtgccctggggggctactcctccgtgagcagctgcaatggctatggcagaatgggccttctccaccaggagaagctcccaagtgacttggatggcatgttcattgagcgcttagactgtgacatggaatccatcattcggaatgacctcatggatggagatacattggattttaactttgacaatgtgttgcccaaccaaagcttcccacacagtgtcaagacaacgacacatagctgggtgtcaggctgagggttagtgagcaggttacacttaaaagtacttcagattgtctgacagcaggaactgagagaagcagtccaaagatgtctttcaccaactcccttttagttttcttggttaaaaaaaaaaacaaaaaaaaaaaccctccttttttcctttcgtcagacttggcagcaaagacatttttcctgtacaggatgtttgcccaatgtgtgcaggttatgtgctgctgtagataaggactgtgccattggaaatttcattacaatgaagtgccaaactcactacaccatataattgcagaaaagattttcagatcctggtgtgctttcaagttttgtatataagcagtagatacagattgtatttgtgtgtgtttttggtttttctaaatatccaattggtccaaggaaagtttatactctttttgtaatactgtgatgggcctcatgtcttgataagttaaacttttgtttgtactacctgttttctgcggaactgacggatcacaaagaactgaatctccattctgcatctccattgaacagccttggacctgttcacgttgccacagaattcacatgagaaccaagtagcctgttatcaatctgctaaattaatggacttgttaaacttttggaaaaaaaaagattaaatgccagctttgtacaggtcttttctatttttttttgtttattttgttatttgcaaatttgtacaaacatttaaatggttctaatttccagataaatgatttttgatgttattgttgggacttaagaacatttttggaatagatattgaactgtaataatgttttcttaaaactagagtctactttgttacatagtcagcttgtaaattttgtggaaccacaggtatttggggcagcattcataattttcattttgtattctaactggattagtactaattttatacatgcttaactggtttgtacactttgggatgctacttagtgatgtttctgactaatcttaaatcattgtaattagtacttgcatattcaacgtttcaggccctggttgggcaggaaagtgatgtatagttatggacactttgcgtttcttatttaggataacttaatatgtttttatgtatgtattttaaagaaatttcatctgcttctactgaactatgcgtactgcatagcatcaagtcttctctagagacctctgtagtcctgggaggcctcataatgtttgtagatcagaaaagggagatctgcatctaaagcaatggtcctttgtcaaacgagggattttgatccacttcaccattttgagttgagctttagcaaaagtttcccctcataattctttgctcttgtttcagtccaggtggaggttggttttgtagttctgccttgaggaattatgtcaacactcatacttcatctcattctcccttctgccctgcagattagattacttagcacactgtggaagtttaagtggaaggagggaatttaaaaatgggacttgagtggtttgtagaatttgtgttcataagttcagatgggtagcaaatggaatagaacttacttaaaaattggggagatttatttgaaaaccagctgtaagttgtgcattgagattatgttaaaagccttggcttaagaatttgaaaatttctttagcctgtagcaacctaaactgtaattcctatcattatgttttattactttccaattacctgtaactgacagaccaaattaattggctttgtgtcctatttagtccatcagtattttcaagtcatgtggaaagcccaaagtcatcacaatgaagagaacaggtgcacagcactgttcctcttgtgttcttgagaaggatctaatttttctgtatatagcccacatcacacttgctttgtcttgtatgttaattgcatcttcattggcttggtatttcctaaatgtttaacaagaacacaagtgttcctgataagatttcctacagtaagccagctctattgtaagcttcccactgtgatgatcatttttttgaagattcattgaacagccaccactctatcatcctcattttggggcagtccaagacatagctggttttagaaacccaagttcctctaagcacagcctcccgggtatgtaactgaacttggtgccaaagtacttgtgtactaatttctattactacgtactgtcactttcctcccgtgccattactgcatcataatacaaggaacctcagagcccccatttgttcattaaagaggcaactacagccaaaatcactgttaaaatcttactacttcatggagtagctcttaggaaaatatatcttcctcctgagtctgggtaattatacctctcccaagcccccattgtgtgttgaaatcctgtcatgaatccttggtagctctctgagaacagtgaagtccagggaaaggcatctggtctgtctggaaagcaaacattatgtggcctctggtagtttttttcctgtaagaatactgactttctggagtaatgagtatatatcagttattgtacatgattgctttgtgaaatgtgcaaatgatatcacctatgcagccttgtttgatttattttctctggtttgtactgttattaaaagcatattgtattatagagctattcagatattttaaatataaagatgtattgtttccgtaatatagacgtatggaatatatttaggtaatagatgtattacttggaaagttctgctttgacaaactgacaaagtctaaatgagcacatgtatcccagtgagcagtaaatcaatggaacatcccaagaagaggataaggatgcttaaaatggaaatcattctccaacgatatacaaattggacttgttcaactgctggatatatgctaccaataaccccagccccaacttaaaattcttacattcaagctcctaagagttcttaatttataactaattttaaaagagaagtttcttttctggttttagtttgggaataatcattcattaaaaaaaatgtattgtggtttatgcgaacagaccaacctggcattacagttggcctctccttgaggtgggcacagcctggcagtgtggccaggggtggccatgtaagtcccatcaggacgtagtcatgcctcctgcatttcgctacccgagtttagtaacagtgcagattccacgttcttgttccgatactctgagaagtgcctgatgttgatgtacttacagacacaagaacaatctttgctataattgtataaagccataaatgtacataaattatgtttaaatggcttggtgtctttcttttctaattatgcagaataagctctttattaggaattttttgtgaagctattaaatacttgagttaagtcttgtcagccacaa

SEQ ID NO: 5 - DNA, 469 bp, Homo sapiens
  misc_feature (1)..(469): Human insulin
agccctccaggacaggctgcatcagaagaggccatcaagcagatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcccgcaggcagccccacacccgccgcctcctgcaccgagagagatggaataaagcccttgaaccagcaaaa

SEQ ID NO: 6 - DNA, 5631 bp, Artificial Sequence
  Synthetic Ngn3-EGFP-pA-Ngn3 1083 1Kb Arms
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagacagacagacttgagtgagggtagggcgacccaagacggtgggcggctccggccgggtagtgctaccattctagtattctttgaatgagattatggggtggtggcagagaggaggcctaaaatgagcgcactttgcaatgcccacttcgcgcgggcagcagcaagggttgcgtgcgttggcgcggctcggagggccggggaatgaacccagcctgccgcccccgtggaggcctgggccggccaggggtcagccagggagaagcagaaggaacaagtgcttttgagggccgccgccgtcggccaccctctacggctcccggctccctccctctcccttacccttagcacccacagcccagcgacagacaggtcctttcacagaaaatctcgagaaagccagactgcctgggctcaagcaggcggaagaggtggcccccagcagcccgggtcgctcctccagcgacgcggcgggactcaggctgccagcctgggagactggggagtagagggacccccagtccccgggggaaccgcctgggctgcccagctccccgcagtgcggcgccggcggctccagcgcgtacaagctgtggtccgctatgcgcagcgtttgagtcagcgcccagatgtagttgtgggcgaagcgcagcgtctcgatcttggtgagcttcgcgtcgtctgggaaggtgggcaggacaccgcgcagggcgtccagtgccgagttgaggttgtgcattcgattgcgctcgcggtcgttggccttctttcgccgactccgtcgctgcttgctcagtgccaactcgctcttaggccggctgcgtcccccgcgccgtgcccggagcttcctcggggcccctcggcagcctccctcttccgcctctgcgcagttcccccgtgtgcgagtggggctgggcggggcggacgtggggcaggtcacttcgtcttccgaggctctggggaaggaccgctccgtctcacgggtcacttggacagtgggcgcacccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagtcacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatccgagggttgaggcgtcatcctacggcggggtcagagggaagggtaagtttgagtccgtcactgggcgcagtccgcgattccgaggctaggtgggaaaaaacaaaaacagccatcctcccagcccccgctgggtcagaggatccctctttcccctgcccgtccctcggaggcctccaaatattacctttctaccggcgcaaaagaatagagagcgatgagcagcgagggccgtggggagctcagcgggcttctggtcgccaagttcagctgagctgcaggcgcccccgcctgggagttgccccagccccaaaggagaaaagaagagagaatggggtccgaggcctctgtcacgctctctctcgaggcgcggcggtgagaccgcagggatttcctgagcagcaagtcgtgtgccccttggcacgctttatctgcttcgcccgggccaggagcgtgcctgcccggctgctgcccgcgccaccggccaatcagcgccggggccctggggccgcgccacgcgagcccgctcctcccccgcagggcacagctggattccggacaaagggccggggtcgggggaggggagcgccgctctgtttgctctctcgagggcgggctgggtcccagcaactctcggttcctcaaagagcctcgcccagtgagaagagcctcgtgtggctctggtcaggccacctcagacggctttgctcctagcctatctttccttagcatctgtcctggaggggactttgatgcctctagggtacaatgcctgcacgttacacatggggaaatttaggcttagtgagggaggtggcttgtctgaaatcgcacaggaagatagtggcaaagacaaccacgagctcattgtcctgactagcagcctggagaagggtccaggaattctaaaggacgccctgctctcctggtgtttcactgcctctcttcatcctggaagacaggggacatcactgagagagatcctgcctatgtcccttccattgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 7 - DNA, 5738 bp, Artificial Sequence
  Synthetic Tph2-Cerulean-pA-Tph2 1083 1Kb Arms
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatccagtgaattcgagctcggtacctcgcgaatgcatctagacctttcctttgcaatacattttcctccatataactctgcatagaggcatcacaggattaagaagaagcccttttatgaaagccattacacatatatacactcacacatttgcatgcacaaaattagaatatgtcaagtcagaaaaagcttattaacataaaatggagttggtcaatgagtaaaaaaaatatgctgatgggagggataagatctagtgttcgggagcacaataatttattttcttttgtattttaaaataactggaagagtggaattggaatgtttctaacacaaaaagaaatgataaatgcttgaggcaatggatatcttgattaccttatttgatcattacacattgtacgcttgtgtcaaaatatcacatgtgccttataaatgtgtacaactattagttatccataaaaattaaaaattaaaaaatccgtaaaatggtttaagcattcagcagtgctgatctttcttaaattatttttctaattttggaaagaaagcacaaaatctttgaattcacaattgcttaaagactgaggttaacttgccagtggcaggcttgagagatgagagaactaacgtcagaggatagatggtttcttgtacaaataacacccccttatgtattgttctccaccacccccgcccaaaaagctactcgacctatgaaacaaatcacactatgagcacagataaccccaggcttcaggtctgtaatctgactgtggccatcggcaaccagaaatgagtttctttctaatcagtcttgcatcagtctccagtcattcatataaaggagcccggggatgggaggattcgcattgctcttcagcaccagggttctggacagcgccccaagcaggcagctgatcgcacgccccttcctctcaatctccgccagcgctgctactgcccctctagtaccccctgctgcagagaaagaatattacaccgggatccatgcagccagcaatgatgatgttttccagtaaatactgggcacggatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgactagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagagggttttccctggattcagcagtgcccgaagagcatcagctacttggcagctcaacagtgagtactacgtacctggcactatggagaattattttttagggtgtgaccatcttctcctcaccatatgaatcccttttgtagtgtaagcacgcacacctcaaatttctccttctttataatctgtctaccctgctttcctcctgtctgcctccagtcttcctcttctctccataagtaaagcgagtgtgccaatcactgcgtgctcaactttttttccgcaaagtttgtaagtagagagttaagaagttcctgaacattaagaatgagagattgtatgaatcaatgtcttaaatctacagccaaaaaaaaaaaaaaaaaaatggagtgtgaagaattttgaaaagccgtttattatgaggaggaggagtagggagaacaaattaaataaatttccacggttttcagaagatcattgtgtctcctacacccccttcagtttacaaagcctggtctttaaacatagaactattattttctcttcttagttatgggtgcaggttattggaataaaagaaagattggattcctttcaaaagtttttctgtgtttcacattgctcaatttttttcagtttacttgatggaataatgaaagcaatacaccacttgctatagtatttaagggagttttatgtttataatatctacaggataaaaaagcagtatttgcaggattttagatcctgctttcaggtagtagtcatgggatttaataaaaaccacgaaataaaaatgtatccaggtcctagtcattaaaaatattaaatggtattttattactgtactatcagagtttatcaaccaaatccaattcagtctgtatcatagaatcatctgttttaatttcgtagctccaaatatgtgccagagggctgcgttggactgacatattattactgataaaaatgttgaaaagtaaacatggcaacttctgtagagtcgactgcagaggcctgcatgcaagcttggcgtaatcatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 5622 bp, Artificial Sequence
  Synthetic Foxo1-mOrange-pA-Foxo1 1083 1Kb Arms
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagaagagaacccgccctccccccgcggaggtccgggagggaaggggcagccgaagcagtcggcgcgggccgggggttgccgctcccagcgaacccctttctcctttcactggcaaacttttcggcctcgctctgacgtccacttcttggcgcactttctttacttagttccccaacgagccccttaccgcgtcccacgcgaactcctgactggcgcgcacgcacacctactgccgtccccgaccggacccgggcgaggccaccgcgaccaccgcttctcgcccgccctcctgggaacgcgctgccctcctgctccgcaccttcaggccgagcaaacctgcacagctgcgccctcgcctgacccaccgcgcccccaaggtccggccgcgcgccgagtccactcaccttccagcccgccgagctgttgctgtcacccttatccttgaagtagggcacgctcttgaccatccactcgtagatctgcgacagcgtgagccgcttctccgccgagctctcgatggccttggtgatgaggtcggcgtaggacaggttgccccacgcgttgcggcgggacgagctgctcttgcgcggctgccccgcgagcggcccagcggcggcggggggcaccggcgggtgctgcgacagcggcccgggcggcgggggctgcggtggcgctgggtgcaggcagcccgcctccgggccctggaagtccccgcacagccccccggtggcggccgcggcggccgccgccgccaccgccgccgccacggagccgggcgcctgcgggaagtcctcgctctcctccagcaagctcaggttgctcatgaagtcggcgctgacagcggcagccgaggccgagggcaggcccgccgcggcgtcggggttggcagccgcgctgcccgacggcgccgggctggaggtggccgagttggactggctaaactccggcctgggcagcggccaggtgcacgagcgcggccggggcagcggctcgaagtccgggtccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagctacttgtacagctcgtccatgccgccggtggagtggcggccctcggcgcgttcgtactgttccacgatggtgtagtcctcgttgtgggaggtgatgtccaacttgatgccgacgatgtaggcgccgggcagctgcacgggcttcttggccttgtaggtggtcttgacctcggaggtgtagtggccgccgtccttcagcttcagcctcatcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctggagggaggagtcctgggtcacggtcaccacgccgccgtcctcgaagttcatcacgcgctcccacttgaagccctcggggaaggacagcttgaagtagtcggggatgtcggcggggtgcttcacgtaggccttggagccgtaggtgaactgaggggacaggatgtcccaggcgaagggcagggggccacccttggtcaccttcagcttagcggtctgaaagccctcgtaggggcggccctcgccctcgccctcgatctcgaactcgtggccgttcacggagccctccatgcgcaccttgaagcgcatgaactccttgatgatggccatgttattctcctcgcccttgctcaccatcgatctccaccacctgaggcgcctcggccatggtgacccccgcccctcccccagccgcaggagagccaagagggggagaacgcagcactgggggcggacggggagggggcgcgaagggacggtccgagatttgggggaacgaagccggtgcggcgagcggacggaaactgggaggaaggcgcggcggagtggaagcgcgagcccagaacttaacttcgcggggccatccacatcgaggctcctcggggtccgccgcacggactggacggccggccagagccgccgggccggggcagagcctgcgccgcgctccagctgacagggccgcggacggaaggacggacggacgccgcgggccgcttgctctccccagcggcgcgcccgctgcgctgctgcctgttgaatgtggcggctgcggcagcggctgctgcgactaccaggccgcccgacttacgggatctgccgccgccccccgcccgcggcggcgcgcgcgccggcccgcccctgaccgacagcccgcgcggccaatgggcatgcggcaccgccgcccgggcagccagtgggcgccgggctgggtggggcccggttttccacggggaggcggcggtgggctggtggggggtagtggggtgtttttctctttcacacactcacctcctttttttttttttggatctctattattttctggtaattctcgagtgtttctgtgattctctcgccttctcagtgttttgattgctaggaagcaaaccagcgtggaggcgccggcgacactttgtttactacggagcagcagagccgagtactcgggaagcccgggtgggaggaggcgctcgctgctccctgacctccgctgcgggccgagcccggcgggctggcagggcagggggccgagggccgggggcgcggggtgggcgggcggaggcggccgcgaggaattctactcaatcgctccctcctggctccacccacgatgtctttgctgaacgacgtggggaagtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 9 - DNA, 2710 bp, Artificial Sequence
  Synthetic pUC57 Backbone Sequence for the Targeting Vectors
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 19 bp, Artificial Sequence
  Synthetic Ngn3 gRNA #4
tggacagtgggcgcacccg

SEQ ID NO: 11 - DNA, 19 bp, Artificial Sequence
  Synthetic Ngn3 gRNA #8
ggacagtgggcgcacccga

SEQ ID NO: 12 - DNA, 19 bp, Artificial Sequence
  Synthetic Foxo1 gRNA #1
caggtggtggagatcgacc

SEQ ID NO: 13 - DNA, 19 bp, Artificial Sequence
  Synthetic Foxo1 gRNA #10
acctgaggcgcctcggcca

SEQ ID NO: 14 - DNA, 19 bp, Artificial Sequence
  Synthetic Tph2 gRNA #1
ctgcagcagggggtactag

SEQ ID NO: 15 - DNA, 19 bp, Artificial Sequence
  Synthetic Tph2 gRNA #5
ttgctggctgcatggatcc

SEQ ID NO: 16 - DNA, 23 bp, Artificial Sequence
  Synthetic Guide #1
ggggcaggaggcgcatccacagg

SEQ ID NO: 17 - DNA, 23 bp, Artificial Sequence
  Synthetic Guide #2
gggcaggaggcgcatccacaggg

SEQ ID NO: 18 - DNA, 21 bp, Artificial Sequence
  Synthetic primer
gacctcatcaccaaggccatc

SEQ ID NO: 19 - DNA, 28 bp, Artificial Sequence
  Synthetic primer
ggcccatcattacattttggcccaggac

SEQ ID NO: 20 - DNA, 21 bp, Artificial Sequence
  Synthetic primer
tttactgttctagtccatgga

SEQ ID NO: 21 - DNA, 21 bp, Artificial Sequence
  Synthetic primer
tccatggactagaacagtaaa

SEQ ID NO: 22 - protein, 110 aa, Homo sapiens
  misc_feature (1)..(110): Human insulin
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 23 - protein, 655 aa, Homo sapiens
  misc_feature (1)..(655): Human FOX01 Protein
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 24 - protein, 444 aa, Homo sapiens
  misc_feature (1)..(444): Human TPH1 Protein
MIEDNKENKDHSLERGRASLIFSLKNEVGGLIKALKIFQEKHVNLLHIESRKSKRRNSEFEIFVDCDINREQLNDIFHLLKSHTNVLSVNLPDNFTLKEDGMETVPWFPKKISDLDHCANRVLMYGSELDADHPGFKDNVYRKRRKYFADLAMNYKHGDPIPKVEFTEEEIKTWGTVFQELNKLYPTHACREYLKNLPLLSKYCGYREDNIPQLEDVSNFLKERTGFSIRPVAGYLSPRDFLSGLAFRVFHCTQYVRHSSDPFYTPEPDTCHELLGHVPLLAEPSFAQFSQEIGLASLGASEEAVQKLATCYFFTVEFGLCKQDGQLRVFGAGLLSSISELKHALSGHAKVKPFDPKITCKQECLITTFQDVYFVSESFEDAKEKMREFTKTIKRPFGVKYNPYTRSIQILKDTKSITSAMNELQHDLDVVSDALAKVSRKPSI

SEQ ID NO: 25 - protein, 490 aa, Homo sapiens
  misc_feature (1)..(490): Human TPH2 Protein
MQPAMMMFSSKYWARRGFSLDSAVPEEHQLLGSSTLNKPNSGKNDDKGNKGSSKREAATESGKTAVVFSLKNEVGGLVKALRLFQEKRVNMVHIESRKSRRRSSEVEIFVDCECGKTEFNELIQLLKFQTTIVTLNPPENIWTEEEELEDVPWFPRKISELDKCSHRVLMYGSELDADHPGFKDNVYRQRRKYFVDVAMGYKYGQPIPRVEYTEEETKTWGVVFRELSKLYPTHACREYLKNFPLLTKYCGYREDNVPQLEDVSMFLKERSGFTVRPVAGYLSPRDFLAGLAYRVFHCTQYIRHGSDPLYTPEPDTCHELLGHVPLLADPKFAQFSQEIGLASLGASDEDVQKLATCYFFTIEFGLCKQEGQLRAYGAGLLSSIGELKHALSDKACVKAFDPKTTCLQECLITTFQEAYFVSESFEEAKEKMRDFAKSITRPFSVYFNPYTQSIEILKDTRSIENVVQDLRSDLNTVCDALNKMNQYLGI

SEQ ID NO: 26 - protein, 490 aa, Homo sapiens
  misc_feature (1)..(490): Human neurogenin 3 protein
MGPVMPPSKKPESSGISVSSGLSQCYGGSGFSKALQEDDDLDFSLPDIRLEEGAMEDEELTNLNWLHESKNLLKSFGESVLRSVSPVQDLDDDTPPSPAHSDMPYDARQNPNCKPPYSFSCLIFMAIEDSPTKRLPVKDIYNWILEHFPYFANAPTGWKNSVRHNLSLNKCFKKVDKERSQSIGKGSLWCIDPEYRQNLIQALKKTPYHPHPHVFNTPPTCPQAYQSTSGPPIWPGSTFFKRNGALLQDPDIDAASAMMLLNTPPEIQAGFPPGVIQNGARVLSRGLFPGVRPLPITPIGVTAAMRNGITSCRMRTESEPSCGSPVVSGDPKEDHNYSSAKSSNARSTSPTSDSISSSSSSADDHYEFATKGSQEGSEGSEGSFRSHESPSDTEEDDRKHSQKEPKDSLGDSGYASQHKKRQHFAKARKVPSDTLPLKKRRTEKPPESDDEEMKEAAGSLLHLAGIRSCLNNITNRTAKGQKEQKETTKN

SEQ ID NO: 27 - DNA, 70 bp, Artificial Sequence
  Synthetic
ggatccatgcagccagcaatgatgatgttttccagtaaatactgggcacggagagggttttccctggatt

SEQ ID NO: 28 - DNA, 23 bp, Artificial Sequence
  Synthetic
gtaaatactgggcacggagaggg

SEQ ID NO: 29 - DNA, 475 bp, Artificial Sequence
  Synthetic
  misc_feature (1)..(1): n is a, c, g, or t
  misc_feature (15)..(15): n is a, c, g, or t
ncgcattgctcttcngcaccagggttctggacagcgccccaagcaggcagctgatcgcacgccccttcctctcaatctccgccagcgctgctactgcccctctagtaccccctgctgcagagaaagaatattacaccgggatccatgcagccagcaatgatgatgttttccagtaaatactgggcacggatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccagg

SEQ ID NO: 30 - DNA, 500 bp, Artificial Sequence
  Synthetic
taaaggagcccggggatgggaggattcgcattgctcttcagcaccagggttctggacagcgccccaagcaggcagctgatcgcacgccccttcctctcaatctccgccagcgctgctactgcccctctagtaccccctgctgcagagaaagaatattacaccgggatccatgcagccagcaatgatgatgttttccagtaaatactgggcacggatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccagg